(19) United States
(12) Patent Application Publication
(10) Pub. No.: US 2023/0125741 A1
(43) Pub. Date: Apr. 27, 2023

(54) MEDIA AND METHODS FOR PRODUCING MESENCHYMAL STEM CELLS

(71) Applicant: Accellta Ltd., Haifa (IL)

(72) Inventors: Michal AMIT, Yuvalim (IL); Hagit DOMEV COHEN, Kiryat-Motzkin (IL)

(73) Assignee: Accellta Ltd., Haifa (IL)

(21) Appl. No.: 18/087,858

(22) Filed: Dec. 23, 2022

Related U.S. Application Data
(63) Continuation of application No. PCT/IL2021/050769, filed on Jun. 23, 2021.
(60) Provisional application No. 63/042,634, filed on Jun. 23, 2020.

(51) Int. Cl. C12N 5/0775

(52) U.S. Cl. CPC C12N 5/0662; C12N 2500/90; C12N 2500/25; C12N 2500/38; C12N 2501/115; C12N 2501/119; C12N 2501/135; C12N 2501/15; C12N 2501/11; C12N 2501/415; C12N 2506/45

(57) ABSTRACT

Culture media capable of promoting differentiation of pluripotent stem cells (PSCs) into mesenchymal stem cells (MSCs) or supporting expansion of MSCs is provided. Methods of differentiation of PSCs into MSCs are provided. Methods of expanding MSCs without differentiation are also provided.

### RELATED APPLICATIONS

This application is a Continuation of PCT Patent Application No. PCT/IL2021/050769 having International filing date of Jun. 23, 2021, which claims the benefit of priority under 35 USC § 119(e) of U.S. Provisional Patent Application No. 63/042,634 filed on Jun. 23, 2020. The contents of the above applications are all incorporated by reference as if fully set forth herein in their entirety.

### FIELD AND BACKGROUND OF THE INVENTION

The present invention, in some embodiments thereof, relates to culture media for culturing pluripotent stem cells, and more particularly, but not exclusively, to novel culture media which can be used to differentiate pluripotent stem cells into mesenchymal stem cells (MSCs), and to novel culture media which can maintain MSCs in a proliferative, multipotent and undifferentiated state.

Mesenchymal stem cells (MSCs) are stem cells having multipotency and self-replication potency. MSCs can differentiate into a variety of cells including cells belonging to the mesenchyme, such as osteoblasts, chondrocytes, adipocytes, and myocytes, as well as into neurocytes and hepatocytes. Furthermore, MSCs are known to have a paracrine effect and a cellular adhesive interaction by self-produced humoral factors. On the basis of these effects, MSCs exert a capability of repairing and regenerating target tissues and cells as well as a capability of controlling an immune response, for example, in anti-inflammation, thereby providing a therapeutic effect on various diseases.

MSCs can be derived from various adult or fetal tissues (e.g. from bone marrow, embryonic yolk sac, placenta, umbilical cord tissues, umbilical cord blood, amniotic fluid, and adipose tissue). Alternatively, unlimited and reproducible fetal or adult MSCs can be produced from pluripotent stem cells (PSCs), either—embryonic stem cells (ESCs) or induced stem cells (iPS cells), respectively.

Human embryonic stem cells (hESCs) can be used as a reliable source for generating high-quality human MSCs. hESCs are proliferative, undifferentiated stem cells capable of differentiating into cells of all three embryonic germ layers. Furthermore, hESC can be propagated and expanded in vitro indefinitely, providing a potentially inexhaustible and donorless source of cells for human therapy.

Human iPS cells are induced pluripotent stem cells obtained by introducing genes (e.g. Oct4, Sox2, Klf4 and c-Myc, or Oct4, Sox2, Nanog and Lin28) into somatic cells [Takahashi K. Cell (2007) 131: 861-872; and Yu J. et al., Science (2007) 318: 1917-1920]. iPS cells have similar properties to hESC and can differentiate into representative tissues of the three embryonic germ layers both in vitro and in vivo, under specific induction conditions. Improvements of iPS cells derivation methods include the use of plasmids instead of viral vectors or derivation without any integration to the genome, which may simplify the use of iPS cells for clinical applications [Yu J, et al., Science (2009) 324: 797-801].

There are various methods to differentiate human ESCs and iPS cells into MSCs.

U.S. Pat. No. 10,351,825 provides methods of producing MSCs from iPS cells in which the iPS cells are cultured in the presence of a TGF-beta inhibitor (e.g. SB431542) in an atmosphere containing 7-8 vol. % CO₂ for 20-35 days. The cells are then transferred to a culture dish having a hydrophilic surface, and the cells are cultured in a medium containing a TGF-beta inhibitor for a period of time sufficient to produce MSCs.

U.S. Patent Application No. 20170290864 provides methods of differentiating ESCs or iPS cells into MSCs under conditions in which the ESCs or iPS cells develop through an intermediate differentiation of trophoblasts. Specifically, pluripotent stem cells are cultured in a medium comprising a bone morphogenetic protein-4 (BMP-4), and optionally a TGF-beta inhibitor (e.g. SB431542, A83-01 or ALK5 inhibitor), and the trophoblasts are further differentiated into MSC derived cells by culturing the trophoblasts in gelatin-, vitronectin-, laminin-, fibronectin-, Matrigel- or collagen-coated plates, in a MSC growth medium containing LIF, bFGF, PDGF, or a combination thereof.

PCT publication no. WO/2011/091475 provides methods for generating MSCs from pluripotent cells, the method comprising (i) differentiating ESC or iPS cells attached to a surface of a culture vessel in the presence of an inhibitor of endogenous activin and TGFβ signaling (e.g. SB431542) and (ii) passaging the resultant cells in the presence of a MSC medium so as to produce MSCs.

Additional background art includes U.S. Pat. No. 10,214,722 and Schubert and Smitz: “In vitro culture of human primordial follicles”, In “Fertility Cryopresservation”, ed. Ri-Cheng Chian and Patrick Quinn. Published by Cambridge University Press. Co University Press (2020) pages 200-212.

### SUMMARY OF THE INVENTION

According to an aspect of some embodiments of the present invention there is provided a defined culture medium comprising a basal medium and an effective amount of a knockout serum replacement (KoSR) and ITS (Insulin, Transferrin and Selenium), wherein the culture medium is serum-free, and further wherein the culture medium is capable of promoting differentiation of pluripotent stem cells (PSCs) into mesenchymal stem cells (MSCs) or supporting expansion of MSCs.

According to an aspect of some embodiments of the present invention there is provided a culture medium comprising a basal medium comprising DMEM/F12 and MEM alpha at a volume ratio ranging between 0.4 to 2.3 DMEM/F12 to MEM alpha, wherein the culture medium is capable of promoting differentiation of pluripotent stem cells (PSCs) into mesenchymal stem cells (MSCs) or supporting expansion of MSCs.

According to an aspect of some embodiments of the present invention there is provided a defined culture medium comprising a basal medium comprising DMEM/F12, knockout serum replacement (KoSR) at a concentration of at least 5%, and ITS (Insulin, Transferrin and Selenium) at a concentration of at least 0.5%, wherein the culture medium is serum-free, and further wherein the culture medium is capable of promoting differentiation of pluripotent stem cells (PSCs) into mesenchymal stem cells (MSCs) or supporting expansion of MSCs.

According to an aspect of some embodiments of the present invention there is provided a defined culture medium comprising a basal medium comprising DMEM/F12, knockout serum replacement (KoSR) at a concentration of at least 5%, ITS (Insulin, Transferrin and Selenium) at a concentration of at least 0.5% and L-ascorbic acid at a concentration of at least 25 μg/ml, wherein the culture medium is serum-free, and further wherein the culture medium is capable of promoting differentiation of pluripotent stem cells (PSCs) into mesenchymal stem cells (MSCs) or supporting expansion of MSCs.

According to an aspect of some embodiments of the present invention there is provided a defined culture medium comprising a basal medium comprising DMEM HG, knockout serum replacement (KoSR) at a concentration of at least 5%, ITS (Insulin, Transferrin and Selenium) at a concentration of at least 0.5%, L-ascorbic acid at a concentration of at least 25 μg/ml and Sodium pyruvate at a concentration of at least 25 μg/ml, wherein the culture medium is serum-free, and further wherein the culture medium is capable of promoting differentiation of pluripotent stem cells (PSCs) into mesenchymal stem cells (MSCs) or supporting expansion of MSCs.

According to an aspect of some embodiments of the present invention there is provided a culture medium comprising a basal medium comprising DMEM/F12 and MEM alpha at a volume ratio ranging between 0.6 to 1.5 DMEM/F12 to MEM alpha, a knockout serum replacement (KoSR) at a concentration of at least 3%, ITS (Insulin, Transferrin and Selenium) at a concentration of at least 0.1%, and serum at a concentration of at least 5%, and further wherein the culture medium is capable of promoting differentiation of pluripotent stem cells (PSCs) into mesenchymal stem cells (MSCs) or supporting expansion of MSCs.

According to an aspect of some embodiments of the present invention there is provided a method of expanding mesenchymal stem cells (MSCs) without differentiation, the method comprising culturing the MSCs in the culture medium of some embodiments of the invention, thereby culturing the MSCs under culturing conditions which allow expansion of the MSCs without differentiation.

According to an aspect of some embodiments of the present invention there is provided a method of generating mesenchymal stem cells (MSCs) from pluripotent stem cells (PSCs), the method comprising culturing the PSCs in a suspension culture in the culture medium of some embodiments of the invention under culturing conditions suitable for differentiation of the PSCs to MSCs, thereby generating the MSCs.

According to an aspect of some embodiments of the present invention there is provided a method of generating mesenchymal stem cells (MSCs) from pluripotent stem cells (PSCs), the method comprising culturing the PSCs in a suspension culture in a culture medium comprising a basal medium comprising MEM alpha, serum and ITS (Insulin, Transferrin and Selenium), under culturing conditions suitable for differentiation of the PSCs to MSCs, thereby generating the MSCs.

According to an aspect of some embodiments of the present invention there is provided a method of generating mesenchymal stem cells (MSCs) from pluripotent stem cells (PSCs), the method comprising culturing the PSCs in a suspension culture in a culture medium comprising a basal medium comprising MEM alpha, serum at a concentration of at least 5%, and ITS (Insulin, Transferrin and Selenium) at a concentration of at least 1%, under culturing conditions suitable for differentiation of the PSCs to the MSCs, thereby generating the MSCs.

According to an aspect of some embodiments of the present invention there is provided an isolated population of mesenchymal stem cells (MSCs) in a suspension culture generated according to the method of some embodiments of the invention.

According to an aspect of some embodiments of the present invention there is provided a cell culture comprising MSCs and the culture medium of some embodiments of the invention.

According to an aspect of some embodiments of the present invention there is provided a cell culture comprising PSCs and the culture medium of some embodiments of the invention.

According to an aspect of some embodiments of the present invention there is provided a cell culture comprising PSCs and a culture medium comprising a basal medium comprising MEM alpha, serum and ITS (Insulin, Transferrin and Selenium).

According to an aspect of some embodiments of the present invention there is provided a cell culture comprising PSCs and a culture medium comprising a basal medium comprising MEM alpha, serum at a concentration of at least 5%, and ITS (Insulin, Transferrin and Selenium) at a concentration of at least 1%.

According to some embodiments of the invention, the basal medium comprises DMEM/F12.

According to some embodiments of the invention, the basal medium comprises DMEM high glucose (DMEM HG).

According to some embodiments of the invention, the volume ratio of DMEM/F12 to MEM alpha ranges between 0.6 to 1.5 DMEM/F12 to MEM alpha.

According to some embodiments of the invention, the culture medium further comprises at least one of knockout serum replacement (KoSR) and ITS (Insulin, Transferrin and Selenium).

According to some embodiments of the invention, the concentration of KoSR is at least 2.5%.

According to some embodiments of the invention, the concentration of KoSR is at most 15%.

According to some embodiments of the invention, the concentration of KoSR is 2.5-15%.

According to some embodiments of the invention, the concentration of KoSR is 5-15%.

According to some embodiments of the invention, the concentration of ITS is at least 0.1%.

According to some embodiments of the invention, the concentration of ITS is at most 3%.

According to some embodiments of the invention, the concentration of ITS is 0.1-3%.

According to some embodiments of the invention, the concentration of ITS is 0.5-3%.

According to some embodiments of the invention, the culture medium further comprises at least one of glucose, L-ascorbic acid and Sodium pyruvate.

According to some embodiments of the invention, the concentration of the glucose is at least 1 gr/Liter.

According to some embodiments of the invention, the concentration of the glucose is at most 5 gr/Liter.

According to some embodiments of the invention, the concentration of the glucose is 1 gr/Liter-5 gr/Liter.

According to some embodiments of the invention, the concentration of the L-ascorbic acid is at least 20 μg/ml.

According to some embodiments of the invention, the concentration of the L-ascorbic acid is at most 200 μg/ml.

According to some embodiments of the invention, the concentration of the L-ascorbic acid is 20-200 μg/ml.

According to some embodiments of the invention, the concentration of the Sodium pyruvate is at least 20 μg/ml.

According to some embodiments of the invention, the concentration of the Sodium pyruvate is at most 200 μg/ml.

According to some embodiments of the invention, the concentration of the Sodium pyruvate acid is 20-200 μg/ml.

According to some embodiments of the invention, the culture medium further comprises serum.

According to some embodiments of the invention, the concentration of serum is at least 3%.

According to some embodiments of the invention, the concentration of serum is at most 30%.

According to some embodiments of the invention, the concentration of serum is 3-30%.

According to some embodiments of the invention, the concentration of serum is 5-30%.

According to some embodiments of the invention, the culture medium further comprises basic fibroblast growth factor (bFGF).

According to some embodiments of the invention, the culture medium further comprises basic fibroblast growth factor (bFGF) at a concentration of at least 1 ng/ml.

According to some embodiments of the invention, the bFGF comprises FGF2 or FGF4.

According to some embodiments of the invention, the bFGF comprises FGF2 and FGF4.

According to some embodiments of the invention, the FGF2 is at a concentration of at least 1 ng/ml.

According to some embodiments of the invention, the FGF2 is at a concentration of at most 100 ng/ml.

According to some embodiments of the invention, the FGF2 is at a concentration of 1-100 ng/ml.

According to some embodiments of the invention, the FGF4 is at a concentration of at least 1 ng/ml.

According to some embodiments of the invention, the FGF4 is at a concentration of at most 100 ng/ml.

According to some embodiments of the invention, the FGF4 is at a concentration of 1-100 ng/ml.

According to some embodiments of the invention, the culture medium further comprises at least one of platelet-derived growth factor (PDGF), transforming growth factor beta (TGFβ), epidermal growth factor (EGF) and WNT-3a.

According to some embodiments of the invention, the PDGF comprises PDGF-BB.

According to some embodiments of the invention, the PDGF is at a concentration of at least 5 ng/ml.

According to some embodiments of the invention, the PDGF is at a concentration of at most 100 ng/ml.

According to some embodiments of the invention, the PDGF is at a concentration of 5-100 ng/ml.

According to some embodiments of the invention, the TGFβ comprises TGFβ1 or TGFβ3.

According to some embodiments of the invention, the TGFβ is at a concentration of at least 5 ng/ml.

According to some embodiments of the invention, the TGFβ is at a concentration of at most 50 ng/ml.

According to some embodiments of the invention, the TGFβ is at a concentration of 5-50 ng/ml.

According to some embodiments of the invention, the EGF is at a concentration of at least 1 ng/ml.

According to some embodiments of the invention, the EGF is at a concentration of at most 100 ng/ml.

According to some embodiments of the invention, the EGF is at a concentration of 1-100 ng/ml.

According to some embodiments of the invention, the WNT-3a is at a concentration of at least 10 ng/ml.

According to some embodiments of the invention, the WNT-3a is at a concentration of at most 200 ng/ml.

According to some embodiments of the invention, the WNT-3a is at a concentration of 10-200 ng/ml.

According to some embodiments of the invention, the culturing comprise a 2D culture.

According to some embodiments of the invention, the culturing comprise a suspension (3D) culture.

According to some embodiments of the invention, the culturing is for at least 5 passages.

According to some embodiments of the invention, the culturing is for up to 25 passages.

According to some embodiments of the invention, the MSCs are capable of differentiating into any one of an adipogenic lineage, an osteoblastic lineage, and a chondrogenic lineage.

According to some embodiments of the invention, the culturing is affected under culturing conditions devoid of substrate adherence.

According to some embodiments of the invention, the suspension culture of PSCs comprises clumps.

According to some embodiments of the invention, the suspension culture comprising the clumps comprises cell clusters comprising more than about 250 pluripotent stem cells.

According to some embodiments of the invention, the suspension culture of PSCs is devoid of clumps.

According to some embodiments of the invention, the suspension culture devoid of the clumps comprises single cells or small clusters, each of the small clusters comprising no more than about 200 pluripotent stem cells.

According to some embodiments of the invention, the culturing is affected for 3-14 days.

According to some embodiments of the invention, at least 40% of the MSCs are characterized by a CD105⁺/CD146⁺/CD90⁺/CD44⁺/CD31⁻/CD34⁻/CD45⁻ expression signature.

According to some embodiments of the invention, the MSCs comprise human MSCs.

According to some embodiments of the invention, the MSCs comprise adult MSCs.

According to some embodiments of the invention, the MSCs comprise fetal MSCs.

According to some embodiments of the invention, the MSCs comprise PSC-derived MSCs.

According to some embodiments of the invention, the PSCs comprise human PSCs.

According to some embodiments of the invention, the PSCs comprise embryonic stem cells (ESCs).

According to some embodiments of the invention, the PSCs comprise induced pluripotent stem cells (iPSs).

Unless otherwise defined, all technical and/or scientific terms used herein have the same meaning as commonly understood by one of ordinary skill in the art to which the invention pertains.

Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-D depict the morphology of hESCs during mesenchymal differentiation. FIGS. 1A-B illustrates cell aggregates; FIGS. 1C-D illustrates single cells; FIGS. 1A and 1C illustrate the cell morphology of aggregates and single cells following 7 days of differentiation in suspension culture; FIGS. 1B and 1D illustrate the cell morphology following re-plating of the cells back to adherent culture. Scale bar: 100 μm.

FIGS. 2A-E depict representative FACS analyses of differentiation of hESCs (I3) towards MSCs. hESCs were cultured for 7 days in suspension as aggregates or as single cells. Blue histogram—indicates specific antibody, red histogram—illustrates matched isotype control. FIG. 2A illustrates the results for CD146 and CD73 expression; FIG. 2B illustrates the results for CD105 expression; FIG. 2C illustrates the results for CD90 expression; FIG. 2D illustrates the results for CD45 expression; and FIG. 2E illustrates the results for and CD31 expression. Of note, more than 95% of the cells were positive for the expression of CD146, CD73, CD105 and CD90 (FIGS. 2A-C) and were negative for the expression of CD31 and CD45 (FIGS. 2D-E).

FIGS. 3A-C depict in-vitro differentiation of hESC-MSCs into mesodermal derivatives. Following mesenchymal differentiation in 3D suspension cultures, the cells were re-plated in 2D adherent cultures and were subjected to adipogenic, osteogenic and chondrogenic differentiation. Multilocular adipocytes stained positive for Oil red (FIG. 3A), osteoblasts stained positive to Alizarin red (FIG. 3B), and chondroblasts stained positive to Alcian blue (FIG. 3C).

FIG. 4 depicts the cumulative cell counts of MSCs expanded in MSC basal medium (MSC-Diff 0-F12 medium) comprising different concentrations of bFGF.

FIG. 5 depicts representative morphology of MSCs expanded in MSC basal medium (MSC-Diff 0-F12 medium) comprising different concentrations of bFGF.

FIGS. 6A-C depict cell counts in umbilical cord MSCs (MSC-UCs) expanded in MSC basal medium (MSC-Diff 0-F12 medium) comprising different concentrations and combinations of PDGF-BB, EGF and bFGF (throughout the culture period). FIG. 6A illustrates the fold increase per day; FIG. 6B illustrates the population doubling time (PDT); and FIG. 6C illustrates the cumulative results of cell count. Cells were counted over the course of approximately 60 days. Parallel lines represent the days the cells were counted on. (*)P<0.5, (**)P<0.01, (***)P<0.001.

FIG. 7 depicts representative morphology of MSC-UCs expanded in MSC basal medium (MSC-Diff 0-F12 medium) comprising different concentrations and combinations of PDGF-BB, EGF and bFGF (throughout the culture period).

FIG. 8 depicts relative proliferation of adult human umbilical cord MSCs (ahMSC-UCs) expanded in MSC basal medium (MSC-Diff 0-F12 medium) comprising different concentrations and combinations of bFGF, PDGF-BB, TGFβ1 and WNT-3a (throughout the culture period). Sample results of ahUC-MSC after 3 passages in DMEM/F12 basal medium supplemented with different concentrations of growth factors and cytokines. Control supplemented only with 50 ng/ml bFGF.

FIGS. 9A-G depicts representative morphology of ahMSC-UCs expanded in MSC basal medium (MSC-Diff 0-F12 medium) comprising different concentrations and combinations of bFGF, PDGF-BB, TGFβ1 and WNT-3a (throughout the culture period). Representative light microscopy images (×10 magnification) of ahMSC-UC grown in example results of DMEM/F12 basal medium.

FIG. 10 depicts relative proliferation of adult human umbilical cord MSCs (ahMSC-UCs) expanded in high glucose DMEM media (MSC-Diff 0-HG) comprising different concentrations and combinations of bFGF, FGF4, TGFβ3 and WNT-3a (throughout the culture period). Sample results of ahUC-MSC after 3 passages in DMEM HG basal medium supplemented with different concentrations of growth factors and cytokines. Control supplemented only with 50 ng/ml bFGF.

FIGS. 11A-J depict representative morphology of ahMSC-UCs expanded in high glucose DMEM media (MSC-Diff 0-HG) comprising different concentrations and combinations of bFGF, FGF4, TGFβ3 and WNT-3a (throughout the culture period). Representative light microscopy images (×10 magnification) of ahMSC-UC grown in example results of DMEM HG basal.

FIGS. 12A-E depict ahMSC-UC cell density in MSC-Diff 51. Representative light microscopy images (×10 magnification) of ahMSC-UC in different passages (passages 1 to 11) grown in MSC-Diff 51 medium. Of note, ahMSC-UC maintained their morphology following long-term culture in the indicated media.

FIG. 12F depicts the cumulative cell number of hUC-MSCs grown in MSC-Diff 51 medium. ahMSC-UC cumulative cell number following 72 days (10 passages) of growth in MSC-Diff 51 medium. Of note, hUC-MSCs exhibited significant proliferation during long term culture in the indicated media.

FIG. 13 depicts FACS analysis of human induced pluripotent stem cells (hiPSCs) in 3 different MSC-differentiation media. iPSCs. HiPSCs (Dyr0100) were cultured with the indicated media, the cells were stained for CD90 at days 7 or 8, and 14 following differentiation. Red histogram represents cells stained for the indicated marker; blue histogram represents unstained cells. Of note, MSC markers first emerge following 7-8 days of differentiation and widespread expression of the mesenchymal marker CD90 was observed through differentiation in all tested differentiation media.

FIG. 14 depicts percentage change in markers for iPSC-derived MSCs after 8 days in differentiation media (sample results). Percentage change in markers for iPSC-derived MSCs after 8 days in MSC-Diff 36, 37 and 38 compared to day 0. Of note, the expression of the mesenchymal markers: CD73, CD105 and CD146 increased, while the expression of CD45 (hematopoietic marker) decreased.

FIG. 15 depicts OCT4 expression levels in iPSC-derived MSCs cells after 2 weeks in the indicated differentiation media. iPSCs were cultured in 3 different MSC-Diff media and stained for the key pluripotency marker OCT4. OCT4 expression level in cells cultured in MSC-Diff 36, 37 and 38 decreased significantly as mesenchymal differentiation progressed.

FIGS. 16A-H depict the osteogenic potential of iPSC-derived MSCs (induced after 7 and 14 days in mesenchymal differentiation media) as manifested by Alizarin red S staining for calcium deposits. Representative light microscopy images (×10 magnification) of Alizarin red S staining indicating osteogenic potential. iPSC-derived MSCs (resulted from 7 or 14 days of mesenchymal differentiation in 3D culture) were further differentiated into osteocytes for 30 days or 16 days respectively. Of note, the cells positively stained with Alizarin red, indicating osteogenesis. FIGS. 16A and 16E—human Umbilical cord-derived MSCs (served as positive control). FIGS. 16B and 16F—iPSC-derived MSCs (from MSC-Diff 36 induction) in 3D culture. FIGS. 16C and 16G—iPSC-derived MSCs (from MSC-Diff 37 induction) in 3D culture. FIGS. 16D and 16H—iPSC-derived MSCs (from MSC-Diff 38 induction) in 3D culture.

FIGS. 17A-D depict the adipogenic potential of iPSC-derived MSCs as manifested by Oil Red O staining. Representative light microscopy images (×20 magnification) of Oil red O staining indicating adipogenic potential. Of note, iPSC-derived MSCs (generated from mesenchymal induction in 3D culture), were positively stained with Oil Red, indicating adipogenesis. FIG. 17A—human Umbilical cord-derived MSCs (served as positive control). FIG. 17B—iPSC-derived MSCs (from MSC-Diff 36 induction) in 3D culture. FIG. 17C—iPSC-derived MSCs (from MSC-Diff 37 induction) in 3D culture. FIG. 17D—iPSC-derived MSCs (from MSC-Diff 38 induction) in 3D culture.

FIGS. 18A-B depict FACS analyses of hiPSC-derived MSCs for MSC markers in 2 different MSC-differentiation media following 2D cell adherence assay (sample results). hiPSC-derived MSCs (induced towards mesenchymal differentiation in 3D culture) were tested for plastic adherence by seeding the cells in plastic tissue culture plates. The cells were further cultured in the indicated media for 15 days and analyzed for the expression of MSC markers: CD73, CD105, CD146 (FIG. 18A), and CD90 (FIG. 18B). Red histogram represents cells stained for the indicated marker; blue histogram represents unstained cells. Of note, the hiPSC-derived MSCs (from 3D culture) maintained high expression of the mesenchymal markers in 2D culture as well.

FIG. 19 depicts the percentage change in markers of iPSC-derived MSCs (from 3D culture) following 15 days in 2D cell adherence assay (sample results). hiPSC-derived MSCs (induced towards mesenchymal differentiation in 3D culture) were tested for plastic adherence by seeding the cells in plastic tissue culture plates. The cells were further cultured in the indicated media for 15 days and analyzed for the percentage change in the MSC markers: CD73, CD105, CD146, in addition to the hematopoietic marker CD45.

FIGS. 20A-C depict Alizarin red S staining for calcium deposits in osteocytes differentiated from iPSC-derived MSCs following adherence assay (sample results). Representative light microscopy images (×10 magnification) of positive alizarin red S staining indicating osteogenic potential of hiPSC-derived MSCs following adherence assay. hiPSC-derived MSCs (generated in 3D culture in the indicated media) were seeded in plastic tissue culture plates and further cultured with osteogenic media for 16 days. FIG. 20A—ahUC-MSCs (Positive control). FIG. 20B—iPSC-derived MSCs (from MSC-Diff 37 induction) in 3D culture. FIG. 20C—iPSC-derived MSCs (from MSC-Diff 38 induction) in 3D culture.

FIGS. 21A-C depict Oil red O staining for lipid droplets in adipocytes differentiated from iPSC-derived MSCs following adherence assay (sample results). Representative light microscopy images (×20 magnification) of Oil red O staining indicating adipogenic potential of hiPSC-derived MSCs following adherence assay. hiPSC-derived MSCs (generated in 3D culture in the indicated media) were seeded in plastic tissue culture plates and further cultured with osteogenic media for 23 days. FIG. 21A—ahUC-MSCs (positive control). FIG. 21B—iPSC-derived MSCs (from MSC-Diff 37 induction) in 3D culture. FIG. 21C—iPSC-derived MSCs (from MSC-Diff 38 induction) in 3D culture.

FIGS. 22A-B depict OCT4 and Nanog expression levels in ESC-derived MSCs cells after 8 days in the indicated media. Human embryonic stem cells were cultured in 3 different MSC-Diff media for 8 days in 3D culture. The cells were analyzed for OCT4 (FIG. 22A) and Nanog (FIG. 22B) expression. Of note, OCT4 and Nanog expression levels decreased significantly during mesenchymal differentiation.

FIGS. 23A-D depict Osteogenic potential of ESC-derived MSCs (induced after 7 days in mesenchymal differentiation media) as manifested by positive Alizarin red S staining for calcium deposits. Representative light microscopy images (×10 magnification) of alizarin red S staining indicating osteogenic potential. ESC-derived MSCs (generated following 7 days of mesenchymal differentiation in 3D culture) were further differentiated into osteocytes for 30 days. FIG. 23A—ahUC-MSCs (positive control). FIG. 23B—ESC-derived MSCs (from MSC-Diff 36 induction) in 3D culture. FIG. 23C—ESC-derived MSCs (from MSC-Diff 37 induction) in 3D culture. FIG. 23D—ESC-derived MSCs (from MSC-Diff 38 induction) in 3D culture.

### DESCRIPTION OF SPECIFIC EMBODIMENTS OF THE INVENTION

The present invention, in some embodiments thereof, relates to culture media for culturing pluripotent stem cells, and more particularly, but not exclusively, to novel culture media which can be used to differentiate pluripotent stem cells into mesenchymal stem cells (MSCs), and to novel culture media which can maintain MSCs in a proliferative, multipotent and undifferentiated state.

The principles and operation of the present invention may be better understood with reference to the drawings and accompanying descriptions.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Mesenchymal stem cells (MSCs) are multipotent cells which can be derived from various adult or fetal tissues and can further differentiate in-vitro into mesodermal derivatives such as: adipocytes, osteocytes, and chondrocytes, and also (with a lesser extent) into ectodermal or endodermal derivatives; neurons or hepatocytes respectively. MSCs are traditionally cultured in 2D adherent culture. Human pluripotent stem cells (hPSCs), either—embryonic or induced, can provide alternative, unlimited and reproducible source of adult or fetal MSCs.

The use of MSCs in therapeutic applications is desirable due to their capacity for self-renewal and multi-lineage differentiation. However, MSCs tend to lose their stem cell properties under conventional cell culture conditions, such as when cultured on tissue culture plastic.

While reducing the present invention to practice, the present inventors have uncovered novel, specific and efficient media for the directed differentiation of hPSCs towards MSCs in non-adherent, carrier-free suspension culture (as cell aggregates or as single cells). Following a few days in differentiation media (e.g. 3-10 days), the presently described protocols generated bona-fide MSCs with high-rate differentiation towards osteoblasts, adipocytes and chondroblasts in-vitro. Furthermore, the present inventors uncovered novel culture media for culturing adult, fetal and PSC-derived MSCs in 2D adherent cultures (with or without coating) or in a suspension (3D) culture. The culture media enabled expansion of MSCs for a prolonged period of time (e.g. for 20-25 passages) while maintaining their undifferentiated state. Altogether, the novel culture media identified herein can be used for an efficient and rapid generation of MSCs from PSCs as well as for prolonged expansion and maintenance of MSCs in an undifferentiated state. Furthermore, the MSCs generated by the described protocols can be used as an unlimited source of proliferative, multipotent, undifferentiated mesenchymal stem cells for various cell based therapies.

Thus, according to one aspect of the present invention there is provided a defined culture medium comprising a basal medium and an effective amount of a knockout serum replacement (KoSR) and ITS (Insulin, Transferrin and Selenium), wherein the culture medium is serum-free, and further wherein the culture medium is capable of promoting differentiation of pluripotent stem cells (PSCs) into mesenchymal stem cells (MSCs) or supporting expansion of MSCs.

According to one aspect of the present invention there is provided a culture medium comprising a basal medium comprising DMEM/F12 and MEM alpha at a volume ratio ranging between 0.4 to 2.3 DMEM/F12 to MEM alpha, wherein the culture medium is capable of promoting differentiation of pluripotent stem cells (PSCs) into mesenchymal stem cells (MSCs) or supporting expansion of MSCs.

As used herein the term “pluripotent stem cells” or “PSC” refers to cells which are capable of differentiating into cells of all three embryonic germ layers (i.e., endoderm, ectoderm and mesoderm). The phrase “pluripotent stem cells” may read on embryonic stem cells (ESCs) and/or induced pluripotent stem cells (iPS cells).

The term “embryonic stem cells” or “ESC” as used herein refers to cells which are obtained from the embryonic tissue formed after gestation (e.g., blastocyst) before implantation (i.e., a preimplantation blastocyst); extended blastocyst cells (EBCs) which are obtained from a post-implantation/pre-gastrulation stage blastocyst (see WO2006/040763]; and/or embryonic germ (EG) cells which are obtained from the genital tissue of a fetus any time during gestation, preferably before 10 weeks of gestation.

According to some embodiments of the invention, the pluripotent stem cells of the invention are embryonic stem cells, such as from a human or primate (e.g., monkey) origin.

The embryonic stem cells of the invention can be obtained using well-known cell-culture methods. For example, human embryonic stem cells can be isolated from human blastocysts. Human blastocysts are typically obtained from human in vivo preimplantation embryos or from in vitro fertilized (IVF) embryos. Alternatively, a single cell human embryo can be expanded to the blastocyst stage. For the isolation of human ES cells the zona pellucida is removed from the blastocyst and the inner cell mass (ICM) is isolated by immunosurgery, in which the trophectoderm cells are lysed and removed from the intact TCM by gentle pipetting. The TCM is then plated in a tissue culture flask containing the appropriate medium which enables its outgrowth. Following 9 to 15 days, the ICM derived outgrowth is dissociated into clumps either by a mechanical dissociation or by an enzymatic degradation and the cells are then re-plated on a fresh tissue culture medium. Colonies demonstrating undifferentiated morphology are individually selected by micropipette, mechanically dissociated into clumps, and re-plated. Resulting ES cells are then routinely split every 4-7 days. For further details on methods of preparation human ES cells see Thomson et al., [U.S. Pat. No. 5,843,780; Science 282: 1145, 1998; Curr. Top. Dev. Biol. 38: 133, 1998; Proc. Natl. Acad. Sci. USA 92: 7844, 1995]; Bongso et al., [Hum Reprod 4: 706, 1989]; and Gardner et al., [Fertil. Steril. 69: 84, 1998].

It will be appreciated that commercially available stem cells can also be used with this aspect of the present invention. Human ES cells can be purchased from the NIH human embryonic stem cells registry (www(dot)//escr(dot)nih(dot)gov). Non-limiting examples of commercially available embryonic stem cell lines are BG01, BG02, BG03, BG04, CY12, CY30, CY92, CY10, TE03, TE04 and TE06.

Extended blastocyst cells (EBCs) can be obtained from a blastocyst of at least nine days post fertilization at a stage prior to gastrulation. Prior to culturing the blastocyst, the zona pellucida is digested [for example by Tyrode's acidic solution (Sigma Aldrich, St Louis, Mo., USA)] so as to expose the inner cell mass. The blastocysts are then cultured as whole embryos for at least nine and no more than fourteen days post fertilization (i.e., prior to the gastrulation event) in vitro using standard embryonic stem cell culturing methods.

Embryonic germ (EG) cells are prepared from the primordial germ cells obtained from fetuses of about 8-11 weeks of gestation (in the case of a human fetus) using laboratory techniques known to anyone skilled in the arts. The genital ridges are dissociated and cut into small chunks which are thereafter disaggregated into cells by mechanical dissociation. The EG cells are then grown in tissue culture flasks with the appropriate medium. The cells are cultured with daily replacement of medium until a cell morphology consistent with EG cells is observed, typically after 7-30 days or 1-4 passages. For additional details on methods of preparation human EG cells see Shamblott et al., [Proc. Natl. Acad. Sci. USA 95: 13726, 1998] and U.S. Pat. No. 6,090,622.

The phrase “induced pluripotent stem (iPS) cell” (or embryonic-like stem cell) as used herein refers to a proliferative and pluripotent stem cell which is obtained by de-differentiation of a somatic cell (e.g., an adult somatic cell).

According to some embodiments of the invention, the iPS cell is characterized by a proliferative capacity which is similar to that of ESCs and thus can be maintained and expanded in culture for an almost unlimited time.

IPS cells can be endowed with pluripotency by genetic manipulation which re-programs the cell to acquire embryonic stem cells characteristics. For example, the iPS cells of the invention can be generated from somatic cells by induction of expression of Oct-4, Sox2, Kfl4 and c-Myc in a somatic cell essentially as described in Takahashi and Yamanaka Cell (2006) 126: 663-676; Takahashi et al., Cell (2007) 131(5):861-72; Meissner et al., Nat Biotechnol (2007) 25(10):1177-81; and Okita et al., Nature (2007) 448: 313-318. Additionally or alternatively, the iPS cells of the invention can be generated from somatic cells by induction of expression of Oct4, Sox2, Nanog and Lin28 essentially as described in Yu et al., Science (2007) 318: 1917-20; and Nakagawa et al., Nat Biotechnol (2008) 26(1):101-6. It should be noted that the genetic manipulation (re-programming) of the somatic cells can be performed using any known method such as using plasmids or viral vectors, or by derivation without any integration to the genome [Yu et al., Science (2009) 324: 797-801].

The iPS cells of the invention can be obtained by inducing de-differentiation of embryonic fibroblasts [Takahashi and Yamanaka (2006), supra; Meissner et al. (2007), supra], fibroblasts formed from hESCs [Park et al, Nature (2008) 10; 451(7175): 141-6], Fetal fibroblasts [Yu et al. (2007), supra; Park et al. (2008), supra], foreskin fibroblast [Yu et al. (2007), supra; Park et al. (2008), supra], adult dermal and skin tissues [Hanna et al., Science. (2007) 318:1920-1923; Lowry et al., Proc Natl Acad Sci USA (2008) 105(8): 2883-8], B-lymphocytes [Hanna et al (2007), supra] and adult liver and stomach cells [Aoi et al., Science (2008) 321(5889):699-702].

IPS cell lines are also available via cell banks such as the WiCell bank. Non-limiting examples of commercially available iPS cell lines include the iPS foreskin clone 1 [WiCell Catalogue No. iPS(foreskin)-1-DL-1], the iPSIMR90 clone 1 [WiCell Catalogue No. iPS(IMR90)-1-DL-1], and the iPSIMR90 clone 4 [WiCell Catalogue No. iPS(IMR90)-4-DL-1].

According to some embodiments of the invention, the induced pluripotent stem cells are human induced pluripotent stem cells.

As used herein the term “mesenchymal stem cells” or “MSC” refers to stem cells derived from any tissue generally classified as mesenchymal. A tissue classified as mesenchymal includes bone marrow, placenta, adipose tissue, and dermal tissue. Examples of mesenchymal stem cells derived from these tissues include, for example, bone marrow-derived mesenchymal stem cells (BM-MSC), placental-derived mesenchymal stem cells, cord blood-derived mesenchymal stem cells (CB-MSC), adipose tissue-derived mesenchymal stem cells, dermal-derived mesenchymal stem cells and dental pulp mesenchymal stem cells.

According to one embodiment, the MSCs are of a fetal origin (e.g. CB-MSC, placental-derived MSC).

According to one embodiment, the MSCs are of an adult origin (i.e. post-fetal, i.e., an organism from the neonate stage through the end of life, and includes, for example, MSCs obtained from bone marrow or adipose tissues).

MSCs are typically capable of giving rise to differentiated cells in multiple mesenchymal lineages, including e.g. osteoblasts, adipocytes, myoblasts, chondroblasts, and fibroblasts. Generally, mesenchymal stem cells also have one or more of the following properties: an ability to undergo asynchronous or asymmetric replication, i.e. where the two daughter cells after division can have different phenotypes; extensive self-renewal capacity; and clonal regeneration of the tissue in which they exist, for example, the non-hematopoietic cells of bone marrow.

MSCs may be characterized by both the presence of certain cell surface markers and the absence of certain cell surface markers, as discussed below. MSCs may also be identified by functional assays both in vitro and in vivo, particularly assays relating to the ability of stem cells to give rise to multiple differentiated progeny, assays for responsiveness to canonical WNT signaling, and the like.

Human MSCs may be characterized by expression of a number of cell-surface proteins, including but not limited to, CD54, CD9, CD29, CD44, CD56, CD61, CD63, CD71, CD73, CD90, CD97, CD98, CD99, CD105, CD106, CD112, CD146, CD155, CD166, CD276, and CD304. Human MSCs may be characterized by the absence of the cell-surface expression of markers (e.g. hematopoietic markers), including but not limited to, CD31, CD34, CD20, CD19, CD14 and CD45. According to one embodiment, human MSCs may be characterized by the absence of the cell-surface expression of CD14 and/or CD11b; CD19 and/or CD79a; or HLA-DR. It will be appreciated that all of the markers may be expressed on a single MSC cell, or alternatively, the markers may be expressed on different MSC cells (e.g. in the same culture).

According to a specific embodiment, human MSCs are characterized by the cell-surface expression of CD105, CD146, CD90, CD44, and the absence of cell-surface expression of CD31, CD34 and CD45 (i.e. are CD105⁺/CD146⁺/CD90⁺/CD44⁺/CD31⁻/CD34⁻/CD45⁻ cells).

MSCs may be obtained using any method known in the art. For example, MSCs can be obtained from bone marrow using standard procedures. For example, bone marrow aspirates or biopsies can be collected from donors (e.g. healthy donors) and MSCs can be isolated therefrom (see, for example, Aggarwal & Pittenger, Blood (2005) 105: 1815-1822). Generally, mononuclear cells are typically isolated from bone marrow aspirates by gradient centrifugation, are then seeded into flasks containing MSC medium, such as Dulbecco's modified Eagle medium (DMEM)-low glucose supplemented with 10 mM L-glutamine and 10% fetal calf serum (FCS), and grown at 37° C. under a humidified 5% CO₂ atmosphere. Non-adherent cells are typically removed after 24 hours (e.g. by washing with PBS-HSA solution). The culture medium is changed every 4 days and after 2 weeks the cultures should be mostly confluent. MSCs are recovered using trypsin and re-plated as passage 1 cells. Cells can be kept in culture for at least 8 passages and tested routinely for the presence of MSC-associated surface molecules. Similarly, MSCs may be obtained from adipose tissue, cord blood and umbilical cord as discussed in detail in www(dot)irvinesci(dot)com/protocol-for-mesenchymal-stem-cell-isolation, incorporated herein by reference.

Commercially available mesenchymal stem cells can also be used with this aspect of the present invention. Human MSCs can be purchased from e.g. the ATCC (American Type Culture Collection—www(dot)atcc(dot)org). Non-limiting examples of commercially available MSCs include adipose-derived mesenchymal stem cells e.g. ATCC® PCS-210-010™, ATCC® PCS-500-011™; bone marrow-derived mesenchymal stem cells e.g. ATCC® PCS-500-012™; umbilical cord-derived mesenchymal stem cells e.g. ATCC® PCS-500-010™.

According to one embodiment, the MSCs are obtained from PSCs (i.e. PSC-derived MSCs), e.g. differentiated from ESCs (e.g. hESCs) or from iPS cells, as further discussed below.

According to one embodiment, the pluripotent stem cells (PSCs) and/or mesenchymal stem cells (MSCs) of the invention are mammalian cells, including but not limited to, cells of a human, a primate, a dog, a horse, a cat, a cow, a pig, a mouse, a rat, a rabbit origin. According to a specific embodiment, the PSCs or MSCs are from a human origin.

As used herein the phrase “culture medium” refers to a liquid substance used to support the growth of cells. The culture medium used by the invention according to some embodiments can be a water-based medium which includes a combination of substances such as salts, nutrients, minerals, vitamins, amino acids, nucleic acids, proteins such as cytokines, growth factors and hormones, all of which are needed for cell proliferation and/or differentiation.

For example, a culture medium according to an aspect of some embodiments of the invention can be a synthetic tissue culture medium comprising a basal medium such as the Dulbecco's Modified Eagle's Medium (DMEM, available for example from Gibco-Invitrogen Corporation products, Grand Island, N.Y., USA), DMEM/F12 (available for example from Biological Industries, Beit HaEmek, Israel), DMEM high glucose (DMEM HG, available for example from Biological Industries, Beit HaEmek, Israel), MEM alpha (available for example from Biological Industries, Beit HaEmek, Israel), Ham's F-12 (available for example from Invitrogen/Thermo Fisher Scientific), Ko-DMEM (available for example from Gibco-Invitrogen Corporation products, Grand Island, N.Y., USA), Eagle's Minimum Essential Medium (EMEM, available for example from Gibco-Invitrogen Corporation products, Grand Island, N.Y., USA) supplemented with the necessary additives as is further described herein under. The concentration of the basal medium depends on the concentration of the other medium ingredients such as the serum replacement as discussed below.

According to one embodiment of the invention, the culture medium comprises the basal medium DMEM/F12.

According to one embodiment of the invention, the culture medium comprises the basal medium DMEM high glucose (DMEM HG).

According to one embodiment of the invention, the culture medium comprises the basal medium MEM alpha.

According to one embodiment of the invention, the culture medium comprises the basal media DMEM/F12 and MEM alpha.

According to one embodiment, the volume ratio of DMEM/F12 to MEM alpha ranges between 0.1 to 9 DMEM/F12 to MEM alpha (e.g. volume of 10% DMEM/F12 and 90% MEM alpha to 90% DMEM/F12 and 10% MEM alpha).

According to one embodiment, the volume ratio of DMEM/F12 to MEM alpha ranges between 0.25 to 4 DMEM/F12 to MEM alpha (e.g. volume of 20% DMEM/F12 and 80% MEM alpha to 80% DMEM/F12 and 20% MEM alpha).

According to one embodiment, the volume ratio of DMEM/F12 to MEM alpha ranges between 0.4 to 2.3 DMEM/F12 to MEM alpha (e.g. volume of 30% DMEM/F12 and 70% MEM alpha to 70% DMEM/F12 and 30% MEM alpha).

According to one embodiment, the volume ratio of DMEM/F12 to MEM alpha ranges between 0.6 to 1.5 DMEM/F12 to MEM alpha (e.g. volume of 40% DMEM/F12 and 60% MEM alpha to 60% DMEM/F12 and 40% MEM alpha).

According to one embodiment, the volume ratio of DMEM/F12 to MEM alpha is 1 to 1 DMEM/F12 to MEM alpha (e.g. volume of 50% DMEM/F12 and 50% MEM alpha).

According to one embodiment the culture medium is defined. A “defined” culture medium refers to a chemically-defined culture medium manufactured from known components at specific concentrations. For example, a defined culture medium is a non-conditioned culture medium.

A “conditioned culture medium” refers to a culture medium in which a specific cell or population of cells has been cultured, and then removed. When cells are cultured in a medium, they may secrete cellular factors that can provide trophic support to other cells. Such factors include, but are not limited to, growth factors, inflammatory mediators and other extracellular proteins, including e.g. hormones, cytokines, antibodies, extracellular matrix (ECM), vesicles, and granules. The medium containing the cellular factors is the conditioned medium (e.g. growth medium in which feeder cells, e.g. fibroblasts, have grown).

According to one embodiment, the culture medium is a non-conditioned culture medium.

According to one embodiment, the culture medium does not comprise components of a conditioned medium.

According to one embodiment, the culture medium is serum-free.

As used herein the phrase “serum-free” refers to being devoid of a human or an animal serum.

It should be noted that the function of serum in culturing protocols is to provide the cultured cells with an environment similar to that present in vivo (i.e., within the organism from which the cells are derived). However, the use of serum, which is derived from either an animal source (e.g., bovine serum) or a human source (human serum), is limited by the significant variations in serum components between the donor individuals (from which the serum is obtained) and the risk of having xeno contaminants (in case of an animal serum is used).

According to some embodiments of the invention, the serum-free culture medium does not comprise serum or portions thereof.

According to some embodiments of the invention, the serum-free culture medium is devoid of serum albumin (e.g., albumin which is purified from human serum or animal serum).

According to some embodiments of the invention the culture medium comprises serum replacement.

As used herein the phrase “serum replacement” refers to a defined formulation, which substitutes the function of serum by providing pluripotent stem cells or mesenchymal stem cells with components needed for growth and viability.

Various serum replacement formulations are known in the art and are commercially available.

For example, GIBCO™ Knockout™ Serum Replacement (KoSR, Gibco-Invitrogen Corporation, Grand Island, N.Y. USA, Catalogue No. 10828-028) is a defined serum-free formulation optimized to grow and maintain cells in culture. It should be noted that the formulation of GIBCO™ Knockout™ Serum Replacement includes Albumax (Bovine serum albumin enriched with lipids) which is from an animal source (International Patent Publication No. WO 98/30679 to Price, P. J. et al). However, a recent publication by Crook et al., 2007 (Crook J M., et al., 2007, Cell Stem Cell, 1: 490-494) describes six clinical-grade hESC lines generated using FDA-approved clinical grade foreskin fibroblasts in cGMP-manufactured Knockout™ Serum Replacement (Invitrogen Corporation, USA, Catalogue No. 04-0095).

According to some embodiments of the invention, the concentration of GIBCO™ Knockout™ Serum Replacement (KoSR) in the culture medium is in the range of from about 0.5% [volume/volume (v/v)] to about 50% (v/v), about 1% [volume/volume (v/v)] to about 50% (v/v), e.g., from about 1% (v/v) to about 5% (v/v), e.g., from about 1% (v/v) to about 10% (v/v), e.g., from about 3% (v/v) to about 5% (v/v), e.g., from about 3% (v/v) to about 10% (v/v), e.g., from about 3% (v/v) to about 15% (v/v), e.g., from about 5% (v/v) to about 10% (v/v), e.g., from about 5% (v/v) to about 15% (v/v), e.g., from about 5% (v/v) to about 30% (v/v), e.g., from about 5% (v/v) to about 40% (v/v), e.g., from about 10% (v/v) to about 15% (v/v), e.g., from about 10% (v/v) to about 20% (v/v).

According to some embodiments of the invention, the concentration of GIBCO™ Knockout™ Serum Replacement (KoSR) in the culture medium is at least e.g. about 0.5% (v/v), at least e.g. about 1% (v/v), at least e.g. about 1.5% (v/v), at least e.g. about 2% (v/v), at least e.g. about 2.5% (v/v), at least e.g. about 3% (v/v), at least e.g. about 3.5% (v/v), at least e.g. about 4% (v/v), at least e.g. about 5% (v/v), at least e.g. about 6% (v/v), at least e.g. about 7% (v/v), at least e.g. about 8% (v/v), at least e.g. about 9% (v/v), at least e.g. about 10% (v/v), at least e.g. about 11% (v/v), at least e.g. about 12% (v/v), at least e.g. about 13% (v/v), at least e.g. about 14% (v/v), at least e.g. about 15% (v/v), at least e.g. about 20% (v/v), at least e.g. about 30% (v/v).

According to some embodiments of the invention, the concentration of GIBCO™ Knockout™ Serum Replacement (KoSR) in the culture medium is at most e.g. about 10% (v/v), e.g. about 11% (v/v), e.g. about 12% (v/v), e.g. about 13% (v/v), e.g. about 14% (v/v), e.g. about 15% (v/v), e.g. about 20% (v/v), e.g. about 30% (v/v).

According to a specific embodiment, the concentration of GIBCO™ Knockout™ Serum Replacement (KoSR) in the culture medium is about 2.5% (v/v).

According to a specific embodiment, the concentration of GIBCO™ Knockout™ Serum Replacement (KoSR) in the culture medium is about 3% (v/v).

According to a specific embodiment, the concentration of GIBCO™ Knockout™ Serum Replacement (KoSR) in the culture medium is about 3.75% (v/v).

According to a specific embodiment, the concentration of GIBCO™ Knockout™ Serum Replacement (KoSR) in the culture medium is about 5% (v/v).

According to a specific embodiment, the concentration of GIBCO™ Knockout™ Serum Replacement (KoSR) in the culture medium is about 7.5% (v/v).

According to a specific embodiment, the concentration of GIBCO™ Knockout™ Serum Replacement (KoSR) in the culture medium is about 10% (v/v).

According to a specific embodiment, the concentration of GIBCO™ Knockout™ Serum Replacement (KoSR) in the culture medium is about 15% (v/v).

Another commercially available serum replacement is the B27 supplement without vitamin A which is available from e.g. Gibco-Invitrogen, Corporation, Grand Island, N.Y. USA, Catalogue No. 12587-010. The B27 supplement is a serum-free formulation which includes d-biotin, fatty acid free fraction V bovine serum albumin (BSA), catalase, L-carnitine HCl, corticosterone, ethanolamine HCl, D-galactose (Anhyd.), glutathione (reduced), recombinant human insulin, linoleic acid, linolenic acid, progesterone, putrescine-2-HCl, sodium selenite, superoxide dismutase, T-3/albumin complex, DL alpha-tocopherol and DL alpha tocopherol acetate. However, the use of B27 supplement is limited since it includes albumin from an animal source.

According to some embodiments of the invention, the serum replacement is xeno-free.

The term “xeno” is a prefix based on the Greek word “Xenos”, i.e., a stranger. As used herein the phrase “xeno-free” refers to being devoid of any components which are derived from a xenos (i.e., not the same, a foreigner) species. Such components can be contaminants such as pathogens associated with (e.g., infecting) the xeno species, cellular components of the xeno species or a-cellular components (e.g., fluid) of the xeno species.

For example, a xeno-free serum replacement can include a combination of insulin, transferrin and selenium. Additionally or alternatively, a xeno-free serum replacement can include human or recombinantly produced albumin, transferrin and insulin.

Non-limiting examples of commercially available xeno-free serum replacement compositions include the premix of ITS (Insulin, Transferrin and Selenium) available from e.g. Invitrogen corporation (ITS, Invitrogen, Catalogue No. 51500-056 or Gibco Catalogue No. 41400-045) and Serum replacement 3 (available from e.g. Sigma, Catalogue No. S2640) which includes human serum albumin, human transferring and human recombinant insulin and does not contain growth factors, steroid hormones, glucocorticoids, cell adhesion factors, detectable Ig and mitogens.

According to some embodiments of the invention, the concentration of ITS (Insulin, Transferrin and Selenium) in the culture medium is in the range of from about 0.01% [volume/volume (v/v)] to about 10% (v/v), e.g., from about 0.01% (v/v) to about 0.05% (v/v), e.g., from about 0.01% (v/v) to about 0.1% (v/v), e.g., from about 0.01% (v/v) to about 0.5% (v/v), e.g., from about 0.01% (v/v) to about 1% (v/v), e.g., from about 0.01% (v/v) to about 3% (v/v), e.g., from about 0.01% (v/v) to about 5% (v/v), e.g., from about 0.1% (v/v) to about 0.5% (v/v), e.g., from about 0.1% (v/v) to about 0.8% (v/v), e.g., from about 0.1% (v/v) to about 1.0% (v/v), e.g., from about 0.1% (v/v) to about 1.5% (v/v), e.g., from about 0.1% (v/v) to about 2% (v/v), e.g., from about 0.1% (v/v) to about 3% (v/v), e.g., from about 0.1% (v/v) to about 5% (v/v), e.g., from about 0.2% (v/v) to about 0.3% (v/v), e.g., from about 0.2% (v/v) to about 0.4% (v/v), e.g., from about 0.2% (v/v) to about 0.5% (v/v), e.g., from about 0.5% (v/v) to about 1% (v/v), e.g., from about 0.5% (v/v) to about 1.5% (v/v), e.g., from about 0.5% (v/v) to about 2% (v/v), e.g., from about 0.5% (v/v) to about 3% (v/v), e.g., from about 0.5% (v/v) to about 5% (v/v), e.g., from about 0.5% (v/v) to about 10% (v/v), e.g., from about 1% (v/v) to about 2% (v/v), e.g., from about 1% (v/v) to about 3% (v/v), e.g., from about 1% (v/v) to about 5% (v/v), e.g., from about 1% (v/v) to about 10% (v/v).

According to some embodiments of the invention, the concentration of ITS (Insulin, Transferrin and Selenium) in the culture medium is at least e.g. about 0.01% (v/v), at least e.g. about 0.05% (v/v), at least e.g. about 0.1% (v/v), at least e.g. about 0.2% (v/v), at least e.g. about 0.3% (v/v), at least e.g. about 0.4% (v/v), at least e.g. about 0.5% (v/v), at least e.g. about 0.6% (v/v), at least e.g. about 0.7% (v/v), at least e.g. about 0.8% (v/v), at least e.g. about 0.9% (v/v), at least e.g. about 1% (v/v), at least e.g. about 1.5% (v/v), at least e.g. about 2% (v/v), at least e.g. about 2.5% (v/v), at least e.g. about 3% (v/v), at least e.g. about 3.5% (v/v), at least e.g. about 4% (v/v), at least e.g. about 5% (v/v), at least e.g. about 6% (v/v), at least e.g. about 7% (v/v), at least e.g. about 8% (v/v), at least e.g. about 9% (v/v), at least e.g. about 10% (v/v).

According to some embodiments of the invention, the concentration of ITS (Insulin, Transferrin and Selenium) in the culture medium is at most e.g. about 0.2%, e.g. about 0.3%, e.g. about 0.5% (v/v), e.g. about 1% (v/v), e.g. about 1.5% (v/v), e.g. about 2% (v/v), e.g. about 2.5% (v/v), e.g. about 3% (v/v), e.g. about 4% (v/v), e.g. about 5% (v/v), e.g. about 6% (v/v), e.g. about 7% (v/v), e.g. about 8% (v/v), e.g. about 9% (v/v), e.g. about 10% (v/v).

According to a specific embodiment, the concentration of ITS (Insulin, Transferrin and Selenium) in the culture medium is about 0.1% (v/v).

According to a specific embodiment, the concentration of ITS (Insulin, Transferrin and Selenium) in the culture medium is about 0.25% (v/v).

According to a specific embodiment, the concentration of ITS (Insulin, Transferrin and Selenium) in the culture medium is about 0.5% (v/v).

According to a specific embodiment, the concentration of ITS (Insulin, Transferrin and Selenium) in the culture medium is about 1% (v/v).

According to a specific embodiment, the concentration of ITS (Insulin, Transferrin and Selenium) in the culture medium is about 2% (v/v).

According to a specific embodiment, the concentration of ITS (Insulin, Transferrin and Selenium) in the culture medium is about 3% (v/v).

According to one embodiment, the culture medium comprises GIBCO™ Knockout™ Serum Replacement (KoSR) and ITS (Insulin, Transferrin and Selenium).

According to one embodiment, the culture medium comprises the basal medium DMEM/F12, GIBCO™ Knockout™ Serum Replacement (KoSR) and ITS (Insulin, Transferrin and Selenium).

According to one embodiment, the culture medium comprising the basal medium DMEM/F12, GIBCO™ Knockout™ Serum Replacement (KoSR) and ITS (Insulin, Transferrin and Selenium) is serum-free.

According to one embodiment, the culture medium comprises the basal media DMEM/F12 and MEM alpha, GIBCO™ Knockout™ Serum Replacement (KoSR) and ITS (Insulin, Transferrin and Selenium).

According to one embodiment, the culture medium comprising the basal media MEM alpha and ITS (Insulin, Transferrin and Selenium).

According to one embodiment, the culture medium comprises serum, i.e. an undefined mixture of different soluble proteins and growth factors, which support the survival and proliferation of cells. Various serum formulations are known in the art and are commercially available. For example, Fetal bovine serum (FBS, Biological Industries, Beit HaEmek, Israel, Cat no. 04-007-1A), Human AB Serum, Porcine serum, Horse serum, Rabbit serum and Goat serum, all of which are commercially available from e.g. Biological Industries, Beit HaEmek, Israel.

According to some embodiments of the invention, the serum (e.g. FBS) in the culture medium is in the range of from about 1% [volume/volume (v/v)] to about 50% (v/v), e.g., from about 1% (v/v) to about 5% (v/v), e.g., from about 1% (v/v) to about 10% (v/v), e.g., from about 1% (v/v) to about 20% (v/v), e.g., from about 1% (v/v) to about 30% (v/v), e.g., from about 3% (v/v) to about 30% (v/v), e.g., from about 5% (v/v) to about 10% (v/v), e.g., from about 5% (v/v) to about 15% (v/v), e.g., from about 5% (v/v) to about 20% (v/v), e.g., from about 5% (v/v) to about 30% (v/v), e.g., from about 5% (v/v) to about 40% (v/v), e.g., from about 10% (v/v) to about 20% (v/v), e.g., from about 10% (v/v) to about 30% (v/v), e.g., from about 10% (v/v) to about 40% (v/v), e.g., from about 10% (v/v) to about 50% (v/v), e.g., from about 20% (v/v) to about 30% (v/v), e.g., from about 20% (v/v) to about 40% (v/v), e.g., from about 20% (v/v) to about 50% (v/v).

According to some embodiments of the invention, the serum (e.g. FBS) in the culture medium is at least e.g. about 1% (v/v), at least e.g. about 2% (v/v), at least e.g. about 3% (v/v), at least e.g. about 4% (v/v), at least e.g. about 5% (v/v), at least e.g. about 6% (v/v), at least e.g. about 7% (v/v), at least e.g. about 8% (v/v), at least e.g. about 9% (v/v), at least e.g. about 10% (v/v), at least e.g. about 15% (v/v), at least e.g. about 20% (v/v), at least e.g. about 25% (v/v), at least e.g. about 30% (v/v), at least e.g. about 35% (v/v), at least e.g. about 40% (v/v).

According to some embodiments of the invention, the serum (e.g. FBS) in the culture medium is at most e.g. about 5% (v/v), e.g. about 10% (v/v), e.g. about 15% (v/v), e.g. about 20% (v/v), e.g. about 25% (v/v), e.g. about 30% (v/v), e.g. about 35% (v/v), e.g. about 40% (v/v), e.g. about 50% (v/v).

According to some embodiments of the invention, the serum (e.g. FBS) in the culture medium is about 3% (v/v).

According to some embodiments of the invention, the serum (e.g. FBS) in the culture medium is about 5% (v/v).

According to some embodiments of the invention, the serum (e.g. FBS) in the culture medium is about 10% (v/v).

According to some embodiments of the invention, the serum (e.g. FBS) in the culture medium is about 20% (v/v).

According to some embodiments of the invention, the serum (e.g. FBS) in the culture medium is about 30% (v/v).

According to one embodiment, the culture medium comprises the basal media DMEM/F12 and MEM alpha, GIBCO™ Knockout™ Serum Replacement (KoSR), ITS (Insulin, Transferrin and Selenium) and serum.

According to one embodiment, the culture medium comprises the basal media MEM alpha, ITS (Insulin, Transferrin and Selenium) and serum.

According to some embodiments of the invention, the culture medium comprises glucose. Glucose can be obtained from various suppliers such as, but not limited to, Sigma-Aldrich.

The concentration of glucose in the culture medium can be from about 0.5 gr/Liter-5 gr/Liter, e.g., 1 gr/Liter-5 gr/Liter, e.g., 1 gr/Liter-4.5 gr/Liter, e.g. 1 gr/Liter-4 gr/Liter, e.g. 1 gr/Liter-3.5 gr/Liter, e.g. 1 gr/Liter-3 gr/Liter.

According to some embodiments of the invention, the glucose in the culture medium is at least e.g. about 0.5 gr/Liter, e.g. about 1 gr/Liter, e.g. about 1.5 gr/Liter, e.g. about 2 gr/Liter, e.g. about 2.5 gr/Liter, e.g. about 3 gr/Liter, e.g. about 3.5 gr/Liter, e.g. about 4 gr/Liter, e.g. about 4.5 gr/Liter, e.g. about 5 gr/Liter.

According to some embodiments of the invention, the glucose in the culture medium is at most e.g. about 5 gr/Liter, e.g. about 4.5 gr/Liter, e.g. about 4 gr/Liter, e.g. about 3.5 gr/Liter.

According to some embodiments of the invention, the glucose in the culture medium is about 3.5 gr/Liter.

According to some embodiments of the invention, the glucose in the culture medium is about 4 gr/Liter.

According to some embodiments of the invention, the glucose in the culture medium is about 4.5 gr/Liter.

According to some embodiments of the invention, the glucose in the culture medium is about 5 gr/Liter.

According to some embodiments of the invention, the culture medium comprises L-ascorbic acid. L-ascorbic acid can be obtained from various suppliers such as, but not limited to, Sigma-Aldrich.

The concentration of L-ascorbic acid in the culture medium can be from about 20 μg/ml-200 μg/ml, e.g. 20 μg/ml-100 μg/ml, e.g. 20 μg/ml-60 μg/ml, e.g., 50 μg/ml-200 μg/ml, e.g. 50 μg/ml-150 μg/ml, e.g. 50 μg/ml-100 μg/ml, e.g. 50 μg/ml-75 μg/ml.

According to some embodiments of the invention, the L-ascorbic acid in the culture medium is at least e.g. about 20 μg/ml, e.g. about 30 μg/ml, e.g. about 40 μg/ml, e.g. about 50 μg/ml, e.g. about 60 μg/ml, e.g. about 70 μg/ml, e.g. about 80 μg/ml, e.g. about 90 μg/ml, e.g. about 100 μg/ml, e.g. about 120 μg/ml, e.g. about 140 μg/ml, e.g. about 160 μg/ml, e.g. about 180 μg/ml, e.g. about 200 μg/ml.

According to some embodiments of the invention, the L-ascorbic acid in the culture medium is at most e.g. about 200 μg/ml, e.g. about 175 μg/ml, e.g. about 150 μg/ml, e.g. about 100 μg/ml, e.g. about 75 μg/ml, e.g. about 50 μg/ml.

According to some embodiments of the invention, the L-ascorbic acid in the culture medium is about 25 μg/ml.

According to some embodiments of the invention, the L-ascorbic acid in the culture medium is about 50 μg/ml.

According to some embodiments of the invention, the L-ascorbic acid in the culture medium is about 75 μg/ml.

According to some embodiments of the invention, the L-ascorbic acid in the culture medium is about 100 μg/ml.

According to some embodiments of the invention, the culture medium comprises Sodium pyruvate. Sodium pyruvate can be obtained from various suppliers such as, but not limited to, Biological Industries, Beit HaEmek, Israel.

The concentration of Sodium pyruvate in the culture medium can be from about 20 μg/ml-200 μg/ml, e.g. 20 μg/ml-100 μg/ml, e.g. 20 μg/ml-60 μg/ml, e.g., 50 μg/ml-200 μg/ml, e.g. 50 μg/ml-150 μg/ml, e.g. 50 μg/ml-100 μg/ml, e.g. 50 μg/ml-75 μg/ml.

According to some embodiments of the invention, the Sodium pyruvate in the culture medium is at least e.g. about 20 μg/ml, e.g. about 30 μg/ml, e.g. about 40 μg/ml, e.g. about 50 μg/ml, e.g. about 60 μg/ml, e.g. about 70 μg/ml, e.g. about 80 μg/ml, e.g. about 90 μg/ml, e.g. about 100 μg/ml, e.g. about 120 μg/ml, e.g. about 140 μg/ml, e.g. about 160 μg/ml, e.g. about 180 μg/ml, e.g. about 200 μg/ml.

According to some embodiments of the invention, the Sodium pyruvate in the culture medium is at most e.g. about 200 μg/ml, e.g. about 175 μg/ml, e.g. about 150 μg/ml, e.g. about 100 μg/ml, e.g. about 75 μg/ml, e.g. about 50 μg/ml.

According to some embodiments of the invention, the Sodium pyruvate in the culture medium is about 25 μg/ml.

According to some embodiments of the invention, the Sodium pyruvate in the culture medium is about 50 μg/ml.

According to some embodiments of the invention, the Sodium pyruvate in the culture medium is about 75 μg/ml.

According to some embodiments of the invention, the Sodium pyruvate in the culture medium is about 100 μg/ml.

According to one embodiment, the culture medium comprises the basal media DMEM/F12, KoSR, ITS (Insulin, Transferrin and Selenium) and L-ascorbic acid.

According to one embodiment, the culture medium comprises the basal media DMEM/F12, KoSR, ITS (Insulin, Transferrin and Selenium), L-ascorbic acid and Sodium pyruvate.

According to one embodiment, the culture medium comprises the basal media DMEM/F12, KoSR, ITS (Insulin, Transferrin and Selenium), glucose, L-ascorbic acid and Sodium pyruvate.

According to one embodiment, the culture medium comprises the basal media DMEM high glucose (DMEM HG), KoSR, ITS (Insulin, Transferrin and Selenium), L-ascorbic acid and Sodium pyruvate.

As is shown in Examples 1 and 2 of the Examples section which follows, the present inventors have used various culture media which include bFGF in the range of e.g. 1-50 ng/ml (as depicted in Tables 2 and 3, herein below) to successfully differentiate PSCs into MSCs, and to successfully culture MSCs and maintain them in a proliferative, multipotent and undifferentiated state (as further discussed below).

According to some embodiments of the invention the culture medium comprises basic fibroblast growth factor (bFGF).

As used herein the term “basic fibroblast growth factor” or “bFGF” refers to a polypeptide of the fibroblast growth factor (FGF) family, which binds heparin and possesses broad mitogenic and angiogenic activities. The mRNA for the BFGF gene contains multiple polyadenylation sites, and is alternatively translated from non-AUG (CUG) and AUG initiation codons, resulting in five different isoforms with distinct properties. The CUG-initiated isoforms are localized in the nucleus and are responsible for the intracrine effect, whereas, the AUG-initiated form is mostly cytosolic and is responsible for the paracrine and autocrine effects of this FGF.

According to some embodiments of the invention, the basic fibroblast growth factor (bFGF) comprises FGF2 or FGF4.

FGF2, also generally known as basic fibroblast growth factor (FGF basic, bFGF or FGF-β) is a member of the fibroblast growth factor family. The FGF2 used in the culture medium of some embodiments of the invention can be a purified, a synthetic or a recombinantly expressed FGF2 protein (e.g., human bFGF polypeptide GenBank Accession No. NP_001997.5; e.g., human bFGF polynucleotide GenBank Accession No. NM_002006).

FGF4 is a member of the fibroblast growth factor family. The FGF4 used in the culture medium of some embodiments of the invention can be a purified, a synthetic or a recombinantly expressed FGF4 protein (e.g., human bFGF polypeptide GenBank Accession No. NP_001998.1; e.g., human bFGF polynucleotide GenBank Accession No. NM_002007).

According to a specific embodiment, the basic fibroblast growth factor (bFGF) comprises the protein encoded by the gene comprising the symbol FGF2 or FGF4.

It should be noted that for the preparation of a xeno-free culture medium the bFGF (e.g. FGF2 and/or FGF4) is preferably purified from a human source or is recombinantly expressed as is further described hereinbelow. bFGF (e.g. FGF2 and/or FGF4) can be obtained from various commercial sources such as, for example, from Biological Industries (Beit HaEmek, Israel), BioLegend, San Diego, Calif., USA (Catalogue Nos. 710304/8 and 710404, respectively). According to one embodiment, bFGF (i.e. FGF2) is obtained from R&D Systems, Cat No: 233-FB, and FGF4 is obtained from R&D Systems, Cat No: 235-F4/CF, or from Peprotech, Cat No: 100-31.

According to some embodiments the concentration of bFGF (e.g. FGF2 and/or FGF4) in culture medium is in the range from about 0.01 ng/ml to about 10 μg/ml, e.g., from about 0.1 ng/ml to about 10 μg/ml, e.g., from about 1 ng/ml to about 1 μg/ml, e.g., from about 1 ng/ml to about 10 ng/ml, e.g., from about 1 ng/ml to about 50 ng/ml, e.g., from about 1 ng/ml to about 100 ng/ml, e.g., from about 1 ng/ml to about 500 ng/ml, e.g., from about 2 ng/ml to about 200 ng/ml, e.g., from about 5 ng/ml to about 50 ng/ml, e.g., from about 5 ng/ml to about 100 ng/ml, e.g., from about 5 ng/ml to about 150 ng/ml, e.g., from about 5 ng/ml to about 200 ng/ml, e.g., from about 5 ng/ml to about 250 ng/ml, from about 5 ng/ml to about 500 ng/ml, e.g., from about 10 ng/ml to about 100 ng/ml, e.g., from about 10 ng/ml to about 150 ng/ml, e.g., from about 10 ng/ml to about 200 ng/ml, e.g., from about 10 ng/ml to about 250 ng/ml, from about 10 ng/ml to about 500 ng/ml, e.g., from about 50 ng/ml to about 100 ng/ml, e.g., from about 50 ng/ml to about 150 ng/ml, e.g., from about 50 ng/ml to about 200 ng/ml, e.g., from about 50 ng/ml to about 250 ng/ml, from about 50 ng/ml to about 500 ng/ml.

According to some embodiments of the invention, the concentration of bFGF (e.g. FGF2 and/or FGF4) in the culture medium is at least e.g. about 0.01 ng/ml, at least e.g. about 0.1 ng/ml, at least e.g. about 0.5 ng/ml, at least e.g. about 1 ng/ml, at least e.g. about 1.5 ng/ml, at least e.g. about 2 ng/ml, at least e.g. about 2.5 ng/ml, at least e.g. about 3 ng/ml, at least e.g. about 4 ng/ml, at least e.g. about 5 ng/ml, at least e.g. about 7.5 ng/ml, at least e.g. about 10 ng/ml, at least e.g. about 15 ng/ml, at least e.g. about 20 ng/ml, at least e.g. about 25 ng/ml, at least e.g. about 30 ng/ml, at least e.g. about 40 ng/ml, at least e.g. about 50 ng/ml, at least e.g. about 60 ng/ml, at least e.g. about 70 ng/ml, at least e.g. about 80 ng/ml, at least e.g. about 90 ng/ml, at least e.g. about 100 ng/ml, at least e.g. about 120 ng/ml, at least e.g. about 130 ng/ml, at least e.g. about 140 ng/ml, at least e.g. about 150 ng/ml.

According to some embodiments of the invention the concentration of bFGF (e.g. FGF2 and/or FGF4) in the culture medium is at most about 5 ng/ml, at most about 10 ng/ml, at most about 15 ng/ml, at most about 20 ng/ml, at most about 30 ng/ml, at most about 40 ng/ml, at most about 50 ng/ml, at most about 60 ng/ml, at most about 70 ng/ml, at most about 80 ng/ml, at most about 90 ng/ml, at most about 100 ng/ml, at most about 200 ng/ml, at most about 500 ng/ml.

According to one embodiment, the culture medium comprises FGF2.

According to one embodiment, the culture medium comprises FGF4.

According to one embodiment, the culture medium comprises FGF2 and FGF4.

According to a specific embodiment, the concentration of FGF2 in the culture medium is about 1 ng/ml.

According to a specific embodiment, the concentration of FGF2 in the culture medium is about 5 ng/ml.

According to a specific embodiment, the concentration of FGF2 in the culture medium is about 10 ng/ml.

According to a specific embodiment, the concentration of FGF2 in the culture medium is about 20 ng/ml.

According to a specific embodiment, the concentration of FGF2 in the culture medium is about 50 ng/ml.

According to a specific embodiment, the concentration of FGF4 in the culture medium is about 1 ng/ml.

According to a specific embodiment, the concentration of FGF4 in the culture medium is about 5 ng/ml.

According to a specific embodiment, the concentration of FGF4 in the culture medium is about 10 ng/ml.

According to a specific embodiment, the concentration of FGF4 in the culture medium is about 20 ng/ml.

According to a specific embodiment, the concentration of FGF4 in the culture medium is about 50 ng/ml.

According to one embodiment, the culture medium comprises the basal medium DMEM/F12, GIBCO™ Knockout™ Serum Replacement (KoSR), ITS (Insulin, Transferrin and Selenium) and FGF2. According to a specific embodiment, this culture medium is serum-free.

According to one embodiment, the culture medium comprises the basal medium DMEM/F12, GIBCO™ Knockout™ Serum Replacement (KoSR), ITS (Insulin, Transferrin and Selenium) and FGF4. According to a specific embodiment, this culture medium is serum-free.

According to one embodiment, the culture medium comprises the basal medium DMEM/F12, GIBCO™ Knockout™ Serum Replacement (KoSR), ITS (Insulin, Transferrin and Selenium), FGF2 and FGF4. According to a specific embodiment, this culture medium is serum-free.

According to one embodiment, the culture medium comprises the basal media DMEM/F12 and MEM alpha, GIBCO™ Knockout™ Serum Replacement (KoSR), ITS (Insulin, Transferrin and Selenium) and FGF2. According to a specific embodiment, this culture medium comprises serum.

According to one embodiment, the culture medium comprises the basal media DMEM/F12 and MEM alpha, GIBCO™ Knockout™ Serum Replacement (KoSR), ITS (Insulin, Transferrin and Selenium) and FGF4. According to a specific embodiment, this culture medium comprises serum.

According to one embodiment, the culture medium comprises the basal media DMEM/F12 and MEM alpha, GIBCO™ Knockout™ Serum Replacement (KoSR), ITS (Insulin, Transferrin and Selenium), FGF2 and FGF4. According to a specific embodiment, this culture medium comprises serum.

According to some embodiments of the invention the culture medium comprises transforming growth factor beta (TGFβ).

As used herein the term “transforming growth factor beta” or “TGFβ” refers to any isoform of the transforming growth factor beta (β), which functions through the same receptor signaling system in the control of proliferation, differentiation, and other functions in many cell types. TGFβ acts in inducing transformation and also acts as a negative autocrine growth factor.

According to some embodiments of the invention the TGFβ comprises TGFβ 1 and/or TGFβ3.

Transforming growth factor beta-1 (TGFβ₁) is a polypeptide member of the transforming growth factor beta superfamily of cytokines. TGFβ₁ is typically involved in the control of cell growth, proliferation, differentiation, and apoptosis in many cell types.

The TGFβ₁ used in the culture medium of some embodiments of the invention can be a purified, a synthetic or a recombinantly expressed TGFβ₁ protein (e.g., human TGFβ₁ polypeptide GenBank Accession No. NP_000651.3; e.g., human TGFβ₁ polynucleotide GenBank Accession No. NM_000660). TGFβ₁ can be obtained from various commercial sources such as RayBiotech, BioLegend and ProSci Proteins. According to a specific embodiment, TGFβ₁ is obtained from R&D systems, Cat. No.: 240-B/CF, or from Peprotech, Cat No: 100-21.

Transforming growth factor beta-3 (TGFβ₃) is a polypeptide member of the transforming growth factor beta superfamily of cytokines. TGFβ₃ is typically involved in the control of proliferation, differentiation, and other functions in many cell types, acts in inducing transformation and as a negative autocrine growth factor.

The TGFβ₃ used in the culture medium of some embodiments of the invention can be a purified, a synthetic or a recombinantly expressed TGFβ₃ protein (e.g., human TGFβ₃ polypeptide GenBank Accession No. NP_003230.1; e.g., human TGFβ₃ polynucleotide GenBank Accession No. NM_003239). TGFβ₃ can be obtained from various commercial sources such as Abcam Proteins, BioLegend and ProSci Proteins. According to a specific embodiment, TGFβ₃ is obtained from R&D systems, Cat. No.: 243-B3, or from Peprotech, Cat No: 100-36E.

According to a specific embodiment, the transforming growth factor beta comprises the protein encoded by the gene comprising the symbol TGFβ1 or TGFβ3.

It should be noted that for the preparation of a xeno-free culture medium the TGFβ (e.g. TGFβ₁ and/or TGFβ₃) is preferably purified from a human source or is recombinantly expressed as is further described hereinbelow.

According to some embodiments of the invention, the concentration of TGFβ₁ and/or TGFβ3 in the culture medium is in the range of about 0.05 ng/ml to about 1 μg/ml, e.g., from about 0.1 ng/ml to about 1 μg/ml, e.g. from about 0.5 ng/ml to about 100 ng/ml, e.g. from about 1 ng/ml to about 100 ng/ml, e.g. from about 1 ng/ml to about 50 ng/ml, e.g. from about 5 ng/ml to about 50 ng/ml, e.g. from about 5 ng/ml to about 10 ng/ml.

According to some embodiments of the invention, the concentration of TGFβ₁ and/or TGFβ3 in the culture medium is at least e.g. about 0.1 ng/ml, at least e.g. about 0.3 ng/ml, at least e.g. about 0.5 ng/ml, at least e.g. about 0.7 ng/ml, at least e.g. about 1 ng/ml, at least e.g. about 1.2 ng/ml, at least e.g. about 1.4 ng/ml, at least e.g. about 1.6 ng/ml, at least e.g. about 1.8 ng/ml, at least e.g. about 2 ng/ml, at least e.g. about 2.5 ng/ml, at least e.g. about 3 ng/ml, at least e.g. about 3.5 ng/ml, at least e.g. about 4 ng/ml at least e.g. about 5 ng/ml, at least e.g. about 6 ng/ml, at least e.g. about 7 ng/ml, at least e.g. about 8 ng/ml, at least e.g. about 9 ng/ml, at least e.g. about 10 ng/ml, at least e.g. about 11 ng/ml, at least e.g. about 12 ng/ml, at least e.g. about 13 ng/ml, at least e.g. about 14 ng/ml, at least e.g. about 15 ng/ml, at least e.g. about 20 ng/ml.

According to some embodiments of the invention, the concentration of TGFβ₁ and/or TGFβ3 in the culture medium is at most e.g. about 0.5 ng/ml, at most e.g. about 1 ng/ml, at most e.g. about 1.5 ng/ml, at most e.g. about 2 ng/ml, at most e.g. about 3 ng/ml, at most e.g. about 4 ng/ml, at most e.g. about 5 ng/ml, at most e.g. about 6 ng/ml, at most e.g. about 7 ng/ml, at most e.g. about 8 ng/ml, at most e.g. about 9 ng/ml, at most e.g. about 10 ng/ml, at most e.g. about 15 ng/ml, at most e.g. about 20 ng/ml.

According to a specific embodiment, the concentration of TGFβ₁ in the culture medium is about 1 ng/ml.

According to a specific embodiment, the concentration of TGFβ₁ in the culture medium is about 5 ng/ml.

According to a specific embodiment, the concentration of TGFβ₁ in the culture medium is about 10 ng/ml.

According to a specific embodiment, the concentration of TGFβ₁ in the culture medium is about 20 ng/ml.

According to a specific embodiment, the concentration of TGFβ₃ in the culture medium is about 1 ng/ml.

According to a specific embodiment, the concentration of TGFβ₃ in the culture medium is about 5 ng/ml.

According to a specific embodiment, the concentration of TGFβ₃ in the culture medium is about 10 ng/ml.

According to a specific embodiment, the concentration of TGFβ₃ in the culture medium is about 20 ng/ml.

According to one embodiment, the culture medium comprises the basal medium DMEM/F12, GIBCO™ Knockout™ Serum Replacement (KoSR), ITS (Insulin, Transferrin and Selenium) and TGFβ₁. According to a specific embodiment, this culture medium is serum-free. According to a specific embodiment, this culture medium comprises FGF2.

According to one embodiment, the culture medium comprises the basal medium DMEM/F12, GIBCO™ Knockout™ Serum Replacement (KoSR), ITS (Insulin, Transferrin and Selenium) and TGFβ₁. According to a specific embodiment, this culture medium is serum-free. According to a specific embodiment, this culture medium comprises FGF4.

According to one embodiment, the culture medium comprises the basal medium DMEM/F12, GIBCO™ Knockout™ Serum Replacement (KoSR), ITS (Insulin, Transferrin and Selenium) and TGFβ₁. According to a specific embodiment, this culture medium is serum-free. According to a specific embodiment, this culture medium comprises FGF2 and FGF4.

According to one embodiment, the culture medium comprises the basal medium DMEM/F12, GIBCO™ Knockout™ Serum Replacement (KoSR), ITS (Insulin, Transferrin and Selenium) and TGFβ₃. According to a specific embodiment, this culture medium is serum-free. According to a specific embodiment, this culture medium comprises FGF2.

According to one embodiment, the culture medium comprises the basal medium DMEM/F12, GIBCO™ Knockout™ Serum Replacement (KoSR), ITS (Insulin, Transferrin and Selenium) and TGFβ₃. According to a specific embodiment, this culture medium is serum-free. According to a specific embodiment, this culture medium comprises FGF4.

According to one embodiment, the culture medium comprises the basal medium DMEM/F12, GIBCO™ Knockout™ Serum Replacement (KoSR), ITS (Insulin, Transferrin and Selenium) and TGFβ₃. According to a specific embodiment, this culture medium is serum-free. According to a specific embodiment, this culture medium comprises FGF2 and FGF4.

According to one embodiment, the culture medium comprises the basal medium DMEM/F12, GIBCO™ Knockout™ Serum Replacement (KoSR), ITS (Insulin, Transferrin and Selenium) TGFβ₁ and TGFβ₃. According to a specific embodiment, this culture medium is serum-free. According to a specific embodiment, this culture medium comprises FGF2.

According to one embodiment, the culture medium comprises the basal medium DMEM/F12, GIBCO™ Knockout™ Serum Replacement (KoSR), ITS (Insulin, Transferrin and Selenium) TGFβ₁ and TGFβ₃. According to a specific embodiment, this culture medium is serum-free. According to a specific embodiment, this culture medium comprises FGF4.

According to one embodiment, the culture medium comprises the basal medium DMEM/F12, GIBCO™ Knockout™ Serum Replacement (KoSR), ITS (Insulin, Transferrin and Selenium) TGFβ₁ and TGFβ₃. According to a specific embodiment, this culture medium is serum-free. According to a specific embodiment, this culture medium comprises FGF2 and FGF4.

According to some embodiments of the invention the culture medium comprises WNT-3a.

As used herein the term “WNT-3A” refers to a member of the WNT gene family. The WNT gene family consists of structurally related genes which encode secreted signaling proteins. These proteins have been implicated in oncogenesis and in several developmental processes, including regulation of cell fate and patterning during embryogenesis.

The WNT-3A used in the culture medium of some embodiments of the invention can be a purified, a synthetic or a recombinantly expressed WNT-3A protein, e.g., GenBank Accession No. NP_149122.1, or WNT-3A polynucleotide e.g. GenBank Accession No. NM_033131.3). The WNT-3A polypeptide can be obtained from various manufacturers such as R&D SYSTEMS (e.g., Catalogue Nos. 5036-WN-010, 5036-WN/CF).

According to a specific embodiment, the WNT-3A comprises the protein encoded by the gene comprising the symbol WNT3A.

According to some embodiments of the invention, the culture medium further comprises a stabilizing agent of the WNT-3A, i.e. an agent which inhibits proteases and stabilizes the polypeptide WNT-3A. Examples of stabilizing agents include, but are not limited to, liposomes, micelles and the like, such as dimyristoyl-sn-Glycero-3-Phosphocholine (DMPC), 1,2-dimyristoyl-sn-glycero-3-phospho-(1′-rac-glycerol)) (DMPG) and cholesterol, e.g. in a DMPC:DMPG:cholesterol ration of 10:1:10.

According to some embodiments of the invention, the concentration of WNT-3A in the culture medium is in the range of about 0.1 ng/ml to about 1 μg/ml, e.g., from about 0.1 ng/ml to about 10 μg/ml, e.g. from about 0.5 ng/ml to about 100 ng/ml, e.g. from about 1 ng/ml to about 100 ng/ml, e.g. from about 10 ng/ml to about 200 ng/ml, e.g. from about 10 ng/ml to about 100 ng/ml, e.g. from about 50 ng/ml to about 200 ng/ml, e.g. from about 50 ng/ml to about 150 ng/ml.

According to some embodiments of the invention, the concentration of WNT-3A in the culture medium is at least e.g. about 0.5 ng/ml, e.g. at least about 1 ng/ml, e.g. at least about 5 ng/ml, e.g. at least about 10 ng/ml, e.g. at least about 15 ng/ml, e.g. at least about 20 ng/ml, e.g. at least about 25 ng/ml, e.g. at least about 30 ng/ml, e.g. at least about 40 ng/ml, e.g. at least about 50 ng/ml, e.g. at least about 60 ng/ml, e.g. at least about 70 ng/ml, e.g. at least about 80 ng/ml, e.g. at least about 90 ng/ml, e.g. at least about 100 ng/ml, e.g. at least about 120 ng/ml, e.g. at least about 140 ng/ml, e.g. at least about 160 ng/ml, e.g. at least about 180 ng/ml, e.g. at least about 200 ng/ml.

According to some embodiments of the invention, the concentration of WNT-3A in the culture medium is at most e.g. about 10 ng/ml, at most e.g. about 25 ng/ml, at most e.g. about 50 ng/ml, at most e.g. about 75 ng/ml, at most e.g. about 100 ng/ml, at most e.g. about 125 ng/ml, at most e.g. about 150 ng/ml, at most e.g. about 175 ng/ml, at most e.g. about 200 ng/ml.

According to a specific embodiment, the concentration of WNT-3A in the culture medium is about 10 ng/ml.

According to a specific embodiment, the concentration of WNT-3A in the culture medium is about 20 ng/ml.

According to a specific embodiment, the concentration of WNT-3A in the culture medium is about 200 ng/ml.

According to one embodiment, the culture medium comprises the basal medium DMEM/F12, GIBCO™ Knockout™ Serum Replacement (KoSR), ITS (Insulin, Transferrin and Selenium) and WNT-3A. According to a specific embodiment, this culture medium is serum-free. According to a specific embodiment, this culture medium comprises FGF2.

According to one embodiment, the culture medium comprises the basal medium DMEM/F12, GIBCO™ Knockout™ Serum Replacement (KoSR), ITS (Insulin, Transferrin and Selenium) and WNT-3A. According to a specific embodiment, this culture medium is serum-free. According to a specific embodiment, this culture medium comprises FGF2 and/or FGF4.

According to some embodiments of the invention the culture medium comprises platelet-derived growth factor (PDGF).

As used herein the term “Platelet-derived growth factor” or “PDGF” refers to any of four different isoforms of PDGF that activate cellular responses through two different receptors. Those isoforms include A (observed as a homodimer designated PDGF-AA and as part of a heterodimer with the B isoform designated PDGF-AB), B (observed as a homodomer designated PDGF-BB and as part of a heterodimer with the A isoform designated PDGF-AB), C (observed as a homodimer designated PDGF-CC) and D (observed as a homodimer designated PDGF-DD). Thus, the term “PDGF” as used herein refers generally to the known PDGF homo- and heterodimers (e.g., PDGF-AA, PDGF-BB, PDGF-AB, PDGF-CC, and PDGF-DD).

According to a specific embodiment, PDGF used in the culture medium of some embodiments of the invention is PDGF-BB.

The PDGF used in the culture medium of some embodiments of the invention can be a purified, a synthetic or a recombinantly expressed PDGF protein (e.g., human PDGF subunit B polypeptide GenBank Accession Nos. NP_148937.1 or NP_002599.1; e.g., human PDGF subunit B polynucleotide GenBank Accession Nos. NM_002608 or NM_033016; e.g., human PDGF subunit A polypeptide GenBank Accession Nos. NP_002598.4 or NP_148983.1; e.g., human PDGF subunit A polynucleotide GenBank Accession No. NM_033023). PDGF can be obtained from various commercial sources such as RayBiotech, Abcam Proteins and ProSci Proteins. According to one embodiment, PDGF is obtained from Peprotech (Cat: 100-14B).

According to a specific embodiment, the platelet-derived growth factor comprises the protein encoded by the gene comprising the symbol PDGFB.

It should be noted that for the preparation of a xeno-free culture medium the PDGF is preferably purified from a human source or is recombinantly expressed as is further described hereinbelow.

According to some embodiments of the invention, the concentration of PDGF in the culture medium is in the range of about 0.05 ng/ml to about 1 μg/ml, e.g., from about 0.1 ng/ml to about 1 μg/ml, e.g. from about 0.5 ng/ml to about 100 ng/ml, e.g. from about 1 ng/ml to about 100 ng/ml, e.g. from about 1 ng/ml to about 50 ng/ml, e.g. from about 5 ng/ml to about 50 ng/ml, e.g. from about 5 ng/ml to about 100 ng/ml, e.g. from about 10 ng/ml to about 50 ng/ml.

According to some embodiments of the invention, the concentration of PDGF in the culture medium is at least e.g. about 0.5 ng/ml, e.g. at least about 1 ng/ml, e.g. at least about 2.5 ng/ml, e.g. at least about 5 ng/ml, e.g. at least about 7.5 ng/ml, e.g. at least about 10 ng/ml, e.g. at least about 15 ng/ml, e.g. at least about 20 ng/ml, e.g. at least about 25 ng/ml, e.g. at least about 30 ng/ml, e.g. at least about 40 ng/ml, e.g. at least about 50 ng/ml, e.g. at least about 60 ng/ml, e.g. at least about 70 ng/ml, e.g. at least about 80 ng/ml, e.g. at least about 90 ng/ml, e.g. at least about 100 ng/ml.

According to some embodiments of the invention, the concentration of PDGF in the culture medium is at most e.g. about 1 ng/ml, at most e.g. about 5 ng/ml, at most e.g. about 10 ng/ml, at most e.g. about 15 ng/ml, at most e.g. about 20 ng/ml, at most e.g. about 25 ng/ml, at most e.g. about 50 ng/ml, at most e.g. about 75 ng/ml, at most e.g. about 100 ng/ml.

According to a specific embodiment, the concentration of PDGF (e.g. PDGF-BB) in the culture medium is about 5 ng/ml.

According to a specific embodiment, the concentration of PDGF (e.g. PDGF-BB) in the culture medium is about 10 ng/ml.

According to a specific embodiment, the concentration of PDGF (e.g. PDGF-BB) in the culture medium is about 25 ng/ml.

According to a specific embodiment, the concentration of PDGF (e.g. PDGF-BB) in the culture medium is about 50 ng/ml.

According to one embodiment, the culture medium comprises the basal medium DMEM/F12, GIBCO™ Knockout™ Serum Replacement (KoSR), ITS (Insulin, Transferrin and Selenium) and PDGF (e.g. PDGF-BB). According to a specific embodiment, this culture medium is serum-free. According to a specific embodiment, this culture medium comprises FGF2. According to a specific embodiment, this culture medium comprises TGFβ₁. According to a specific embodiment, this culture medium comprises TGFβ₃.

According to one embodiment, the culture medium comprises the basal medium DMEM/F12, GIBCO™ Knockout™ Serum Replacement (KoSR), ITS (Insulin, Transferrin and Selenium) and PDGF (e.g. PDGF-BB). According to a specific embodiment, this culture medium is serum-free. According to a specific embodiment, this culture medium comprises FGF4. According to a specific embodiment, this culture medium comprises TGFβ₁. According to a specific embodiment, this culture medium comprises TGFβ₃.

According to one embodiment, the culture medium comprises the basal medium DMEM/F12, GIBCO™ Knockout™ Serum Replacement (KoSR), ITS (Insulin, Transferrin and Selenium) and PDGF (e.g. PDGF-BB). According to a specific embodiment, this culture medium is serum-free. According to a specific embodiment, this culture medium comprises FGF2 and FGF4. According to a specific embodiment, this culture medium comprises TGFβ₁. According to a specific embodiment, this culture medium comprises TGFβ₃.

According to some embodiments of the invention the culture medium comprises epidermal growth factor (EGF).

As used herein the term “Epidermal growth factor” or “EGF” refers to any polypeptide of the epidermal growth factor (EGF) family of proteins, or variant thereof, that stimulates cell growth and differentiation. Typically EGF exerts its activity by binding to the epidermal growth factor receptor.

Accordingly, any variant of the EGF molecules that maintains their biological activity; for example, C-terminal truncated molecules (Calnan et al., Gut 2000, 47: 622-627); or molecules truncated at the N-terminal (Svodoca et. al., Biochim. Biophys. Acta 1994, 1206: 35-41; Shin et al., Peptides 1995, 16: 205-210) may be used in line with the present teachings.

The EGF used in the culture medium of some embodiments of the invention can be a purified, a synthetic or a recombinantly expressed EGF protein (e.g., human EGF polypeptide GenBank Accession Nos. NP_001954.2, NP_001171602.1, NP_001343950.1 or NP_001171601.1; e.g., human EGF polynucleotide GenBank Accession Nos. NM_001963 NM_001963). EGF can be obtained from various commercial sources such as RayBiotech, Abcam Proteins and ProSci Proteins. According to one embodiment, EGF is obtained from Peprotech (Cat: AF-100-15).

According to a specific embodiment, the epidermal growth factor comprises the protein encoded by the gene comprising the symbol EGF.

It should be noted that for the preparation of a xeno-free culture medium the EGF is preferably purified from a human source or is recombinantly expressed as is further described hereinbelow.

According to some embodiments of the invention, the concentration of EGF in the culture medium is in the range of about 0.05 ng/ml to about 1 μg/ml, e.g., from about 0.1 ng/ml to about 1 μg/ml, e.g. from about 0.5 ng/ml to about 100 ng/ml, e.g. from about 1 ng/ml to about 100 ng/ml, e.g. from about 1 ng/ml to about 50 ng/ml, e.g. from about 5 ng/ml to about 50 ng/ml, e.g. from about 10 ng/ml to about 50 ng/ml.

According to some embodiments of the invention, the concentration of EGF in the culture medium is at least e.g. about 0.5 ng/ml, e.g. at least about 1 ng/ml, e.g. at least about 2.5 ng/ml, e.g. at least about 5 ng/ml, e.g. at least about 7.5 ng/ml, e.g. at least about 10 ng/ml, e.g. at least about 15 ng/ml, e.g. at least about 20 ng/ml, e.g. at least about 25 ng/ml, e.g. at least about 30 ng/ml, e.g. at least about 40 ng/ml, e.g. at least about 50 ng/ml, e.g. at least about 60 ng/ml, e.g. at least about 70 ng/ml, e.g. at least about 80 ng/ml, e.g. at least about 90 ng/ml, e.g. at least about 100 ng/ml.

According to some embodiments of the invention, the concentration of EGF in the culture medium is at most e.g. about 1 ng/ml, at most e.g. about 5 ng/ml, at most e.g. about 10 ng/ml, at most e.g. about 15 ng/ml, at most e.g. about 20 ng/ml, at most e.g. about 25 ng/ml, at most e.g. about 50 ng/ml, at most e.g. about 75 ng/ml, at most e.g. about 100 ng/ml.

According to a specific embodiment, the concentration of EGF in the culture medium is about 1 ng/ml.

According to a specific embodiment, the concentration of EGF in the culture medium is about 5 ng/ml.

According to a specific embodiment, the concentration of EGF in the culture medium is about 10 ng/ml.

According to a specific embodiment, the concentration of EGF in the culture medium is about 25 ng/ml.

According to a specific embodiment, the concentration of EGF in the culture medium is about 50 ng/ml.

According to one embodiment, the culture medium comprises the basal medium DMEM/F12, GIBCO™ Knockout™ Serum Replacement (KoSR), ITS (Insulin, Transferrin and Selenium) and EGF. According to a specific embodiment, this culture medium is serum-free. According to a specific embodiment, this culture medium comprises FGF2. According to a specific embodiment, this culture medium comprises TGFβ₁. According to a specific embodiment, this culture medium comprises TGFβ₃. According to a specific embodiment, this culture medium comprises PDGF (e.g. PDGF-BB).

According to one embodiment, the culture medium comprises the basal medium DMEM/F12, GIBCO™ Knockout™ Serum Replacement (KoSR), ITS (Insulin, Transferrin and Selenium) and EGF. According to a specific embodiment, this culture medium is serum-free. According to a specific embodiment, this culture medium comprises FGF4. According to a specific embodiment, this culture medium comprises TGFβ₁. According to a specific embodiment, this culture medium comprises TGFβ₃. According to a specific embodiment, this culture medium comprises PDGF (e.g. PDGF-BB).

According to one embodiment, the culture medium comprises the basal medium DMEM/F12, GIBCO™ Knockout™ Serum Replacement (KoSR), ITS (Insulin, Transferrin and Selenium) and EGF. According to a specific embodiment, this culture medium is serum-free. According to a specific embodiment, this culture medium comprises FGF2 and FGF4. According to a specific embodiment, this culture medium comprises TGFβ₁. According to a specific embodiment, this culture medium comprises TGFβ₃. According to a specific embodiment, this culture medium comprises PDGF (e.g. PDGF-BB).

According to some embodiments of the invention, the culture medium further comprises L-glutamine. The concentration of L-glutamine in the culture medium can be from about 0.5 millimolar (mM) to about 10 mM, e.g., about 0.1-5 mM, e.g., about 1-5 mM, e.g., about 2-5 mM, e.g., 1 mM, e.g. 2 mM. L-glutamine can be obtained from various suppliers such as Biological Industries, Beit HaEmek, Israel.

According to some embodiments of the invention, the culture medium further comprises non-essential amino acids (NEAA). Non-essential amino acids can be obtained as a stock of 10 mM from various suppliers such as Invitrogen Corporation products, Grand Island N.Y., USA. The concentration of the non-essential amino acid in the culture medium can be from about 0.1-10%, e.g., about 0.2-5%, e.g., about 0.5-2%, e.g., about 1%, e.g., about 0.5%.

According to some embodiments of the invention, the culture medium further comprises a reducing agent such as beta-mercaptoethanol (0-mercaptoethanol), at a concentration range between about 0.01-1 mM, e.g., 0.05 mM, e.g., 0.1 mM. (β-mercaptoethanol can be obtained from various suppliers such as Gibco.

According to some embodiments of the invention, the culture medium further comprises antibiotics to control the growth of bacterial and fungal contaminants. According to one embodiment, a penicillin-streptomycin solution can be obtained e.g. from Life Technologies or Millipore-Sigma. The concentration of penicillin in the culture medium can be from about 5-100 U/ml, e.g. 10-75 U/ml, e.g. 50 U/ml. The concentration of streptomycin in the culture medium can be from about 0.01-1 mg/ml, e.g. 0.01-0.1 mg/ml, e.g. 0.05 mg/ml.

According to a specific embodiment, the culture medium is MSC-Diff 0-F12 as set forth in Table 1A, herein below.

According to a specific embodiment, the culture medium is MSC-Diff 0-HG as set forth in Table 1B, herein below.

According to a specific embodiment, the culture medium is MSC-Diff 1-15 as set forth in Table 2, herein below.

According to a specific embodiment, the culture medium is MSC-Diff 16-34 or MSC-Diff 40-50 as set forth in Table 3, herein below.

According to a specific embodiment, the culture medium is MSC-Diff 35 as set forth in Table 5A, herein below.

According to a specific embodiment, the culture medium is MSC-Diff 51 as set forth in Table 5B, herein below.

According to a specific embodiment, the culture medium is MSC-Diff 52 as set forth in Table 5C, herein below.

According to a specific embodiment, the culture medium is MSC-Diff 36 as set forth in Table 6, herein below.

According to a specific embodiment, the culture medium is MSC-Diff 37 as set forth in Table 7, herein below.

According to a specific embodiment, the culture medium is MSC-Diff 38 as set forth in Table 8, herein below.

According to a specific embodiment, the culture medium is MSC-Diff 39 as set forth in Table 9, herein below.

According to a specific embodiment, the culture medium is as set forth in Table 11A, below.

According to a specific embodiment, the culture medium as set forth in Table 11A further comprises at least one component of the components set forth in Table 11B.

According to a specific embodiment, the culture medium is as set forth in Tables 12A-1B, below.

TABLE 11A
Culture medium

| Reagent name | Final Concentration range | |
|---|---|---|
| DMEM/F12/ | 50-90% | |
| DMEM/ | | |
| F12/ | | |
| MEM Alpha medium/ | | |
| DMEM HG | | |
| KoSR | 5-15% | |
| ITS | 0.25-3% | |
| NEAA | 0.5-3% | |
| β-mercaptoethanol | 0.05-0.5 | mM |
| L-glutamine | 1-5 | mM |

| | |
|---|---|
| Penicillin-Streptomycin Solution | Penicillin: 50-200 U/ml |
| | Streptomycin: 0.05-3 mg/ml |

TABLE 11B
Culture medium components

| | | |
|---|---|---|
| Glucose | 1-5 | gr/Liter |
| L-ascorbic acid | 20-200 | μg/ml |
| Sodium pyruvate | 20-200 | μg/ml |

TABLE 12A
Culture medium

| Reagent name | Final Concentration |
|---|---|
| DMEM/F12/DMEM/F12 | 30-60% |
| MEM Alpha medium | 30-60% |
| FBS | 5-30% |
| KoSR | 3-10% |
| ITS | 0.1-0.8% |
| bFGF stock solution | (optional component-20-60 ng/ml) |
| NEAA | 0.5-5% |
| β-mercaptoethanol | 0.05-0.4% |
| L-glutamine | 1-5 mM |
| Penicillin-Streptomycin | Penicillin: 50-200 U/ml |
| Solution | Streptomycin: 0.05-3 mg/ml |

TABLE 12B
Culture medium

| Reagent name | Final Concentration |
|---|---|
| DMEM/F12/DMEM/ | 70-95% |
| F12/MEM Alpha medium | |
| FBS or KoSR | 5-30% |
| ITS | 0.1-2% |
| bFGF stock solution | (optional component-20-60 ng/ml) |
| NEAA | 0.5-5% |
| β-mercaptoethanol | (optional component-0.05-0.4%) |
| L-glutamine | 1-5 mM |
| Penicillin-Streptomycin | Penicillin: 50-200 U/ml |
| Solution | Streptomycin: 0.05-3 mg/ml |

According to a specific embodiment, the culture medium is a defined culture medium comprising a basal medium comprising DMEM/F12, knockout serum replacement (KoSR) at a concentration of at least about 5%, and ITS (Insulin, Transferrin and Selenium) at a concentration of at least about 0.5%, wherein the culture medium is serum-free, and further wherein the culture medium is capable of promoting differentiation of pluripotent stem cells (PSCs) into mesenchymal stem cells (MSCs) or supporting expansion of MSCs. According to a specific embodiment, this culture medium further comprises bFGF (e.g. FGF2) at a concentration of at least about 1 ng/ml.

According to a specific embodiment, the culture medium is a defined culture medium comprising a basal medium comprising DMEM/F12, knockout serum replacement (KoSR) at a concentration of at least 5%, ITS (Insulin, Transferrin and Selenium) at a concentration of at least 0.5% and L-ascorbic acid at a concentration of at least 25 μg/ml, wherein the culture medium is serum-free, and further wherein the culture medium is capable of promoting differentiation of pluripotent stem cells (PSCs) into mesenchymal stem cells (MSCs) or supporting expansion of MSCs.

According to a specific embodiment, the culture medium is a defined culture medium comprising a basal medium comprising DMEM/F12, knockout serum replacement (KoSR) at a concentration of at least 5%, ITS (Insulin, Transferrin and Selenium) at a concentration of at least 0.5%, L-ascorbic acid at a concentration of at least 25 μg/ml and Sodium pyruvate at a concentration of at least 25 μg/ml, wherein the culture medium is serum-free, and further wherein the culture medium is capable of promoting differentiation of pluripotent stem cells (PSCs) into mesenchymal stem cells (MSCs) or supporting expansion of MSCs.

According to a specific embodiment, the culture medium is a defined culture medium comprising a basal medium comprising DMEM/F12, knockout serum replacement (KoSR) at a concentration of at least 5%, ITS (Insulin, Transferrin and Selenium) at a concentration of at least 0.5%, glucose at a concentration of at least 4 gr/Liter, L-ascorbic acid at a concentration of at least 25 μg/ml and Sodium pyruvate at a concentration of at least 25 μg/ml, wherein the culture medium is serum-free, and further wherein the culture medium is capable of promoting differentiation of pluripotent stem cells (PSCs) into mesenchymal stem cells (MSCs) or supporting expansion of MSCs.

According to a specific embodiment, the culture medium is a defined culture medium comprising a basal medium comprising DMEM HG, knockout serum replacement (KoSR) at a concentration of at least 5%, ITS (Insulin, Transferrin and Selenium) at a concentration of at least 0.5%, L-ascorbic acid at a concentration of at least 25 μg/ml and Sodium pyruvate at a concentration of at least 25 μg/ml, wherein the culture medium is serum-free, and further wherein the culture medium is capable of promoting differentiation of pluripotent stem cells (PSCs) into mesenchymal stem cells (MSCs) or supporting expansion of MSCs.

According to a specific embodiment, the culture medium comprises a basal medium comprising DMEM/F12 and MEM alpha at a volume ratio ranging between 0.6 to 1.5 DMEM/F12 to MEM alpha, a knockout serum replacement (KoSR) at a concentration of at least about 3%, ITS (Insulin, Transferrin and Selenium) at a concentration of at least about 0.1%, and serum at a concentration of at least about 5%, and further wherein the culture medium is capable of promoting differentiation of pluripotent stem cells (PSCs) into mesenchymal stem cells (MSCs) or supporting expansion of MSCs.

According to a specific embodiment, the culture medium comprises a basal medium comprising MEM alpha ITS (Insulin, Transferrin and Selenium) at a concentration of at least about 0.1%, and serum at a concentration of at least about 5%, and further wherein the culture medium is capable of promoting differentiation of pluripotent stem cells (PSCs) into mesenchymal stem cells (MSCs).

According to a specific embodiment, the culture medium comprises a basal medium comprising MEM alpha ITS (Insulin, Transferrin and Selenium) at a concentration of at least about 0.1%, and serum at a concentration of at least about 20%, and further wherein the culture medium is capable of promoting differentiation of pluripotent stem cells (PSCs) into mesenchymal stem cells (MSCs).

As mentioned, any of the proteinaceous factors used in the culture medium of the present invention [e.g., bFGF (e.g. FGF2, FGF4), EGF, PDGF (e.g. PDGF-BB), TGFβ1, TGFβ₃, WNT-3A, insulin, selenium, albumin, transferrin) can be recombinantly expressed or biochemically synthesized. In addition, naturally occurring proteinaceous factors (such as bFGF, EGF, PDGF, WNT-3A and TGFβ) can be purified from biological samples (e.g., from human serum, cell cultures) using methods well known in the art. It should be noted that for the preparation of an animal contaminant-free culture medium the proteinaceous factor is preferably purified from a human source or is recombinantly expressed.

Biochemical synthesis of the proteinaceous factors of the present invention can be performed using standard solid phase techniques. These methods include exclusive solid phase synthesis, partial solid phase synthesis methods, fragment condensation and classical solution synthesis.

Recombinant expression of the proteinaceous factors of the present invention can be generated using recombinant techniques such as described by Bitter et al., (1987) Methods in Enzymol. 153:516-544; Studier et al. (1990) Methods in Enzymol. 185:60-89; Brisson et al. (1984) Nature 310:511-514; Takamatsu et al. (1987) EMBO J. 6:307-311; Coruzzi et al. (1984) EMBO J. 3:1671-1680; Brogli et al., (1984) Science 224:838-843; Gurley et al. (1986) Mol. Cell. Biol. 6:559-565; and Weissbach & Weissbach, 1988, Methods for Plant Molecular Biology, Academic Press, NY, Section VIII, pp 421-463, which are fully incorporated herein by reference.

As mentioned, the culture medium of some embodiments of the invention is capable of supporting expansion of MSCs.

According to one aspect there is provided a method of expanding mesenchymal stem cells (MSCs) without differentiation, the method comprising culturing the MSCs in the culture medium of some embodiments of the invention, thereby culturing the MSCs under culturing conditions which allow expansion of the MSCs without differentiation (i.e. into cells of a specialized type such as e.g. adipocytes, osteocytes, and chondrocytes).

As used herein the term “expansion” or “expanding” refers to increasing the number of MSCs over the culturing period (by at least about 5%, 10%, 15%, 20%, 30%, 40%, 50%, 60%, 70%, 80%, 90%, 100% or more, e.g. by 2, 3, 4, 5, 6, 7, 8, 9, 10 fold or more). It will be appreciated that the number of MSCs, which can be obtained from a single MSC, depends on the proliferation capacity of the MSC. The proliferation capacity of a MSC can be calculated by the doubling time of the cell (i.e., the time needed for a cell to undergo a mitotic division in the culture) and the period the mesenchymal stem cell culture can be maintained in the undifferentiated state (which is equivalent to the number of passages multiplied by the days between each passage).

According to some embodiments, the method of the invention enables the expansion of a single MSC (e.g., BM-derived MSC, UC-derived MSC, PSC-derived MSC) by at least about 5-50 fold (e.g. 15-20 fold) in 7-15 days in a 2D culture (as further discussed below).

According to some embodiments, the method of the invention enables the expansion of a single MSC (e.g., BM-derived MSC, UC-derived MSC, PSC-derived MSC) by at least about 100-1000 fold (e.g. 500-700 fold) in 15-25 days (e.g. in 20 days) in a 3D culture (as further discussed below).

For example, as described in the Examples section which follows, the MSCs can be maintained in the proliferative, multipotent and undifferentiated state in the presence of the MSC culture media (e.g. in each of MSC-Diff 0-35 or MSC-Diff 40-42 media described in Tables 1-3, below) for at least 5-25 passages when cultured in 2D adherent cultures (with or without coating) or in a suspension (3D) culture. Given that each passage occurs every 3-7 days, the MSCs can be maintained for about 125-175 days (i.e., about 3000-4200 hours). Given that the MSC doubling time was 25-250 hours, a single MSC cultured under these conditions could be expanded to give rise to 6×10⁷-1.2×10⁸ MSCs.

According to one embodiment, MSCs can be cultured in a 2-dimensional (2D) culture.

The term “2-dimensional culture” or “2D culture” refers to the growth of cells on a flat dish, typically made of plastic (e.g. polystyrene cell culture plate).

The 2D cultures may comprise coated surfaces onto which the cells can adhere and spread. Exemplary materials for coating 2D culture surfaces suitable for MSCs expansion include, but are not limited to, extracellular matrix (ECM) proteins or other commercially available cell adhesion factors such as adhesive proteins, e.g. fibronectin (FN) or vitronectin (VN), and other coating proteins, e.g. laminin (LN), collagen type I (COL I), collagen type IV (COL IV), and gelatin. Additional coating strategies for 2D cultures of MSCs are discussed in Cimino et al., Stem Cells International (2017) Article ID 6597815, incorporated herein by reference. According to one embodiment, the 2D cultures are not coated.

According to one embodiment, culturing MSCs in 2D cultures is affected by planting the MSCs in a culture plate at a cell density which promotes cell survival and proliferation but limits differentiation. Typically, a plating density (or a seeding density) of between about 4×10³ cells/cm² to about 2×10⁴ cells/cm² is used.

In order to provide the MSCs with sufficient and constant supply of nutrients and growth factors while in the 2D culture, the culture medium can be replaced on a daily basis, or, at a pre-determined schedule such as every 2-7 days (e.g. 2-3 days). For example, replacement of the culture medium when the cells are grown in 2D culture and adhere to the plate can be performed by aspirating the medium from the culture dish and addition of fresh medium.

According to one embodiment, MSCs can be cultured in a three-dimensional (3D) culture.

The term “3-dimensional culture” or “3D culture” refers to a cell culture with cells positioned relative to each other in three dimensions, i.e. width, depth and height.

According to one embodiment, the 3D culture is a suspension culture.

As used herein the phrase “suspension culture” refers to a culture in which the cells are suspended in a medium rather than adhering to a surface.

According to some embodiments of the invention, the conditions for culturing the mesenchymal stem cells in suspension are devoid of substrate adherence, e.g., without adherence to an external substrate such as components of extracellular matrix, a glass microcarrier or beads (e.g. carrier-free suspension culture).

According to some embodiments of the invention, at least some of the MSCs (e.g. adult MSCs) in the suspension culture adhere to the vessel surface.

Culturing MSCs in a suspension culture according to the method of some embodiments of the invention is affected by plating the MSCs in a culture vessel at a cell density which promotes cell survival and proliferation but limits differentiation. Typically, a plating density (or a seeding density) of between about 1×10⁵ cells/ml to about 3×10⁶ cells/ml is used. Similar concentrations are used when a bioreactor is used (as discussed below).

In order to provide the MSCs with sufficient and constant supply of nutrients and growth factors while in the suspension culture, the culture medium can be replaced on a daily basis, or, at a pre-determined schedule such as every 2-7 days (e.g. 2-3 days). For example, replacement of the culture medium can be performed by subjecting the MSC suspension culture to centrifugation for about 3 minutes at 300-400 g and resuspension of the formed MSC pellet in a fresh medium. Additionally or alternatively, a culture system in which the culture medium is subject to constant filtration or dialysis so as to provide a constant supply of nutrients or growth factors to the MSCs may be employed.

The culture vessel used for culturing the MSCs in suspension according to the method of some embodiments of the invention can be any tissue culture vessel (e.g., with a purity grade suitable for culturing MSCs). Preferably, in order to obtain a scalable culture, culturing according to some embodiments of the invention is affected using a controlled culturing system (preferably a computer-controlled culturing system) in which culture parameters such as temperature, agitation, pH, and pO₂ is automatically performed using a suitable device. Once the culture parameters are recorded, the system is set for automatic adjustment of culture parameters as needed for pluripotent stem cells expansion.

According to some embodiments of the invention, culturing is affected under conditions comprising a dynamic suspension culture.

The phrase “dynamic suspension culture” refers to conditions in which the cells are subject to constant movement while in the suspension culture. Such conditions are further discussed herein below.

According to one embodiment, the dynamic suspension culture utilizes a Wave reactor, a stirred reactor or a spinner flask (e.g. glass spinner flask).

According to some embodiments of the invention, culturing is affected under conditions comprising a static (i.e., non-dynamic) suspension culture.

The phrase “static suspension culture” refers to conditions in which the cells are subject to stationary conditions while in the suspension culture. Such conditions are further discussed herein below.

According to one embodiment, the static suspension culture utilizes a flask (e.g. Erlenmeyer flask) or a Petri dish (available from e.g. Greiner, Frickenhausen, Germany).

As used herein the term “passage” or “passaging” as used herein refers to splitting the cells in the culture vessel to 2 or more culture vessels, typically including addition of fresh culture medium. Passaging is typically done when the cells reach a certain density in culture.

According to some embodiments of the invention, passaging of a MSC culture seeded at a concentration of about 1×10⁶ cells per milliliter under 2D culture system is done when the cells' concentration increases to about 2 or 3 folds (e.g., at a concentration of about 2×10⁶-3×10⁶ cells/ml), but no more than up to about 4 folds (e.g., at a concentration about 4×10⁶ cells/ml).

According to some embodiments of the invention, passaging of a MSC culture seeded at a concentration of about 1×10⁶ cells per milliliter under static 3D culture system (i.e. non-dynamic) is done when the cells' concentration increases to about 2 or 3 folds (e.g., at a concentration of about 2×10⁶-3×10⁶ cells/ml), but no more than up to about 4 folds (e.g., at a concentration about 4×10⁶ cells/ml).

According to some embodiments of the invention, passaging of a MSC culture seeded at a concentration of about 1×10⁶ cells per milliliter under dynamic 3D culture system (e.g. in a spinner flask) is done when the cells' concentration increases about 20-40 folds (e.g., at a concentration of about 20×10⁶-40×10⁶ cells/ml), but no more than up to about 50 folds (e.g., at a concentration of about 50×10⁶ cells/ml).

According to some embodiments of the invention the culture medium is capable of maintaining MSCs in a proliferative, multipotent and undifferentiated state for at least about 2 passages, at least about 5 passages, at least about 10 passages, at least about 15 passages, at least about 20 passages, at least about 25 passages, at least about 30 passages, at least about 35 passages, at least about 40 passages, at least about 45 passages, at least about 50 passages and more.

According to a specific embodiment, the culture medium is capable of maintaining MSCs in a proliferative, multipotent and undifferentiated state for 2-50 passages, e.g. for 5-40 passages, e.g. for 5-30 passages, e.g. for 5-25 passages, e.g. for 5-20 passages, e.g. for 5-15 passages, e.g. for 5-10 passages, e.g. for 10-30 passages, e.g. for 10-20 passages, e.g. for 10-15 passages, e.g. for 15-30 passages, e.g. for 15-25 passages, e.g. for 15-20 passages, e.g. for 20-40 passages, e.g. for 20-30 passages, e.g. for 20-25 passages, e.g. for 30-40 passages, e.g. for 40-50 passages.

According to some embodiments of the invention, the MSCs which are included in the cell culture of some embodiments of the invention exhibit a stable karyotype (chromosomal stability) during the culturing period, e.g., for at least 2 passages, e.g., at least 4 passages, e.g., at least 8 passages, e.g., at least 15 passages, e.g., at least 20 passages, e.g., at least 25 passages, e.g., at least 30 passages, e.g., at least 35 passages, e.g., at least 40 passages, e.g., at least 45 passages, e.g., at least 50 passages.

According to some embodiments of the invention, MSCs are cultured for no more than about 10 passages, about 15 passages, about 20 passages, about 25 passages, about 30 passages or about 35 passages.

According to some embodiments of the invention, the cell culture of the invention maintains the MSCs in a non-tumorigenic, genetically stable state.

According to some embodiments of the invention, the cell culture of the invention is characterized by at least 30%, at least 40%, at least 50%, at least 60%, e.g., at least 70%, e.g., at least 80%, e.g., at least 85%, e.g., at least 90%, e.g., at least 95% of undifferentiated multipotent mesenchymal stem cells.

According to some embodiments of the invention, the MSCs expanded according to the described methods are capable of differentiating into any one of an adipogenic lineage, an osteoblastic lineage, and a chondrogenic lineage.

As mentioned, the culture medium of some embodiments of the invention is capable of promoting differentiation of PSCs into MSCs.

The term “differentiation” or “differentiating” is a relative term that refers to a developmental process by which a cell (e.g. pluripotent stem cell) has progressed further down a developmental pathway. Thus in some embodiments, a pluripotent stem cell (PSC) can differentiate into a multipotent mesenchymal stem cell.

According to one aspect, there is provided a method of generating mesenchymal stem cells (MSCs) from pluripotent stem cells (PSCs), the method comprising culturing the PSCs in a suspension culture in the culture medium of some embodiments of the invention under culturing conditions suitable for differentiation of the PSCs to MSCs, thereby generating the MSCs.

According to one aspect, there is provided a method of generating mesenchymal stem cells (MSCs) from pluripotent stem cells (PSCs), the method comprising culturing the PSCs in a suspension culture in a culture medium comprising a basal medium comprising MEM alpha, serum and ITS (Insulin, Transferrin and Selenium), under culturing conditions suitable for differentiation of the PSCs to MSCs, thereby generating the MSCs.

According to one aspect, there is provided a method of generating mesenchymal stem cells (MSCs) from pluripotent stem cells (PSCs), the method comprising culturing the PSCs in a suspension culture in a culture medium comprising a basal medium comprising MEM alpha, serum at a concentration of at least 5%, and ITS (Insulin, Transferrin and Selenium) at a concentration of at least 1%, under culturing conditions suitable for differentiation of the PSCs to the MSCs, thereby generating the MSCs.

According to one embodiment, culturing of PSCs is affected in a suspension culture (i.e. 3D culture, as discussed above).

According to some embodiments of the invention, the conditions for culturing the pluripotent stem cells in suspension are devoid of substrate adherence, e.g., without adherence to an external substrate such as components of extracellular matrix, a glass microcarrier or beads.

It should be noted that some protocols of culturing pluripotent stem cells such as hESCs and iPS cells include microencapsulation of the cells inside a semipermeable hydrogel membrane, which allows the exchange of nutrients, gases, and metabolic products with the bulk medium surrounding the capsule (for details see e.g., U.S. Patent Application No. 20090029462 to Beardsley et al.).

According to some embodiments of the invention, the pluripotent stem cells cultured in the suspension culture are devoid of cell encapsulation.

According to some embodiments of the invention, the culture medium and/or the conditions for culturing the pluripotent stem cells in suspension are devoid of a protein carrier, i.e. a protein which acts in the transfer of proteins or nutrients (e.g., minerals such as zinc) to the cells in the culture. Such protein carriers can be, for example, albumin (e.g., bovine serum albumin), Albumax (lipid enriched albumin) or plasmanate (human plasma isolated proteins). Since these carriers are derived from either human or animal sources their use in hESCs of human iPS cell cultures is limited by batch-specific variations and/or exposure to pathogens. Thus, a culture medium which is devoid of a protein carrier (e.g., albumin) is highly advantageous since it enables a truly defined medium that can be manufacture from recombinant or synthetic materials.

Culturing PSCs in a suspension culture according to the method of some embodiments of the invention is affected by plating the pluripotent stem cells in a culture vessel at a cell density which promotes cell differentiation. Typically, a plating density (or a seeding density) of between about 1×10⁵ cells/ml to about 3×10⁶ cells/ml is used. Similar concentrations are used when a bioreactor is used. It will be appreciated that although single-cell suspensions of stem cells are usually seeded, small clusters such as of 10-200 cells may also be used.

In order to provide the pluripotent stem cells with sufficient and constant supply of nutrients and growth factors while in the suspension culture, the culture medium can be replaced on a daily basis, or, at a pre-determined schedule such as every 2-3 days. For example, replacement of the culture medium can be performed by subjecting the pluripotent stem cells suspension culture to centrifugation for about 3 minutes at 300-400 g, and resuspension of the formed pluripotent stem cells pellet in a fresh medium. Additionally, or alternatively, a culture system in which the culture medium is subject to constant filtration or dialysis so as to provide a constant supply of nutrients or growth factors to the pluripotent stem cells may be employed.

The culture vessel used for culturing the pluripotent stem cells in suspension according to the method of some embodiments of the invention can be any tissue culture vessel (e.g., with a purity grade suitable for culturing pluripotent stem cells) having an internal surface designed such that pluripotent stem cells cultured therein are unable to adhere or attach to such a surface (e.g., non-tissue culture treated, to prevent attachment or adherence of cells to the surface). Preferably, in order to obtain a scalable culture, culturing according to some embodiments of the invention is affected using a controlled culturing system (preferably a computer-controlled culturing system) in which culture parameters such as temperature, agitation, pH, and pO₂ is automatically performed using a suitable device. Once the culture parameters are recorded, the system is set for automatic adjustment of culture parameters as needed for pluripotent stem cells expansion.

According to some embodiments of the invention, culturing is affected under conditions comprising a static (i.e., non-dynamic) suspension culture.

For non-dynamic culturing of pluripotent stem cells, the pluripotent stem cells can be cultured in uncoated 58 mm Petri dishes (Greiner, Frickenhausen, Germany). For example, to initiate a suspension culture on 58 mm Petri dishes the pluripotent stem cells are seeded at a cell density of 1×10⁶-5×10⁶ cells/dish.

According to some embodiments of the invention, culturing is affected under conditions comprising a dynamic suspension culture (e.g., using a Wave reactor or stirred reactor).

For dynamic culturing of pluripotent stem cells, the pluripotent stem cells can be cultured in spinner flasks [e.g., of 200 ml to 1000 ml, for example 250 ml which can be obtained from CellSpin of Integra Biosciences, Fernwald, Germany; of 100 ml which can be obtained from Bellco, Vineland, N.J.; or in 125 ml Erlenmeyer (Corning Incorporated, Corning N.Y., USA)] which can be connected to a control unit and thus present a controlled culturing system. The culture vessel (e.g., a spinner flask, an Erlenmeyer) is shaken continuously. According to some embodiments of the invention the culture vessels are shaken at 40-110 rounds per minute (rpm) using magnetic plate and placed in the incubator. Additionally, or alternatively, the culture vessel can be shaken using a shaker (S3.02.10L, ELMI ltd, Riga, Latvia). According to some embodiments of the invention the culture medium is changed every 1-3 days, e.g., every day. Other suitable controlled-bioreactors which stir the medium by an impeller and can be used for dynamic culturing of the pluripotent stem cells in the culture medium according to some embodiments of the invention include the Biostat® Aplus cell culture (Sartorius North America, Edgewood, N.Y., USA), Cell Optimizer controlled bioreactor (Wheaton Science Products, Millville, N.J., USA) equipped with Cell Lift impeller (Infors HT, Rittergasse, Switzerland), Informs HT Multifors stirred reactor (Informs GA, CH-4103 Bottmingen Switzerland).

Additionally or alternatively, dynamic culturing of pluripotent stem cells can be achieved using a controlled bioreactor in which the dynamics of the cells is achieved by a wave-like motion, such as the Biostat® Cultibag RM (Sartorius North America, Edgewood, N.Y., USA) (2 litter bag with 1 litter). The reactor parameters may include a speed of tilting: 10-16 rounds per minute (rpm); angle 7°; Temperature: 37° C., PH: 7-7.4, O₂ concentration: 50%. Another suitable bioreactor is the WavePod system 20/50 EH5 Wave Bioreactor (GE Healthcare, USA), which while using the same parameters enables increase in 70 folds during 12 days. Additional suitable bioreactor is the 55 ml RWV/STLV bioreactor which allows minimum shear forces within the reactor (Synthecon Incorporated, Houston, Tex., USA).

For example, to initiate a suspension culture under dynamic conditions, the pluripotent stem cells are seeded at a concentration of about 10⁴-10⁶ cells/ml.

While in the dynamic suspension culture, the pluripotent stem cells can be passaged every 5-7 days by dissociating the cell clumps (as described below). Since the bioreactors have a large capacity, the cell culture needs no further splitting into additional culture vessels and only addition and/or replacement of medium with a fresh medium can be performed every 3-10 days.

According to some embodiments of the invention, the suspension culture of PSCs comprises clumps.

As used herein the term “clump” refers to a cluster of cells which adhere to each other in suspension.

According to some embodiments of the invention, the cell clump remains intact when the medium of the suspension culture is changed (e.g., increased, decreased or replaced) without employing any mechanical or enzymatic dissociation of the clumps.

According to some embodiments of the invention, each of the pluripotent stem cell clumps comprises at least about 250 cells (e.g., about 250), e.g., at least about 500 cells (e.g., about 500), at least about 600 cells (e.g., about 600), at least about 700 cells (e.g., about 700), at least about 800 cells (e.g., about 800), at least about 900 cells (e.g., about 900), at least about 1000 cells (e.g., about 1000), at least about 1100 cells (e.g., about 1100), at least about 1200 cells (e.g., about 1200), at least about 1300 cells (e.g., about 1300), at least about 1400 cells (e.g., about 1400), at least about 1500 cells (e.g., about 1500), at least about 5×10³ cells (e.g., about 5×10³), at least about 1×10⁴ cells (e.g., about 1×10⁴), at least about 5×10⁴ cells (e.g., about 5×10⁴), at least about 1×10⁵ cells (e.g., about 1×10⁵), or more.

According to some embodiments of the invention, passaging of a cell culture seeded at a concentration of about 1×10⁶ cells per milliliter under static 3D culture system (i.e. non-dynamic) is done when the cells' concentration increases to about 2 or 3 folds (e.g., at a concentration of about 2×10⁶-3×10⁶ cells/ml), but no more than up to about 4 folds (e.g., at a concentration about 4×10⁶ cells/ml).

According to some embodiments of the invention, passaging of a cell culture seeded at a concentration of about 1×10⁶ cells per milliliter under dynamic 3D culture system (e.g. in a spinner flask) is done when the cells' concentration increases about 20-40 folds (e.g., at a concentration of about 20×10⁶-40×10⁶ cells/ml), but no more than up to about 50 folds (e.g., at a concentration of about 50×10⁶ cells/ml).

According to some embodiments of the invention, the passaging does not necessarily require dissociation of the cell clumps in the cell culture.

According to some embodiments of the invention, the suspension culture is devoid of clumps.

According to some embodiments of the invention, the suspension culture comprises single cells.

As used herein the phrase “single cells” refers to the state in which the pluripotent stem cells do not form cell clusters (i.e. each cluster comprising more than about 250 pluripotent stem cells) in the suspension culture.

According to some embodiments of the invention, the pluripotent stem cells do not form cell clusters, each cluster of single cells comprising no more than about 200, about 150, about 100, about 90, about 80, about 70, about 60, about 50, about 40, about 30, about 20, about 19, about 18, about 17, about 16, about 15, about 14, about 13, about 12, about 11, about 10, about 9, about 8, about 7, about 6, about 5, about 3, about 2, or about 1 pluripotent stem cell, in the suspension culture.

According to some embodiments of the invention, each of the plurality of the pluripotent stem cells does not adhere to another pluripotent stem cell while in the suspension culture.

According to one embodiment, separating the pluripotent stem cell clumps to single cells is employed by mechanical dissociation, i.e. by employing a physical force rather than an enzymatic activity.

For mechanical dissociation, a pellet of pluripotent stem cells (which may be achieved by centrifugation of the cells) or an isolated pluripotent stem cells clump can be dissociated by pipetting the cells up and down in a small amount of medium (e.g., 0.2-1 ml). For example, pipetting can be performed for several times (e.g., between 3-20 times) using a tip of a 200 μl or 1000 μl pipette.

Additionally or alternatively, mechanical dissociation of large pluripotent stem cells clumps can be performed using a device designed to break the clumps to a predetermined size. Such a device can be obtained from CellArtis Goteborg, Sweden. Additionally or alternatively, mechanical dissociation can be manually performed using a needle such as a 27 g needle (BD Microlance, Drogheda, Ireland) while viewing the clumps under an inverted microscope.

According to one embodiment, separating the pluripotent stem cell clumps to single cells is employed by enzymatic dissociation (e.g. by trypsin, elastase and/or collagenase).

According to some embodiments of the invention, passaging is affected under conditions devoid of enzymatic dissociation.

According to some embodiments of the invention, culturing in suspension is affected under conditions devoid of enzymatic dissociation of cell clusters/clumps.

According to some embodiments of the invention, the culturing conditions are devoid of using an anti-apoptotic agent.

According to some embodiments of the invention, the culturing conditions are affected with an anti-apoptotic agent, e.g. Rho-associated kinase (ROCK) inhibitor. ROCK inhibitors are commercially available from e.g. EMD Biosciences, Inc. La Jolla, Calif., USA.

According to one embodiment, when the pluripotent stem cells in a suspension culture are mechanically passaged without enzymatic dissociation of cell clusters for at least about 2 and no more than about 10 passages, the pluripotent stem cells adopt the single cell mode of cell growth (i.e., they are expanded as single cells and not as cell clumps) and grow without the need of further dissociation of cell clusters for at least about 15, 20 or 25 additional passages (as previously discussed in PCT publication no. WO/2012/032521).

According to some embodiments of the invention, culturing is affected for at least one passage, at least 2, 3, 4, 5, 6, 7, 8, 9, 10, 11, 12, 13, 14, 15, 16, 17, 18, 19, 20 passages in an undifferentiated pluripotent state.

It should be noted that while the cells are cultured as single cells, they still need to be diluted when the concentration of cells exceeds about 1×10⁶ cells per milliliter (e.g., 5×10⁶ cells per 5 ml of Petri dish).

According to one embodiment, differentiation of pluripotent stem cells to mesenchymal stem cells is affected for 3-30 days, e.g. about 3-21 days, e.g. about 3-18 days, e.g. about 3-14 days, e.g. about 3-12 days, e.g. about 3-10 days, e.g. about 3-7 days, e.g. about 3-5 days, e.g. about 5-21 days, e.g. about 5-14 days, e.g. about 5-10 days, e.g. about 5-7 days, e.g. about 7-21 days, e.g. about 7-14 days, e.g. about 7-12 days, e.g. about 7-10 days, e.g. about 10-21 days, e.g. about 10-14 days.

According to one embodiment, differentiation of pluripotent stem cells to mesenchymal stem cells is affected for about 3 days, about 5 days, about 7 days, about 10 days, about 12 days, about 14 days, about 17 days, about 19 days, about 21 days, about 24 days, about 27 days, about 30 days.

According to one embodiment, differentiation of pluripotent stem cells to mesenchymal stem cells is affected for up to about 5 days, about 7 days, about 10 days, about 12 days, about 14 days, about 17 days, about 19 days, about 21 days, about 24 days, about 27 days, about 30 days.

According to a specific embodiment, differentiation of pluripotent stem cells to mesenchymal stem cells is affected for 3-14 days (e.g. about 3, 4, 5, 6, 7, 8, 9, 10, 12, 14 days).

According to a specific embodiment, differentiation of pluripotent stem cells to mesenchymal stem cells is affected for 3-10 days (e.g. about 3, 4, 5, 6, 7, 8, 9, 10 days).

According to a specific embodiment, differentiation of pluripotent stem cells to mesenchymal stem cells is affected for 3-5 days (e.g. about 3, 4, 5 days).

Determination that the pluripotent stem cells have undergone differentiation into mesenchymal stem cells can be carried out using any method known in the art. For example, by determination of expression of cell surface markers (as discussed below), using e.g. FACS analysis. Additionally or alternatively, functional assays can be carried out both in vitro and in vivo, particularly assays relating to the ability of stem cells to give rise to multiple differentiated progeny, assays for responsiveness to canonical WNT signaling, and the like.

According to one embodiment, MSCs are characterized by cell-surface expression of e.g.

CD54, CD9, CD29, CD44, CD56, CD61, CD63, CD71, CD73, CD90, CD97, CD98, CD99, CD105, CD106, CD112, CD146, CD155, CD166, CD276 and/or CD304. According to one embodiment, MSCs are characterized by lack of cell-surface expression of e.g. CD31, CD34, CD20, CD19, CD14 and/or CD45. According to one embodiment, human MSCs may be characterized by the absence of the cell-surface expression of CD14 and/or CD11b; CD19 and/or CD79a; or HLA-DR. It will be appreciated that all of the markers may be expressed on a single MSC cell, or alternatively, the markers may be expressed on different MSC cells (e.g. in the same culture).

According to a specific embodiment, human MSCs are characterized by the expression signature CD105⁺/CD146⁺/CD90⁺/CD44⁺/CD31⁻/CD34⁻/CD45⁻ cells).

According to one embodiment, at least about 20-100%, e.g. about 20-30%, e.g. about 20-40%, e.g. about 20-60%, e.g. about 30-50%, e.g. about 30-70%, e.g. about 40-50%, e.g. about 40-80%, e.g. about 50-60%, e.g. about 50-70%, e.g. about 60-80%, e.g. about 60-90%, e.g. about 70-90%, e.g. about 80-100% of the MSCs generated by the method of some embodiments of the invention are characterized by a CD105⁺/CD146⁺/CD90⁺/CD44⁺/CD31⁻/CD34⁻/CD45⁻ expression signature.

According to some embodiments of the invention, at least about 30% (e.g., 30%), at least about 35% (e.g., 35%), at least about 40% (e.g., 40%), at least about 45% (e.g., 45%), at least about 50% (e.g., 50%), at least about 55% (e.g., 55%), at least about 60% (e.g., 60%), at least about 65% (e.g., 65%), at least about 70% (e.g., 70%), at least about 75% (e.g., 75%), at least about 80% (e.g., 80%), at least about 81% (e.g., 81%), at least about 82% (e.g., 82%), at least about 83% (e.g., 83%), at least about 84% (e.g., 84%), at least about 85% (e.g., 85%), at least about 86% (e.g., 86%), at least about 87% (e.g., 87%), at least about 88% (e.g., 88%), at least about 89% (e.g., 89%), at least about 90% (e.g., 90%), at least about 91% (e.g., 91%), at least about 92% (e.g., 92%), at least about 93% (e.g., 93%), at least about 94% (e.g., 94%), at least about 95% (e.g., 95%), at least about 96% (e.g., 96%), at least about 97% (e.g., 97%), at least about 98% (e.g., 98%), at least about 99% (e.g., 99%), e.g., 100% of the MSCs generated by the method of some embodiments of the invention are characterized by a CD105⁺/CD146⁺/CD90⁺/CD44⁺/CD31⁻/CD34⁻/CD45⁻ expression signature.

According to a specific embodiment, at least 30% of the MSCs generated by the method of some embodiments of the invention are characterized by a CD105⁺/CD146⁺/CD90⁺/CD44⁺/CD31⁻/CD34⁻/CD45⁻ expression signature.

According to a specific embodiment, at least 40% of the MSCs generated by the method of some embodiments of the invention are characterized by a CD105⁺/CD146⁺/CD90⁺/CD44⁺/CD31⁻/CD34⁻/CD45⁻ expression signature.

According to a specific embodiment, at least 50% of the MSCs generated by the method of some embodiments of the invention are characterized by a CD105⁺/CD146⁺/CD90⁺/CD44⁺/CD31⁻/CD34⁻/CD45⁻ expression signature.

According to one embodiment, there is provided an isolated population of mesenchymal stem cells (MSCs) in a suspension culture generated according to the method of some embodiments of the invention.

According to one embodiment, there is provided a cell culture comprising MSCs and the culture medium of some embodiments of the invention.

According to one embodiment, there is provided a cell culture comprising MSCs and a defined culture medium comprising a basal medium and an effective amount of a knockout serum replacement (KoSR) and ITS (Insulin, Transferrin and Selenium), wherein the culture medium is serum-free.

According to one embodiment, there is provided a cell culture comprising MSCs and a defined culture medium comprising a basal medium comprising DMEM/F12, knockout serum replacement (KoSR) at a concentration of at least about 5%, and ITS (Insulin, Transferrin and Selenium) at a concentration of at least about 0.5%, wherein the culture medium is serum-free. According to a specific embodiment, this culture medium further comprises bFGF (e.g. FGF2) at a concentration of at least about 1 ng/ml.

According to one embodiment, there is provided a cell culture comprising MSCs and a culture medium comprising a basal medium comprising DMEM/F12 and MEM alpha at a volume ratio ranging between 0.4 to 2.3 DMEM/F12 to MEM alpha.

According to one embodiment, there is provided a cell culture comprising MSCs and a culture medium which comprises a basal medium comprising DMEM/F12 and MEM alpha at a volume ratio ranging between 0.6 to 1.5 DMEM/F12 to MEM alpha, a knockout serum replacement (KoSR) at a concentration of at least about 3%, ITS (Insulin, Transferrin and Selenium) at a concentration of at least about 0.1%, and serum at a concentration of at least about 5%.

According to one embodiment, there is provided a cell culture comprising PSCs and the culture medium of some embodiments of the invention.

According to one embodiment, there is provided a cell culture comprising PSCs and a culture medium comprising a basal medium comprising MEM alpha, serum and ITS (Insulin, Transferrin and Selenium).

According to one embodiment, there is provided a cell culture comprising PSCs and a culture medium comprising a basal medium comprising MEM alpha, serum at a concentration of at least 5%, and ITS (Insulin, Transferrin and Selenium) at a concentration of at least 1%.

According to one embodiment, there is provided a cell culture comprising PSCs and a defined culture medium comprising a basal medium and an effective amount of a knockout serum replacement (KoSR) and ITS (Insulin, Transferrin and Selenium), wherein the culture medium is serum-free.

According to one embodiment, there is provided a cell culture comprising PSCs and a defined culture medium comprising a basal medium comprising DMEM/F12, knockout serum replacement (KoSR) at a concentration of at least about 5%, and ITS (Insulin, Transferrin and Selenium) at a concentration of at least about 0.5%, wherein the culture medium is serum-free. According to a specific embodiment, this culture medium further comprises bFGF (e.g. FGF2) at a concentration of at least about 1 ng/ml.

According to one embodiment, there is provided a cell culture comprising PSCs and a culture medium comprising a basal medium comprising DMEM/F12 and MEM alpha at a volume ratio ranging between 0.4 to 2.3 DMEM/F12 to MEM alpha.

According to one embodiment, there is provided a cell culture comprising PSCs and a culture medium which comprises a basal medium comprising DMEM/F12 and MEM alpha at a volume ratio ranging between 0.6 to 1.5 DMEM/F12 to MEM alpha, a knockout serum replacement (KoSR) at a concentration of at least about 3%, ITS (Insulin, Transferrin and Selenium) at a concentration of at least about 0.1%, and serum at a concentration of at least about 5%.

The mesenchymal stem cells of some embodiments of the invention or the cell cultures of some embodiments of the invention can be utilized to produce high amounts (massive production) of proteins such as hormones, cytokines, growth factors and drugs. For example, to produce the proteins the cells should be induced to over-express the protein by transfection for example, and after expansion the protein could be isolated from the culture medium.

The mesenchymal stem cells of some embodiments of the invention or the cell cultures of some embodiments of the invention can be used to screen for factors (such as small molecule drugs, peptides, polynucleotides, and the like) or conditions (such as culture conditions or manipulation) that affect the differentiation of lineage precursor to terminally differentiated cells (e.g., for drug screening). For example, growth affecting substances, toxins or potential differentiation factors can be tested by their addition to the culture medium.

The mesenchymal stem cells of some embodiments of the invention or the cell cultures of some embodiments of the invention can be used to prepare a vaccine. For example, the pluripotent stem cells, mesenchymal stem cells or cells differentiated therefrom, can be inoculated with viral particles and further cultured in a suitable medium until cell lysis occurs and newly produced viral particles are released in the medium. The cells can be used for production of attenuated virus belonging to the family of poxvirus, in particular canarypoxvirus, fowlpoxvirus and vaccinia virus such as native or recombinant vaccinia virus [for example, Modified Vaccinia virus Ankara such as MVA available under ATCC Number VR-1508) or other orthopoxviruses]. For additional description see U.S. Patent Application No. 20040058441 which is fully incorporated herein by reference.

The pluripotent stem cells of some embodiments of the invention or the mesenchymal stem cells can be subject to genetic manipulation by using either infection or transfection of a polynucleotide of interest. The polynucleotide may be included in a nucleic acid construct under the regulation of a promoter.

Methods of introducing the polynucleotide into cells are described in Sambrook et al., [Molecular Cloning: A Laboratory Manual, Cold Springs Harbor Laboratory, New York (1989, 1992)]; Ausubel et al., [Current Protocols in Molecular Biology, John Wiley and Sons, Baltimore, Md. (1989)]; Chang et al., [Somatic Gene Therapy, CRC Press, Ann Arbor, Mich. (1995)]; Vega et al., [Gene Targeting, CRC Press, Ann Arbor Mich. (1995)]; Vectors [A Survey of Molecular Cloning Vectors and Their Uses, Butterworths, Boston Mass. (1988)] and Gilboa et al. [Biotechniques 4 (6): 504-512 (1986)] and include, for example, stable or transient transfection, lipofection, electroporation and infection with recombinant viral vectors [e.g., using retrovirus, adenovirus (e.g., adenovirus-derived vector Ad-TK, Sandmair et al., 2000. Hum Gene Ther. 11:2197-2205), a chimeric adenovirus/retrovirus vector which combines retroviral and adenoviral components (Pan et al., Cancer Letters 184: 179-188, 2002). See also U.S. Pat. No. 4,866,042 for vectors involving the central nervous system and also U.S. Pat. Nos. 5,464,764 and 5,487,992 for positive-negative selection methods for inducing homologous recombination.

The invention envisages the use of the expanded MSCs and/or differentiated MSCs of some embodiments of the invention for treating a disorder requiring cell replacement therapy (cell based therapy).

For example, mesenchymal cells can be used in treatment of bone and cartilage defects, for cardiovascular diseases, neurological diseases, and immunological diseases (as discussed in Parekkadan and Milwid, Mesenchymal Stem Cells as Therapeutics, Annu Rev Biomed Eng. (2010) 12: 87-117).

The mesenchymal stem cells of some embodiments of the invention can be administered to an organism per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the mesenchymal stem cells accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For example, for injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

Pharmaceutical compositions suitable for use in context of some embodiments of the invention include compositions wherein the active ingredients are contained in an amount effective to achieve the intended purpose. More specifically, a therapeutically effective amount means an amount of active ingredients (mesenchymal stem cells) effective to prevent, alleviate or ameliorate symptoms of a disorder (e.g., bone and cartilage defects) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide the active ingredient at a sufficient amount to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

The term “ng” refers to nanogram. The term “pg” refers to picogram. The term “ml” refers to milliliter. The term “mM” refers to millimolar. The term “μM” refers to micromolar.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions, illustrate the invention in a non-limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### General Materials and Experimental Procedures

Induced Pluripotent Stem (iPS) Cell Lines

KYOU-DXR0109B Human Induced Pluripotent Stem (IPS) Cells [201B7], ATCC Cat No. ACS-1023, were used.

DYR0100, Human Induced pluripotent stem (iPS) cells, ATCC, Cat #ACS-1011

Human Embryonic Stem Cell (hESC) Lines

I3 (TE03), ESC, Female cells were used

Mesenchymal Stem Cell (MSC) Lines

The following cells were used:

hMSC-BM-c PromoCell, Cat. No. C-12974

hMSC-UC-c PromoCell, Cat. No. C-12971.

hMSC-Adipo-c PromoCell, Cat. No. C-12977

Cell Culture/Mesenchymal Differentiation in Three-Dimensional Culture

hPSCs were cultured as cell aggregates or as single cells in suspension culture (three-dimensional, 3D) with serum-free media. Cell aggregates were passaged every 3-7 days mechanically by gentle pipetting or enzymatically using Trypsin EDTA (Sigma) or any equivalent enzyme.

Mesenchymal differentiation was induced by changing the media composition from hPSCs growth media to MSC differentiating media (see the list of differentiation media, Tables 6-9, below). The cells were passaged every 3-7 days mechanically by gentle pipetting or enzymatically using Trypsin EDTA (Sigma) or any equivalent enzyme. Following 2-21 days of differentiation, the surface expression of CD105, CD146, CD73, CD90, CD44, CD31 and/or CD45 was examined. MSC differentiation was typically determined by expression/high expression of CD105, CD146, CD73, CD90 and/or CD44 and low/no expression of CD31, CD34 and/or CD45. Additionally, expression of key pluripotency markers (e.g. Oct4 and Nanog) was examined indicating the loss of pluripotency through mesenchymal differentiation.

The resultant MSC cells, which were differentiated from hESCs or iPSCs, termed “hESC-MSCs” or “iPSC-MSCs”, respectively, hereinafter, were plastic adherent when re-plated in 2D adherent culture. The hESC-MSCs further differentiated into osteocytes, adipocytes in 2D culture and chondrocytes in 3D pellet culture.

Adipogenic Differentiation

hESC-MSCs from 3D culture were seeded in 2D culture and grown in a DMEM F-12 media containing 10% FBS and 2 mM 1-glutamine. The media was supplemented with 0.5 mM 3-isobutyl-1-methylxanthine, 10 mg/mL insulin, 10⁻⁶ M dexamethasone, and 0.1 mM indomethacin (all from Sigma-Aldrich). The media was replaced every 3-4 days for up to 4 weeks. Adipogenic differentiation was confirmed by Oil Red O staining (Sigma Aldrich), discussed below.

Osteogenic Differentiation

hESC-MSCs from 3D culture were seeded in 2D culture and grown in an osteogenic media comprised of GMEM BHK-21 (Gibco BRL), with 10% FBS, 1 mM sodium pyruvate (Gibco BRL), 1% nonessential amino acids, 50 mg/mL L-ascorbic acid (Sigma), 0.075 mM b-mercaptoethanol, 10 mM b-glycerol-phosphate and 0.1 mM dexamethasone (all from Sigma). The media was replaced every 3-4 days for 3-4 weeks. Osteogenic differentiation was confirmed by Alizarin red staining, discussed below.

Chondrogenic Differentiation

hESC-MSCs from 3D culture were (2×10⁵ cells) were centrifuged at 300 g for 5 min in 15-mL polypropylene falcon tubes to form a cell pellet. The cells were grown for 4-9 weeks in the DMEM (Gibco BRL), supplemented with 10⁻⁷ M dexamethasone, 1% ITS, 50 mg/mL L-ascorbic acid, 1 mM sodium pyruvate, 4 mM 1-proline (Biological Industries, also referred to as BI), and 10 ng/mL TGFβ3 (R&D systems). The media was replaced twice each week without disturbing the cell mass. Chondrogenic differentiation was confirmed by Hematoxylin and eosin (H&E) and Alcian blue staining, discussed below.

Cell Culture—Adult and Fetal MSCs

Adult and fetal MSCs were cultured at least for 3 passages in growth media (see the list of growth media, Tables 1-4 and 5A-C, below) in 2D culture (with or w/o plate coating). The cells were enzymatically passaged every 3-7 days using Trypsin EDTA (Sigma) or any equivalent enzyme.

Alternatively, hESC-MSCs or iPSC-MSCs were cultured in suspension cultures (3D) in growth media (see the list of growth media, Tables 1-4 and 5A-C, below) for 7-30 days. Cells were passaged mechanically by gentle pipetting or enzymatically using Trypsin EDTA (Sigma) or any equivalent enzyme.

The cells were characterized for marker expression (expression of CD105, CD146, CD73, CD90 and/or CD44 and low/no expression of CD31, CD34 and/or CD45) and multilineage differentiation potential towards adipocytes, osteocytes and chondrocytes.

In all experiments growth factors (GFs) were added freshly in each medium change.

List of Growth Media for Adult and Fetal MSCs

The following culture medium combinations were tested for their ability to support the growth (cell proliferation/expansion) of MSCs in 2D cultures or in suspension cultures (3D):

TABLE 1A
MSC basal medium comprising DMEM/FT2
(w/o bFGF or other growth factors)
MSC basal medium (MSC-Diff 0-F12)

| Reagent name, Company, | |
|---|---|
| Catalog No. | Final Concentration |
| DMEM/F12 | 90% |
| BI, Cat: 01-170-1A | |
| KoSR | 7.5% |
| Gibco, Cat: 10828-028 | |
| ITS | 0.5% |
| Gibco, Cat: 41400-045 | |
| NEAA | 0.5% |
| Gibco, Cat: 11140-035 | |
| β-mercaptoethanol | 0.05 mM |
| Gibco, Cat: 31350-010 | |
| L-glutamine | 1 mM |
| BI, Cat: 03-020-1B | |
| Penicillin-Streptomycin Solution | Penicillin: 50 U/ml |
| BI, Cat: 03-031-1B | Streptomycin: 0.05 mg/ml |

TABLE 1B
MSC basal medium comprising
DMEM high glucose (DMEM HG)
(w/o bFGF orother growth factors)
MSC basal medium (MSC-Diff 0-HG)

| Reagent name, Company, | |
|---|---|
| Catalog No. | Final Concentration |
| DMEM HG | 90% |
| BI, Cat: 01-052-1A | |
| KoSR | 7.5% |
| Gibco, Cat: 10828-028 | |
| ITS | 0.5% |
| Gibco, Cat: 41400-045 | |
| NEAA | 0.5% |
| Gibco, Cat: 11140-035 | |
| β-mercaptoethanol | 0.05 mM |
| Gibco, Cat: 31350-010 | |
| L-glutamine | 1 mM |
| BI, Cat: 03-020-1B | |
| Penicillin-Streptomycin Solution | Penicillin: 50 U/ml |
| BI, Cat: 03-031-1B | Streptomycin: 0.05 mg/ml |

TABLE 2
MSC growth media (based on MSC-Diff 0-FT2
or MSC-Diff 0-HG + growth factors)-Part I

| | Concentration of GFs in | | |
|---|---|---|---|
| | MSC growth medium | | |
| | bFGF | EGF | PDGF-BB |
| Medium Name | (ng/ml) | (ng/ml) | (ng/ml) |
| MSC-Diff 0-F12 | — | — | — |
| or | | | |
| MSC-Diff 0-HG | | | |
| MSC-Diff 1 | 1 | — | — |
| MSC-Diff 2 | 5 | — | — |
| MSC-Diff 3 | 10 | — | — |
| MSC-Diff 4 | 20 | — | — |
| MSC-Diff 5 | 50 | — | — |
| MSC-Diff 6 | — | — | 10 |
| MSC-Diff 7 | 1 | — | 10 |
| MSC-Diff 8 | 20 | — | 10 |
| MSC-Diff 9 | — | — | 25 |
| MSC-Diff 10 | 1 | — | 25 |
| MSC-Diff 11 | 20 | — | 25 |
| MSC-Diff 12 | 1 | 10 | — |
| MSC-Diff 13 | 20 | 10 | — |
| MSC-Diff 14 | 1 | 20 | — |
| MSC-Diff 15 | 20 | 20 | — |

TABLE 3
MSC growth media (based on MSC-Diff 0-FT2
or MSC-Diff 0-HG + growth factors)-Part II

| | Concentration of GFs in MSC growth medium | | | | |
|---|---|---|---|---|---|
| | bFGF | FGF4 | TGFβ1 | TGFβ3 | WNT-3a |
| Medium Name | (ng/ml) | (ng/ml) | (ng/ml) | (ng/ml) | (ng/ml) |
| MSC-Diff 0-F12 | — | — | — | — | — |
| or | | | | | |
| MSC-Diff 0-HG | | | | | |
| MSC-Diff 16 | 1 | 1 | — | — | — |
| MSC-Diff 17 | 1 | 5 | — | — | — |
| MSC-Diff 18 | 1 | 10 | — | — | — |
| MSC-Diff 19 | 5 | 1 | — | — | — |
| MSC-Diff 20 | 5 | 5 | — | — | — |
| MSC-Diff 21 | 5 | 10 | — | — | — |
| MSC-Diff 22 | 10 | 1 | — | — | — |
| MSC-Diff 23 | 10 | 5 | — | — | — |
| MSC-Diff 24 | 10 | 10 | — | — | — |
| MSC-Diff 25 | 20 | 1 | — | — | — |
| MSC-Diff 26 | 20 | 5 | — | — | — |
| MSC-Diff 27 | 20 | 10 | — | — | — |
| MSC-Diff 28 | 50 | 1 | — | — | — |
| MSC-Diff 29 | 50 | 5 | — | — | — |
| MSC-Diff 30 | 50 | 10 | — | — | — |
| MSC-Diff 31 | 50 | — | 5 | — | — |
| MSC-Diff 32 | 50 | — | 10 | — | — |
| MSC-Diff 33 | 50 | — | — | 5 | — |
| MSC-Diff 34 | 50 | — | — | 10 | — |
| MSC-Diff 40 | 1 | — | — | — | 10 |
| MSC-Diff 41 | 1 | — | — | — | 100 |
| MSC-Diff 42 | 5 | — | — | — | 10 |
| MSC-Diff 43 | 5 | — | — | — | 100 |
| MSC-Diff 44 | 10 | — | — | — | 10 |
| MSC-Diff 45 | 10 | — | — | — | 100 |
| MSC-Diff 46 | 20 | — | 5 | — | — |
| MSC-Diff 47 | 20 | — | 10 | — | — |
| MSC-Diff 48 | 20 | — | — | 5 | — |
| MSC-Diff 49 | 20 | — | — | 10 | — |
| MSC-Diff 50 | — | — | — | — | 10 |

TABLE 4
Growth factors used in MSC growth media
(per tables 2 and 3 above)

| Reagent name, Company, | | |
|---|---|---|
| Catalog No. | Final Concentration | |
| bFGF stock solution | 1, 5, 10, 20 and 50 | ng/ml |
| R&D, Cat: 233-FB | | |
| Recombinant Human EGF | 10 and 20 | ng/ml |
| Peprotech, Cat: AF-100-15 | | |
| Recombinant Human PDGF-BB | 10 and 25 | ng/ml |
| Peprotech, Cat: 100-14B | | |
| FGF4 | 1-10 | ng/ml |
| Peprotech, Cat: 100-31 or | | |
| R&D systems, Cat: 7460-F4-025/CF | | |
| TGFβ1 | 5-10 | ng/ml |
| Peprotech, Cat: 100-21 or | | |
| R&D systems, Cat: 240-B/CF | | |
| TGFβ3 | 5-10 | ng/ml |
| Peprotech, Cat: 100-36E or | | |
| R&D systems, Cat: 243-B3 | | |
| WNT-3a | 10-200 | ng/ml |

R&D systems, Cat: 5036-WN/CF

TABLE 5A
An additional MSC growth medium
MSC growth medium (MSC-Diff 35)

| Reagent name, Company, | |
|---|---|
| Catalog No. | Final Concentration |
| DMEM/F12 | 42% |
| BI, Cat: 01-170-1A | |
| MEM Alpha medium | 42% |
| BI, Cat: 01-043-1A | |
| FBS | 10% |
| BI, Cat: 04-007-1A | |
| KoSR | 3.75% |
| Gibco, Cat: 10828-028 | |
| ITS | 0.25% |
| Gibco, Cat: 41400-045 | |
| bFGF stock solution | 25 ng/ml |
| R&D, Cat: 233-FB | |
| NEAA | 1% |
| Gibco, Cat: 11140-035 | |
| β-mercaptoethanol | 0.05 mM |
| Gibco, Cat: 31350-010 | |
| L-glutamine | 1 mM |
| BI, Cat: 03-020-1B | |
| Penicillin-Streptomycin Solution | Penicillin: 50 U/ml |
| BI, Cat: 03-031-1B | Streptomycin: 0.05 mg/ml |

TABLE 5B
An additional MSC growth medium
MSC growth medium (MSC-Diff 51)

| Reagent name | Final Concentration |
|---|---|
| DMEM/F12 | 90% |
| BI, Cat: 01-170-1A | |
| KoSR | 7.5% |
| Gibco, Cat: 10828-028 | |
| ITS | 0.5% |
| Gibco, Cat: 41400-045 | |
| NEAA | 0.5% |
| Gibco, Cat: 11140-035 | |
| β-mercaptoethanol | 0.05 mM |
| Gibco, Cat: 31350-010 | |
| bFGF (stock solution) | 50 ng/mL |
| R&D, Cat: 233-FB | |
| L-ascorbic acid | 50 μg/mL |
| Sigma Aldrich Cat: A4544 | |
| L-glutamine | 1 mM |
| BI, Cat: 03-020-1B | |
| Penicillin-Streptomycin Solution | Penicillin: 50 U/ml |
| BI, Cat: 03-031-1B | Streptomycin: 0.05 mg/ml |

TABLE 5C
An additional MSC growth medium
MSC growth medium (MSC-Diff 52)

| Reagent name | Final Concentration |
|---|---|
| DMEM HG | 90% |
| BI, Cat: 01-052-1A | |
| KoSR | 7.5% |
| Gibco, Cat: 10828-028 | |
| ITS | 0.5% |
| Gibco, Cat: 41400-045 | |
| NEAA | 0.5% |
| Gibco, Cat: 11140-035 | |
| β-mercaptoethanol | 0.05 mM |
| Gibco, Cat: 31350-010 | |
| bFGF (stock solution) | 50 ng/mL |
| R&D, Cat: 233-FB | |
| L-ascorbic acid | 50 μg/mL |
| Sigma, Cat: A4544 | |
| Sodium pyruvate | 110 μg/mL |
| BI, 03-042-1B | |
| L-glutamine | 1 mM |
| BI, Cat: 03-020-1B | |
| Penicillin-Streptomycin Solution | Penicillin: 50 U/ml |
| BI, Cat: 03-031-1B | Streptomycin: 0.05 mg/ml |

List of MSC Differentiation Media

The following culture media were tested for their ability to support differentiation of human pluripotent stem cells (hPSCs) to MSCs in suspension cultures (3D)):

TABLE 6
Differentiation medium for differentiation of hPSCs towards MSCs
MSC-Diff 36 medium

| Reagent name, Company, | |
|---|---|
| Catalog No. | Final Concentration |
| DMEM/F12 | 90% |
| BI, Cat: 01-170-1A | |
| KoSR | 7.5% |
| Gibco, Cat: 10828-028 | |
| ITS | 0.5% |
| Gibco, Cat: 41400-045 | |
| bFGF stock solution | 50 ng/ml |
| R&D, Cat: 233-FB | |
| NEAA | 0.5% |
| Gibco, Cat: 11140-035 | |
| β-mercaptoethanol | 0.05 mM |
| Gibco, Cat: 31350-010 | |
| L-glutamine | 1 mM |
| BI, Cat: 03-020-1B | |
| Penicillin-Streptomycin Solution | Penicillin: 50 U/ml |
| BI, Cat: 03-031-1B | Streptomycin: 0.05 mg/ml |

* Of note, L-glu and Pen-Strep together are 1% of the final medium

TABLE 7
Differentiation medium for differentiation of hPSCs towards MSCs
MSC-Diff 37 medium

| Reagent name, Company, | |
|---|---|
| Catalog No. | Final Concentration |
| DMEM/F12 | 42% |
| BI, Cat: 01-170-1A | |
| MEM Alpha medium | 42% |
| BI, Cat: 01-043-1A | |
| FBS | 10% |
| BI, Cat: 04-007-1A | |
| KoSR | 3.75% |
| Gibco, Cat: 10828-028 | |
| ITS | 0.25% |
| Gibco, Cat: 41400-045 | |
| bFGF stock solution | 25 ng/ml |
| R&D, Cat: 233-FB | |
| NEAA | 1% |
| Gibco, Cat: 11140-035 | |
| β-mercaptoethanol | 0.05 mM |
| Gibco, Cat: 31350-010 | |
| L-glutamine | 1 mM |
| BI, Cat: 03-020-1B | |
| Penicillin-Streptomycin Solution | Penicillin: 50 U/ml |
| BI, Cat: 03-031-1B | Streptomycin: 0.05 mg/ml |

* Of note, L-glu and Pen-Strep together are 1% of the final medium

TABLE 8
Differentiation medium for differentiation of hPSCs towards MSCs
MSC-Diff 38 medium

| Reagent name, Company, | |
|---|---|
| Catalog No. | Final Concentration |
| MEM Alpha medium | 92% |
| BI, Cat: 01-043-1A | |
| FBS | 5% |
| BI, Cat: 04-007-1A | |
| ITS | 1% |
| Gibco, Cat: 41400-045 | |
| NEAA | 1% |
| Gibco, Cat: 11140-035 | |
| L-glutamine | 1 mM |
| BI, Cat: 03-020-1B | |
| Penicillin-Streptomycin Solution | Penicillin: 50 U/ml |
| BI, Cat: 03-031-1B | Streptomycin: 0.05 mg/ml |

* Of note, L-glu and Pen-Strep together are 1% of the final medium

TABLE 9
Differentiation medium for differentiation of hPSCs towards MSCs
MSC-Diff 39 medium

| Reagent name, Company, | |
|---|---|
| Catalog No. | Final Concentration |
| MEM Alpha medium | 77% |
| BI, Cat: 01-043-1A | |
| FBS | 20% |
| BI, Cat: 04-007-1A | |
| ITS | 1% |
| Gibco, Cat: 41400-045 | |
| NEAA | 1% |
| Gibco, Cat: 11140-035 | |
| L-glutamine | 1 mM |
| BI, Cat: 03-020-1B | |
| Penicillin-Streptomycin Solution | Penicillin: 50 U/ml |
| BI, Cat: 03-031-1B | Streptomycin: 0.05 mg/ml |

* Of note, L-glu and Pen-Strep together are 1% of the final medium

Proliferation Assay

Once the cells in each experiment reached approximately 70-90% confluence, the cells were counted, and fold increase, population doubling time and cumulative cell counts were determined by population doubling time (PDT) or by colorimetric assay (PrestoBlue).

Flow Cytometry Analysis (FACS)

Cells were harvested using enzymatic solutions (for 2D cultures) or by centrifugation (for 3D cultures). Cells were resuspended in a staining buffer and incubated with each of the indicated antibodies: CD45, CD105, CD44, C146, CD31, CD34 (Miltenyi), CD73 (eBioscience), CD90 (BD Pharmingen), Oct3/4 and/or Nanog (Miltenyi) and acquired using BD™ LSR II flow cytometer.

Alizarin Red Staining

Cells were rinsed twice with PBS, fixed with 4% PFA for 20 minutes, rinsed again, and stained with 2% Alizarin red solution (pH 4.1-4.5; Sigma) for 15 minutes at room temperature and rinsed with water three times and left in water.

Oil Red Staining

Cells were rinsed three times with PBS, fixed with 4% paraformaldehyde (PFA; Electron Microscopy Sciences) for 20 minutes, rinsed again with PBS, and stained for 10 minutes at room temperature with an Oil Red O solution (Sigma Aldrich), then rinsed with water three times and left in water.

Alcian Blue Staining

Cell sections were prepared after fixing the cell pellets with 4% PFA and embedding in low melting point agarose (1.5%). Hematoxylin and eosin (H&E) and Alcian blue staining was conducted.

### Example 1

### Human Progenitor Stem Cells Differentiation Towards Mesenchymal Stem Cells in 3D Suspension Cultures

Human PSCs (lines: TE03, Kyou, 13) were cultured as aggregates (Maxells™) or as single cells (Singles™) in suspension culture system.

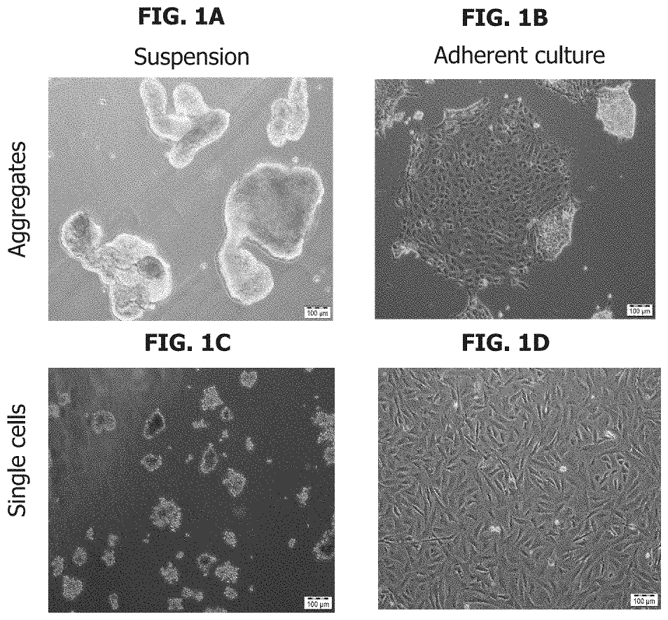

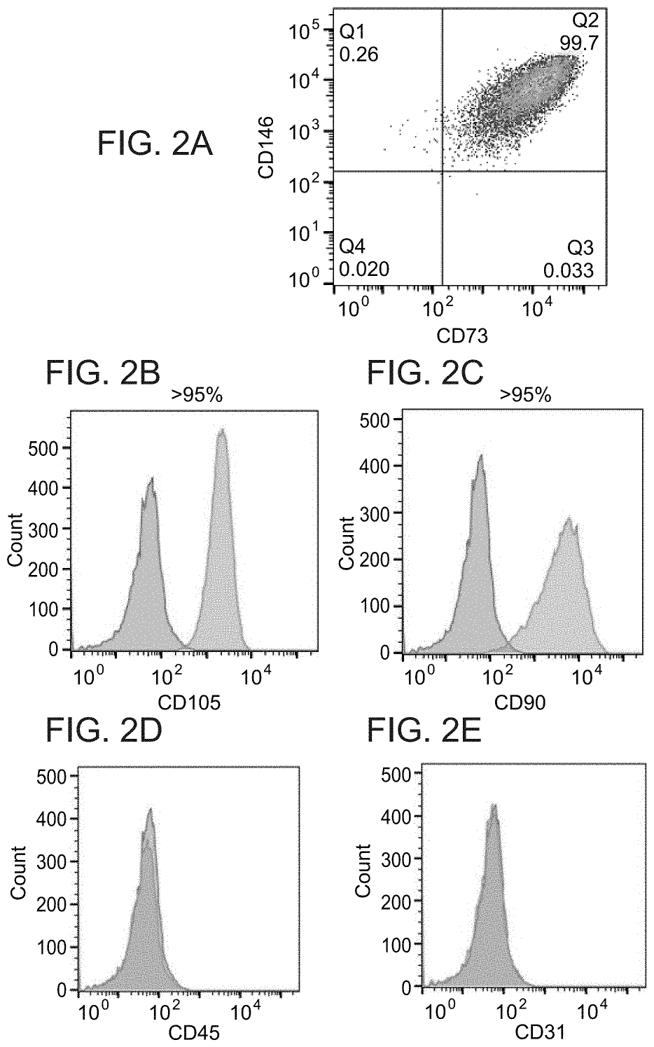

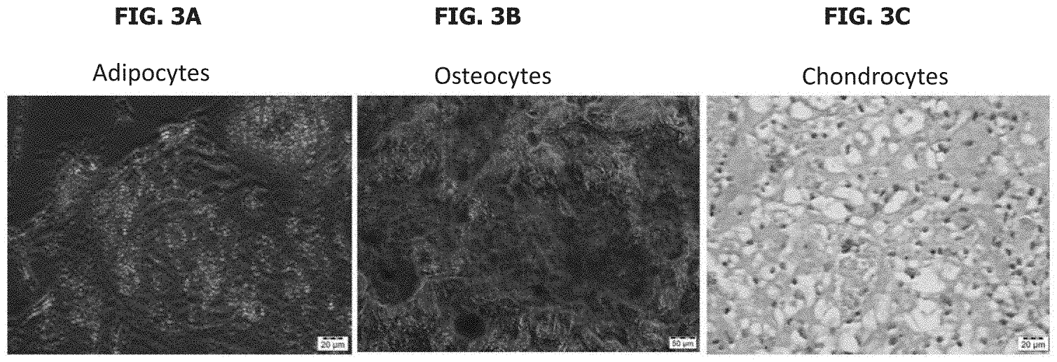

By changing the media from hPSCs basal growth media to MSC-Diff and high levels of 36 media, and following 2-21 days in suspension culture, the cells differentiated into MSCs as evident by morphology (FIGS. 1A and 1C) and by the co-expression of CD105, CD44, CD73, CD90 and high levels of CD146 (FIGS. 2A-C). Furthermore, the cells were devoid of the endothelial marker CD31 and the hematopoietic marker CD45 (FIGS. 2D-E). Furthermore, the cells were plastic-adherent (FIGS. 1B and 1D), and differentiated into mesodermal derivatives (osteoblasts, adipocytes and chondroblasts) in-vitro (FIGS. 3A-C).

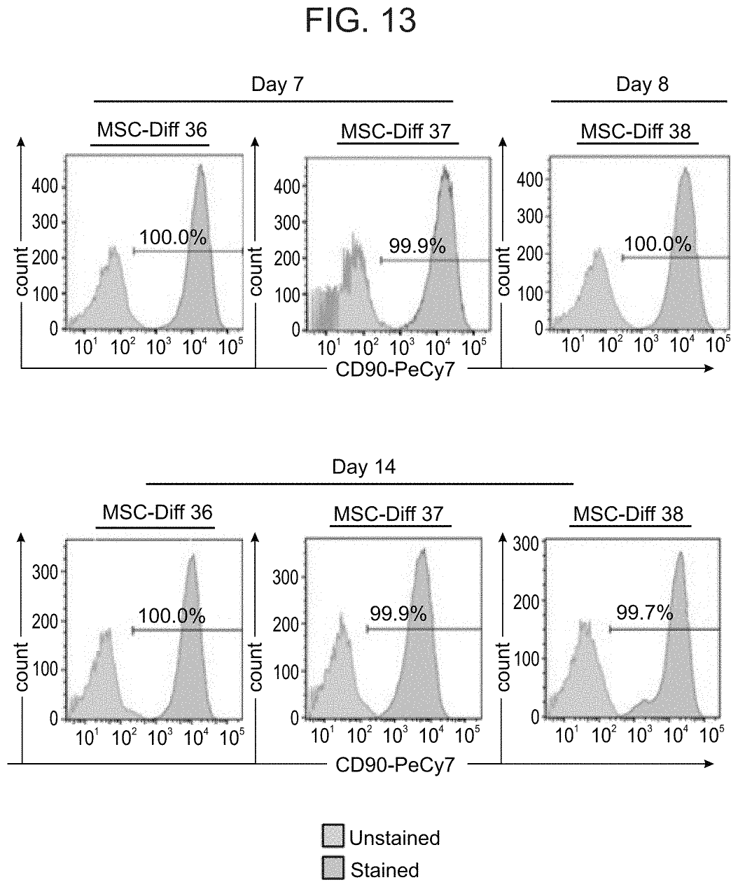

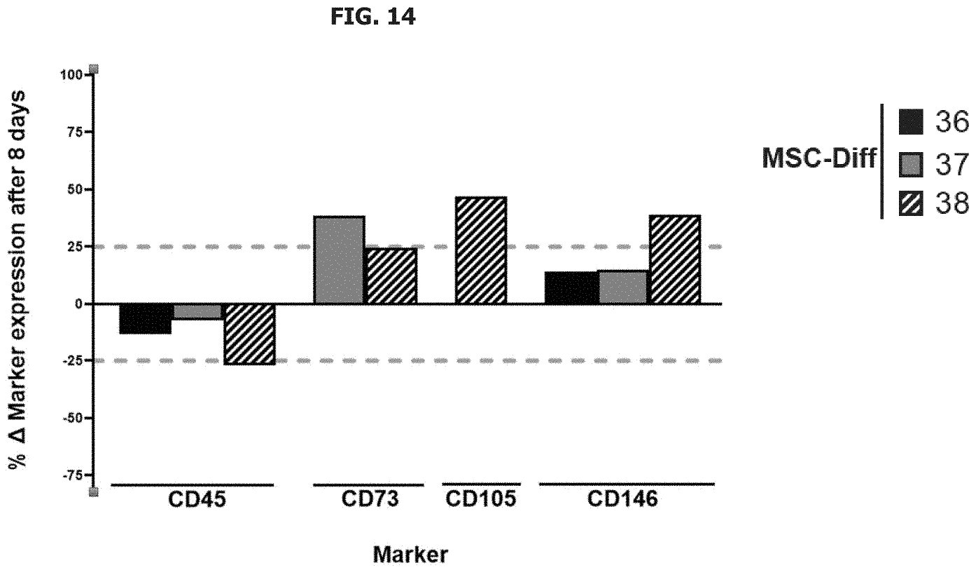

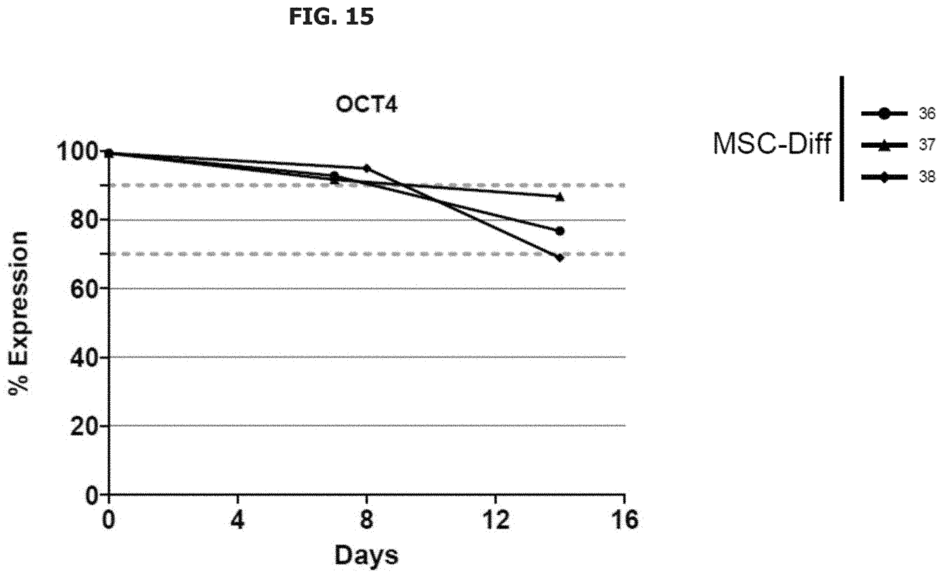

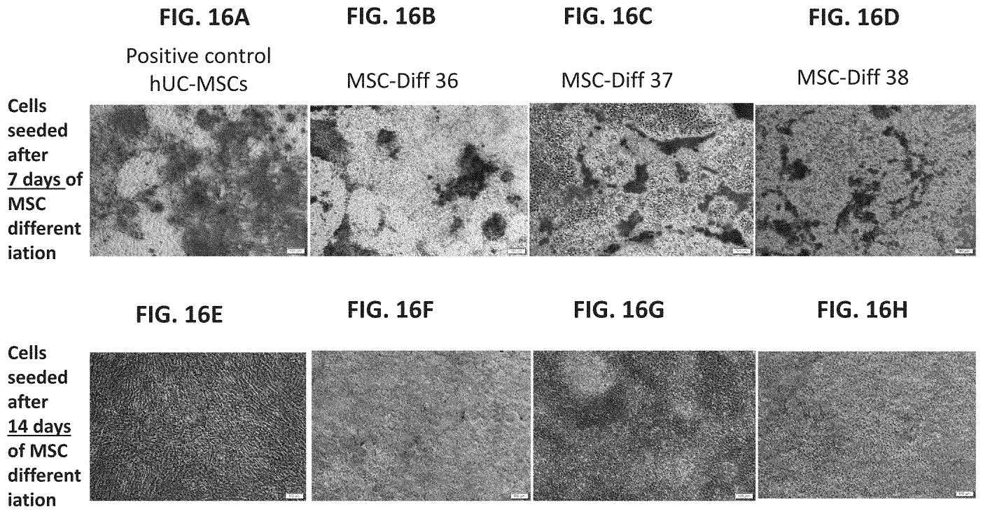

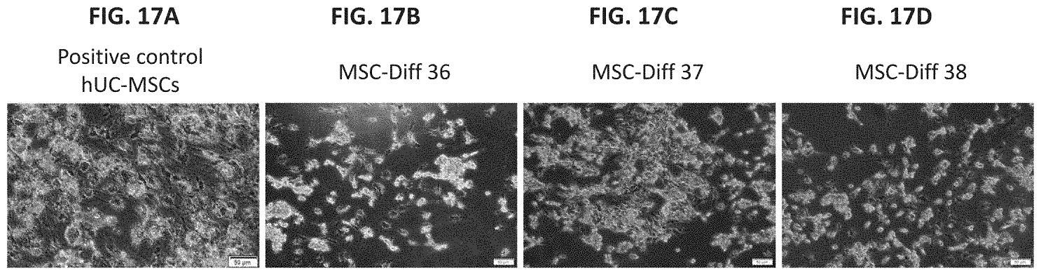

In a further experiment, human induced stem cells (iPS cell line Dyr0100) were grown in three different differentiation media, i.e. MSC-Diff 36, MSC-Diff 37 or MSC-Diff 38, and following 7-14 days in suspension culture, the cells were assessed for MSC cell signature. As evident from FIG. 13, the mesenchymal marker CD90 was evident 7-8 days following the beginning of culture with all three differentiation media. MSC differentiation was further evident in all three culture media by the increased expression of the mesenchymal markers: CD73, CD105 and CD146 and the decreased expression of the hematopoietic marker CD45 (FIG. 14), as well as by the significant decrease in the pluripotency marker OCT4 as mesenchymal differentiation progressed (FIG. 15). Furthermore, the iPSC-derived MSCs (following 7 or 14 days of mesenchymal differentiation in all three tested media) differentiated in-vitro into osteocytes (FIGS. 16A-H) and adipocytes (FIGS. 17A-D).

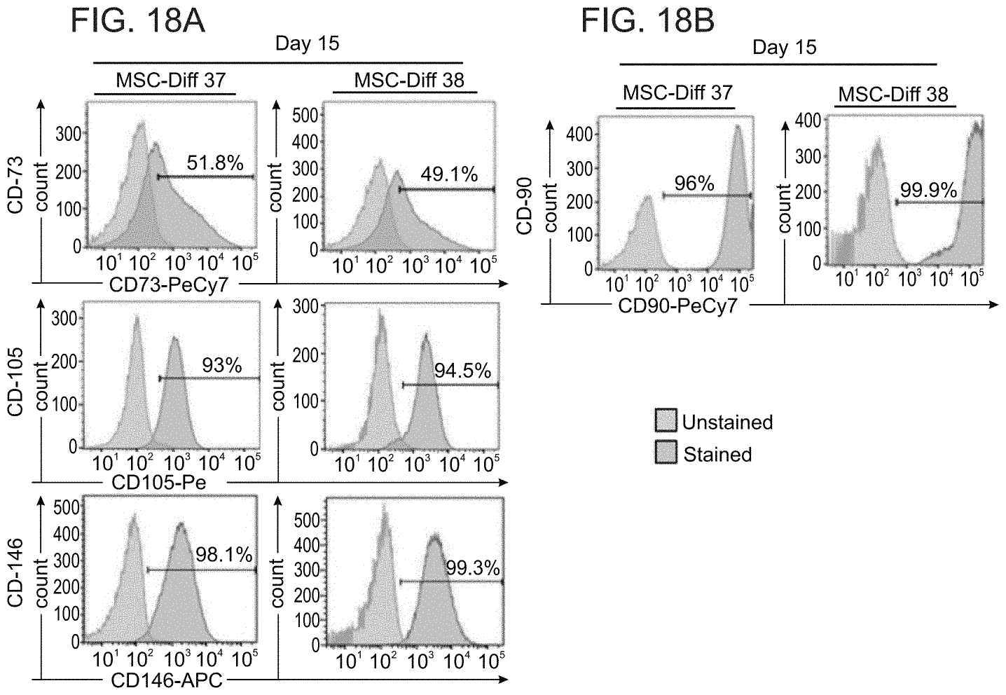

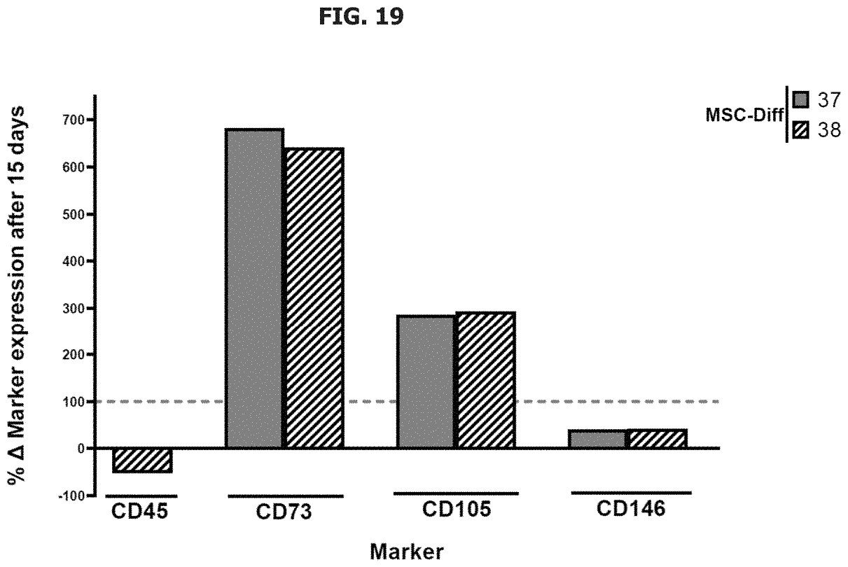

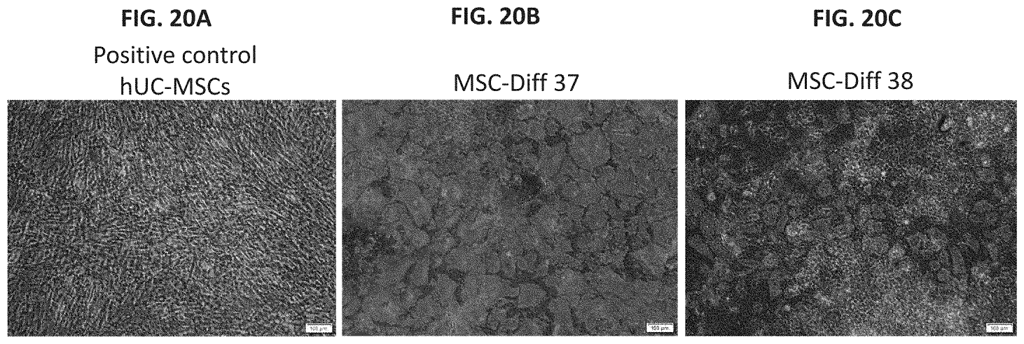

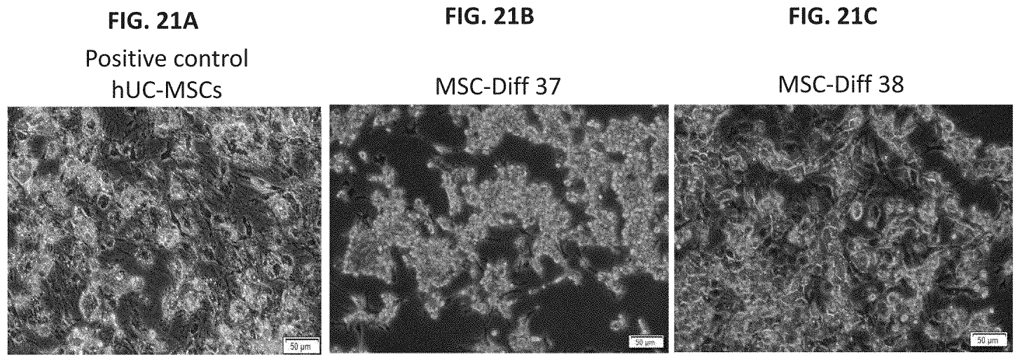

Similar results were evident for iPSC-derived MSCs following replating in 2D adherent cultures. Specifically, human iPS-derived MSCs differentiated in 3D suspension cultures with differentiation media MSC-Diff 37 and MSC-Diff 38 maintained high expression of the mesenchymal markers CD73, CD90, CD105 and CD146 after replating in 2D culture for 15 days (FIGS. 18A-B and 19) and presented low expression of the hematopoietic marker CD45 (FIG. 19). Furthermore, after replating in 2D cultures, these iPSC-derived MSCs differentiated in-vitro into osteocytes (FIGS. 20A-C) and adipocytes (FIGS. 21A-C).

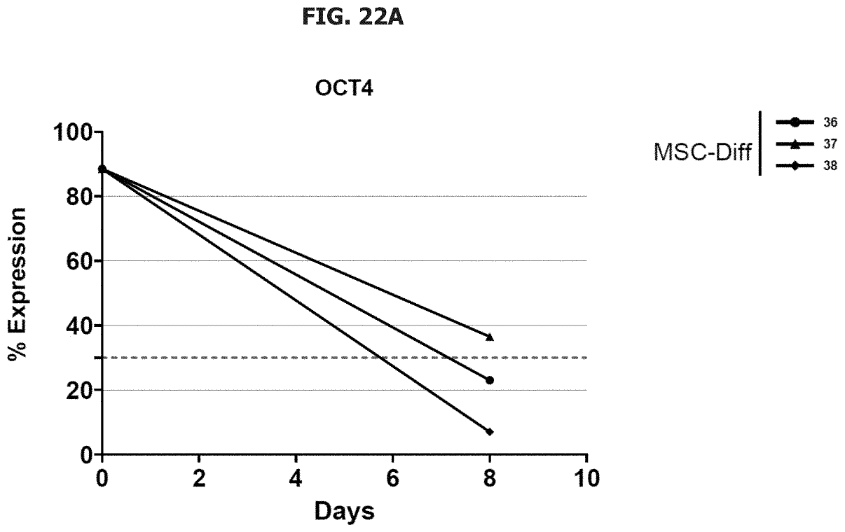

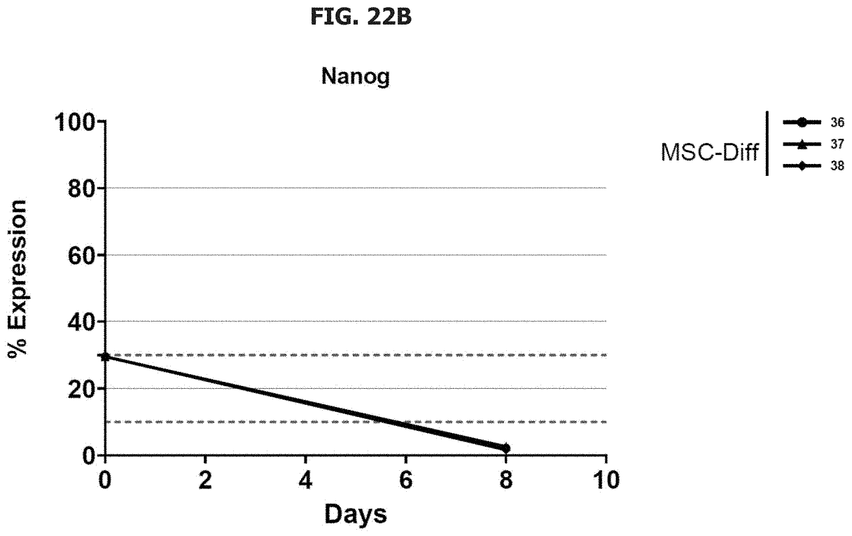

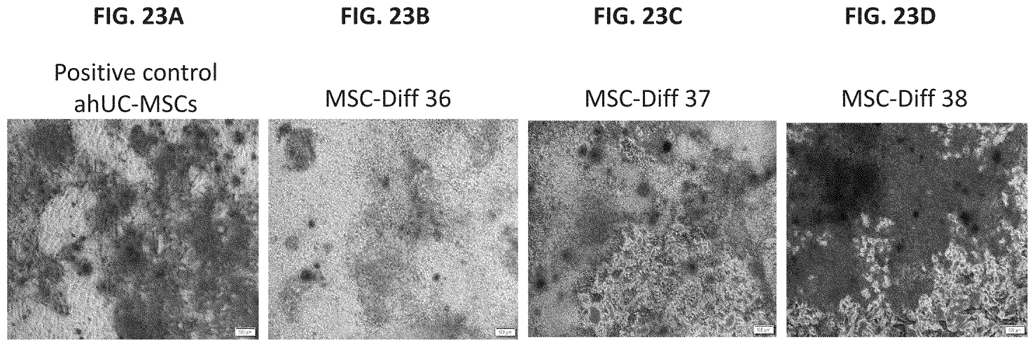

Similar results regarding 3D mesenchymal differentiation using MSC-Diff 36, MSC-Diff 37 and MSC-Diff 38 media were observed using ESCs. FIG. 22A and FIG. 22B demonstrate the decrease in the expression levels of Oct4 and Nanog in ESC-derived MSCs cells after 8 days of mesenchymal differentiation in 3D, respectively, while FIGS. 23A-D demonstrate positive Alizarin red staining indicating the osteogenic potential of ESC-derived MSCs.

### Example 2

### Expansion of Mesenchymal Stem Cells in Media Comprising Different Growth Factors

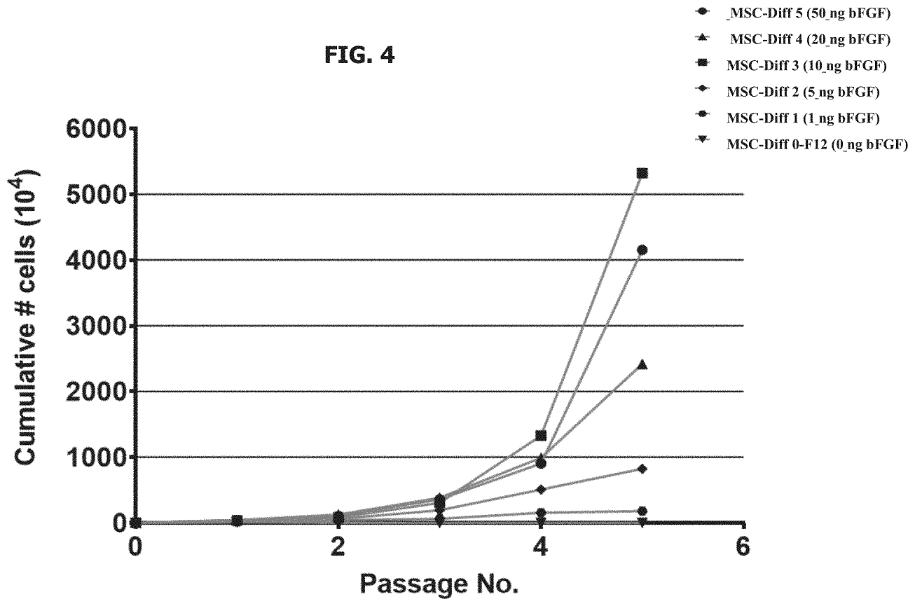

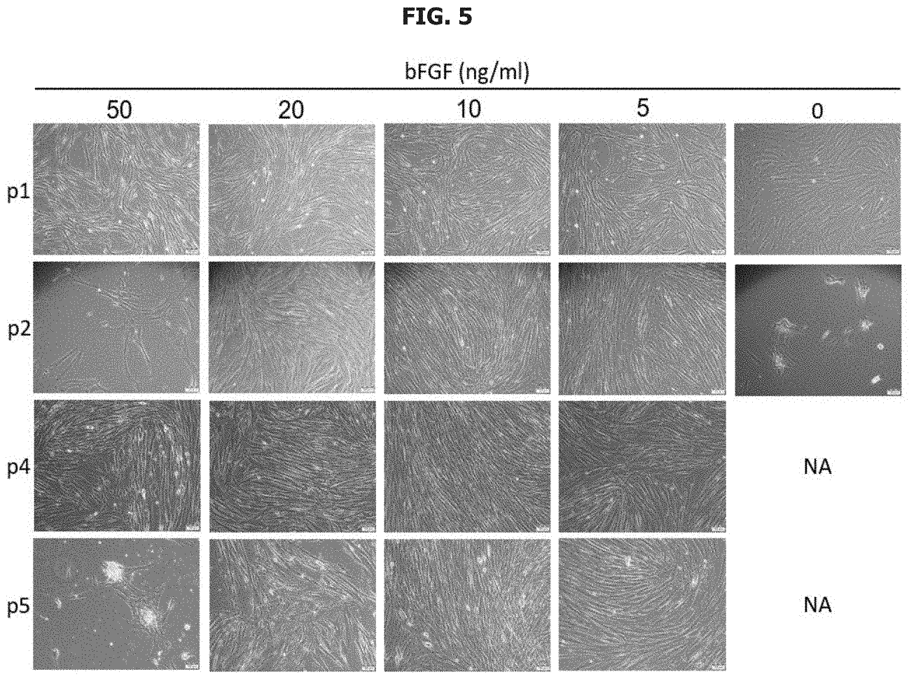

Adult human MSCs (obtained from bone marrow or umbilical cord) were cultured in MSC basal medium (MSC-diff 0-F12) comprising different concentrations of basic fibroblast growth factor (bFGF) (Table 10, below). As shown in FIGS. 4 and 5, bFGF is vital to support the growth and expansion of MSCs. Even when low concentrations of bFGF were used, e.g. 5 ng/ml, growth and expansion of MSCs was evident.

TABLE 10
Experimental design for expansion of MSCs in growth medium comprising different
concentrations of bFGF

| Experiment No. | 1 | 2 | 3 |
|---|---|---|---|
| Experiment was | duplicates | duplicates | triplicates |
| performed in | | | |
| No. of cells seeded/cm² | 5000-20,000 | 5000-20,000 | 5000-20,000 |
| Type of cells | Adult human | Adult human | Adult human |
| | MSC-BM | MSC-UC | MSC-UC |
| Passage at seeding at | 5 | 3 | 3 |
| start of experiment | | | |
| Number of cell passages | 2-3 | 5 | 5 |
| (=number of times cells | | | |
| were counted) | | | |
| bFGF concentration and | 1 ng (MSC-Diff 1) | 0 ng (MSC-Diff 0) | 0 ng (MSC-Diff 0) |
| medium name | 10 ng (MSC-Diff 3) | 1 ng (MSC-Diff 1) | 5 ng (MSC-Diff 2) |
| | 20 ng (MSC-Diff 4) | 10 ng (MSC-Diff 3) | 10 ng (MSC-Diff 3) |
| | 50 ng (MSC-Diff 5) | 20 ng (MSC-Diff 4) | 20 ng (MSC-Diff 4) |
| | | 50 ng (MSC-Diff 5) | 50 ng (MSC-Diff 5) |

Next, the growth and expansion of MSCs was examined in MSC basal medium (MSC-diff 0-F12) comprising different growth factors. Specifically, bFGF, epidermal growth factor (EGF) and/or platelet-derived growth factor-BB (PDGF-BB) were added at different concentrations and combinations to the MSC basal medium (MSC-Diff 0-F12), per Table 2 above, and their effect on cell proliferation, expansion and survival of adult hMSC-UC cells was examined. Thus, once the cells in each experiment reached approximately 70-90% confluence, the cells were counted, and fold increase, population doubling time and cumulative cell counts were determined.

In another set of experiments, bFGF, FGF4, transforming growth factor beta 1 or 3 (TGFβ 1 or TGFβ3, respectively), PDGF-BB, EGF and/or WNT-3a were added at different concentrations and combinations to the MSC basal medium (MSC-Diff 0-F12 or MSC-Diff 0-HG), per Tables 2 and 3, above, and their effect on cell proliferation, expansion and survival of adult hMSC-UC cells was examined. Thus, once the cells in each experiment reached approximately 70-90% confluence, the cell proliferation was estimated according to a colorimetric assay (PrestoBlue).

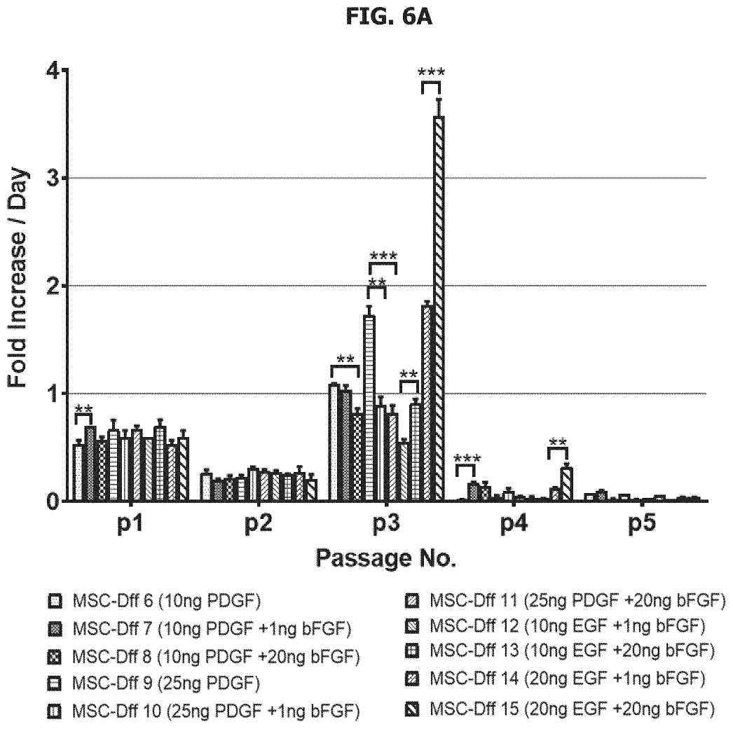

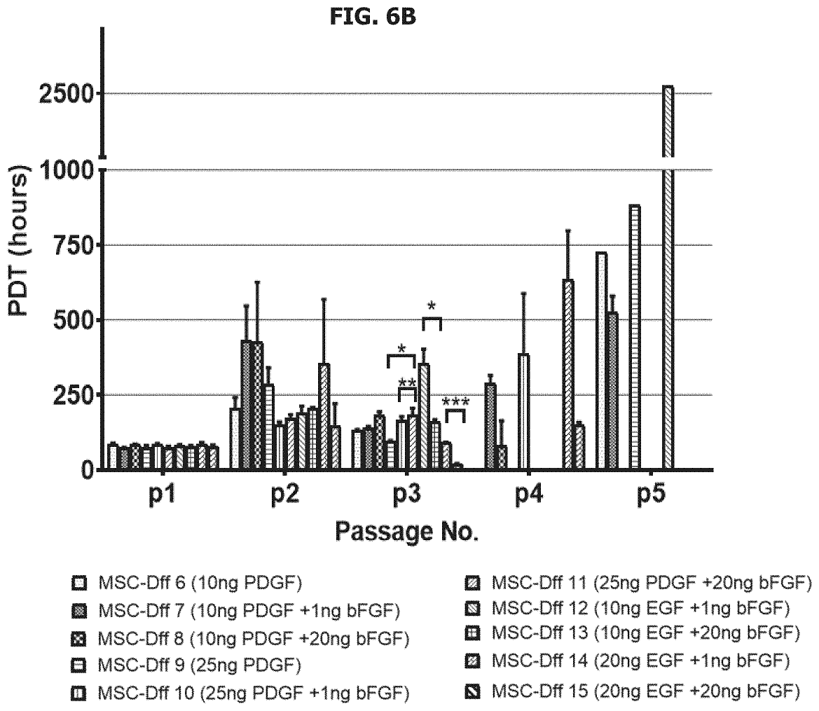

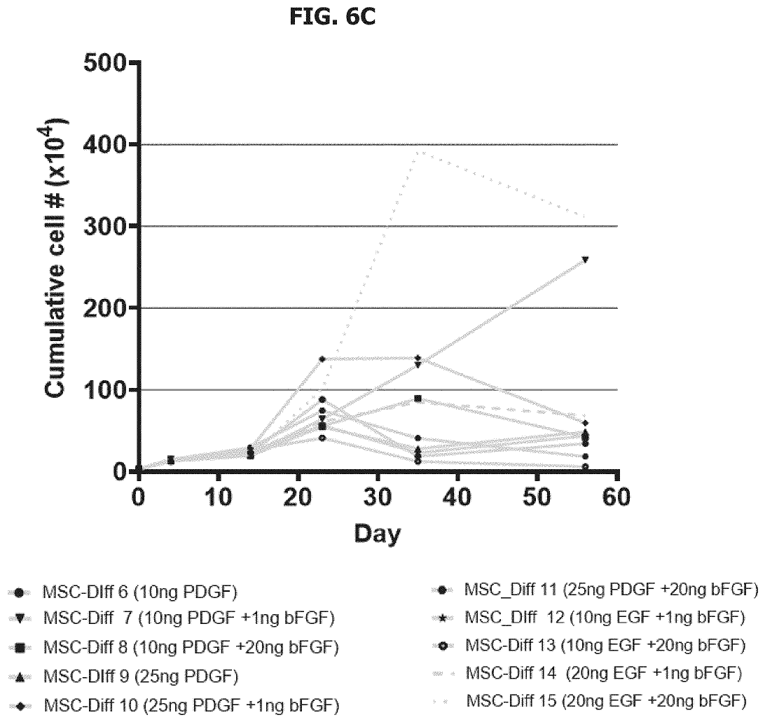

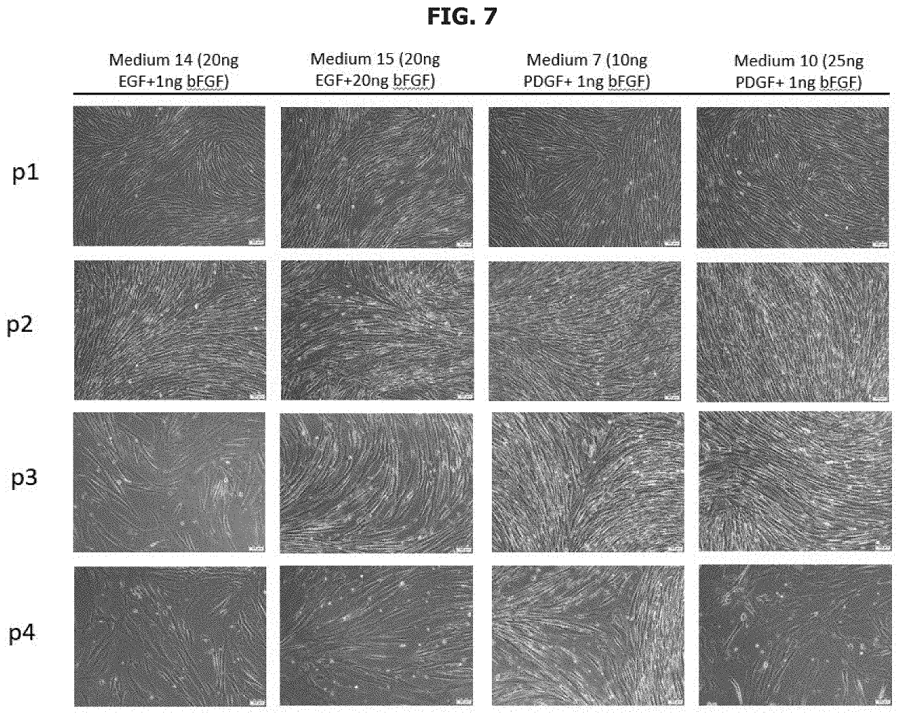

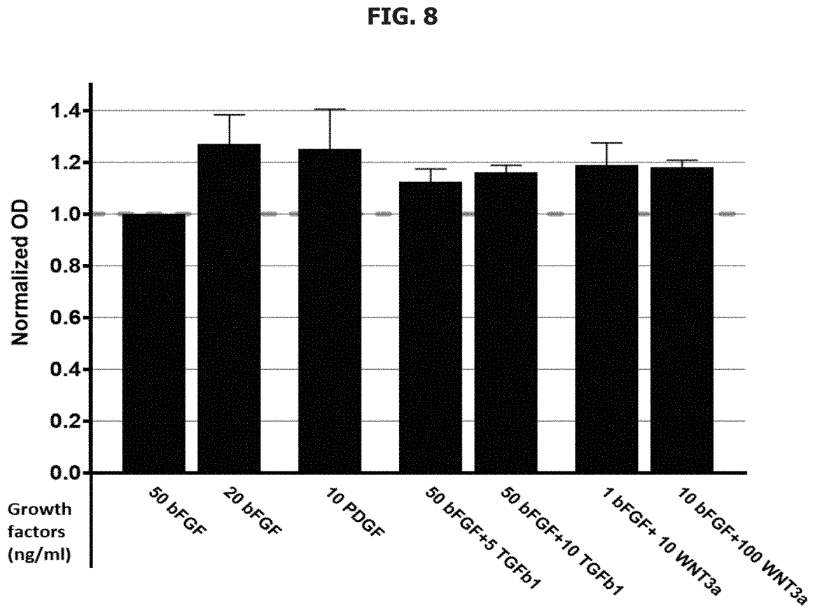

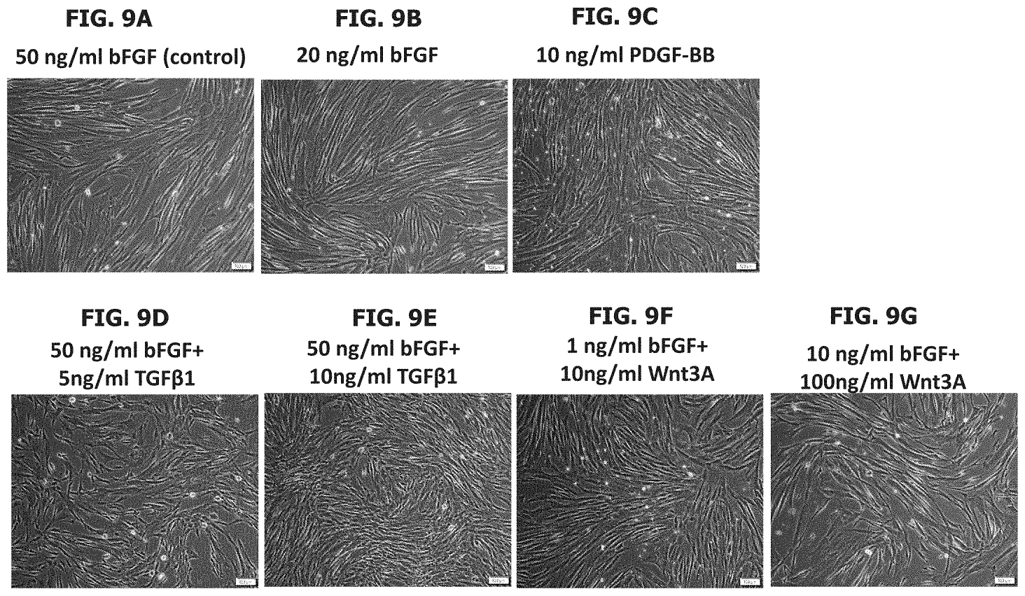

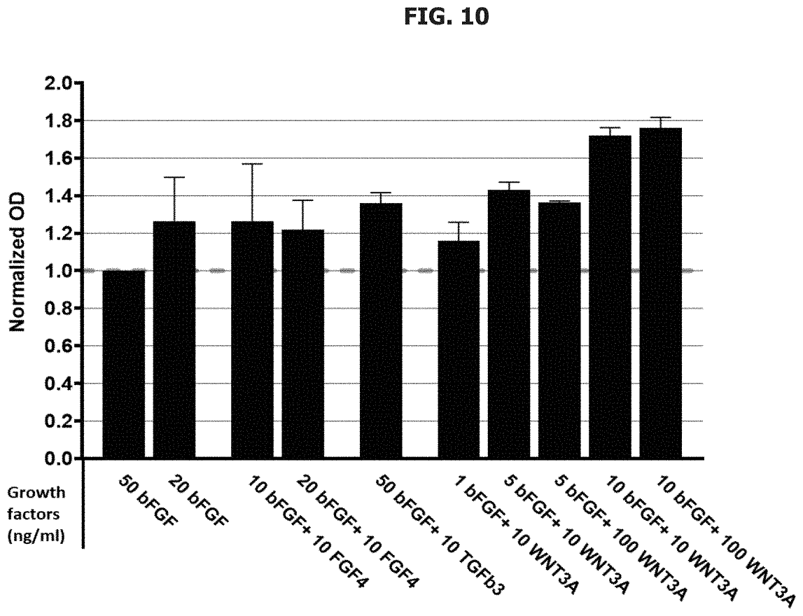

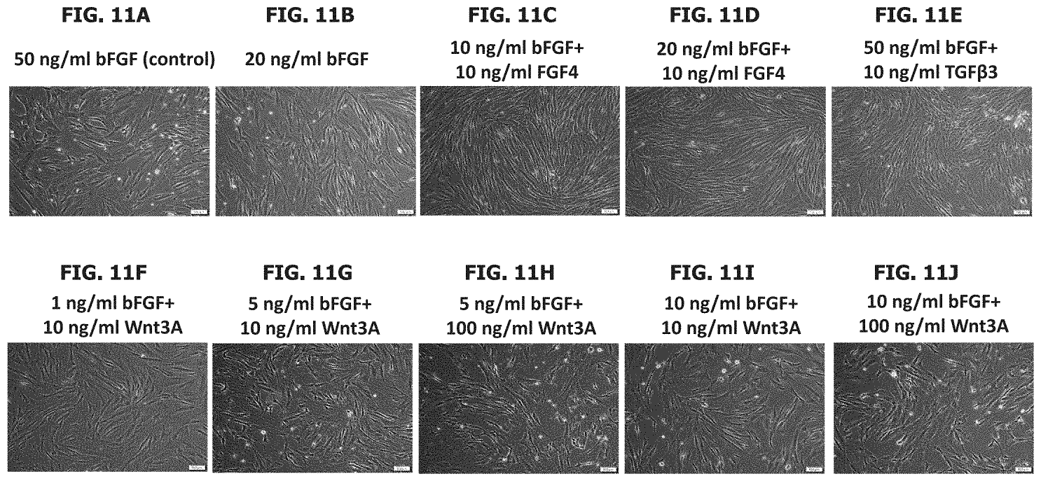

As illustrated in FIGS. 6A-C, the fold increase per day (i.e. the increase in cell number per day compared to the number of cells seeded at the start of each passage (P)), the population doubling time (PDT) (i.e. the doubling rate of the cells in 24 hours for each test) and the cumulative cell count (i.e. the addition of each cell count to the previous one to receive the total number of cells that would have been obtained had all the cells from the previous count had been seeded) illustrated the diverse effect of the growth factors combinations and concentrations. Representative morphology of the adult hMSC-UC cells cultured with MSC basal medium (MSC-Diff 0-F12) comprising different concentrations and combinations of bFGF, PDGF-BB and EGF are presented in FIG. 7. As illustrated in FIGS. 8 and 10, different concentrations and combinations of bFGF, TGFb1, TGFb3, PDGF-BB and WNT3a lead to an increase in cell proliferation in MSC-diff 0-F12 or MSC-Diff 0-HG, their representative morphology is presented in FIGS. 9A-G and 11A-J.

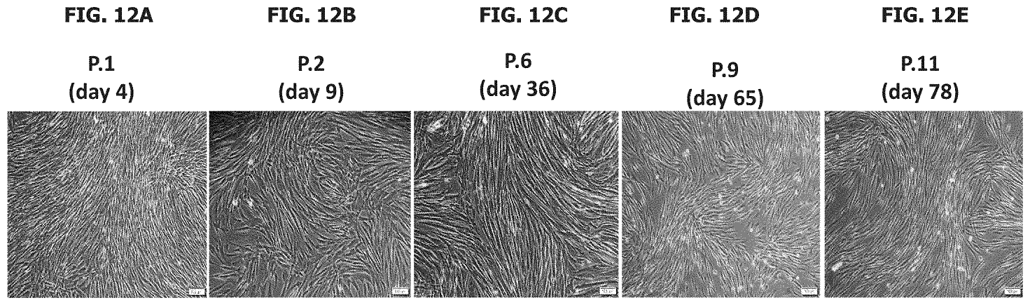

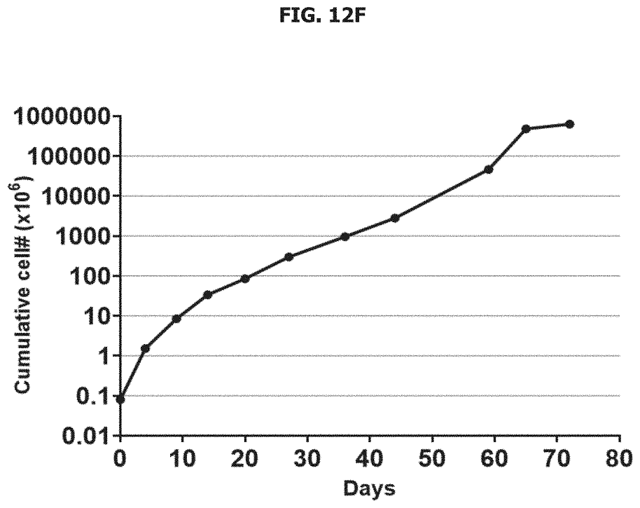

MSCs were further grown in MSC-Diff 51 media (see Table 5B, above) which resulted in maintained morphology following a long-term culture, as illustrated in passages 1 through 11 on days 4 to 78 of culture (see FIGS. 12A-E). Importantly, MSCs exhibited significant proliferation during a long term culture as illustrated for adult hMSC-UC cells over a period of 78 days (see FIG. 12F).

Furthermore, the growth and expansion of MSCs in MSC-Diff 52 media (see Table 5C, above) resulted in significant proliferation of MSCs concomitantly with maintained morphology following a long-term culture (data not shown).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. A defined culture medium comprising a basal medium and an effective amount of a knockout serum replacement (KoSR) and ITS (Insulin, Transferrin and Selenium), wherein the culture medium is serum-free, and further wherein the culture medium is capable of promoting differentiation of pluripotent stem cells (PSCs) into mesenchymal stem cells (MSCs) or supporting expansion of MSCs.

2. A culture medium comprising a basal medium comprising DMEM/F12 and MEM alpha at a volume ratio ranging between 0.4 to 2.3 DMEM/F12 to MEM alpha, wherein the culture medium is capable of promoting differentiation of pluripotent stem cells (PSCs) into mesenchymal stem cells (MSCs) or supporting expansion of MSCs.

3. The culture medium of claim 2, wherein said volume ratio of said DMEM/F12 to said MEM alpha ranges between 0.6 to 1.5 DMEM/F12 to MEM alpha.

4. The culture medium of claim 2, further comprising at least one of knockout serum replacement (KoSR) and ITS (Insulin, Transferrin and Selenium).

5. The culture medium of claim 1, wherein a concentration of said KoSR is 2.5-15%.

6. The culture medium of claim 1, wherein a concentration of said ITS is 0.1-3%.

7. The culture medium of claim 1, further comprising at least one of glucose, L-ascorbic acid and Sodium pyruvate.

8. The culture medium of claim 2, further comprising serum.

9. The culture medium of claim 8, wherein a concentration of said serum is 3-30%.

10. The culture medium of claim 1, further comprising basic fibroblast growth factor (bFGF).

11. The culture medium of claim 10, wherein said bFGF comprises FGF2 or FGF4.

12. The culture medium of claim 1, further comprising at least one of platelet-derived growth factor (PDGF), transforming growth factor beta (TGFβ), epidermal growth factor (EGF) and WNT-3a.

13. A culture medium comprising a basal medium comprising DMEM/F12 and MEM alpha at a volume ratio ranging between 0.6 to 1.5 DMEM/F12 to MEM alpha, a knockout serum replacement (KoSR) at a concentration of at least 3%, ITS (Insulin, Transferrin and Selenium) at a concentration of at least 0.1%, and serum at a concentration of at least 5%, and further wherein the culture medium is capable of promoting differentiation of pluripotent stem cells (PSCs) into mesenchymal stem cells (MSCs) or supporting expansion of MSCs.

14. A method of expanding mesenchymal stem cells (MSCs) without differentiation, the method comprising culturing the MSCs in the culture medium of claim 1, thereby culturing the MSCs under culturing conditions which allow expansion of the MSCs without differentiation.

15. The method of claim 14, wherein said culturing comprise a 2D culture.

16. The method of claim 14, wherein said culturing is for at least 5 passages.

17. The method of claim 14, wherein said MSCs are capable of differentiating into any one of an adipogenic lineage, an osteoblastic lineage, and a chondrogenic lineage.

18. A method of generating mesenchymal stem cells (MSCs) from pluripotent stem cells (PSCs), the method comprising culturing the PSCs in a suspension culture in the culture medium of claim 1 under culturing conditions suitable for differentiation of the PSCs to MSCs, thereby generating the MSCs.

19. A cell culture comprising MSCs and the culture medium of claim 1.

20. A cell culture comprising pluripotent stem cells (PSCs) and the culture medium of claim 1.
